(19) United States
(12) Patent Application Publication
(10) Pub. No.: US 2009/0281073 A1
(43) Pub. Date: Nov. 12, 2009

(54) HETEROAROMATIC DERIVATIVES USEFUL AS ANTICANCER AGENTS

(76) Inventors: Samit Kumar Bhattacharya, Groton, CT (US); Donna G. Wishka, Mystic, CT (US); Gonghua Pan, Old Lyme, CT (US)

Correspondence Address: PFIZER INC;Mary J Hosley, 150 EAST 42ND STREET, MS: 150/02/E112, NEW YORK, NY, 10017-5612, US

(21) Appl. No.: 11/722,325

(22) PCT Filed: Dec. 15, 2005
(86) PCT No.: PCT/IB2005/003933

§ 371 Date: Jun. 20, 2007

Related U.S. Application Data
(60) Provisional application No. 60/639,175, filed on Dec. 23, 2004.

(51) Int. Cl. A61K 31/519; C07D 471/04; A61P 35/00

(52) U.S. Cl. 514/210.16; 544/279; 544/230; 514/264.11

(57) ABSTRACT

This invention relates to compounds of Formula (I), and to pharmaceutically acceptable salts and solvates thereof, wherein Z, W, X, Y, V, R¹, R², and R³ are as defined herein. The invention also relates to methods of treating abnormal cell growth in mammals by administering the compounds of Formula (I) and to pharmaceutical compositions for treating such disorders which contain the compounds of Formula (I). The invention also relates to methods of preparing the compounds of Formula (I).

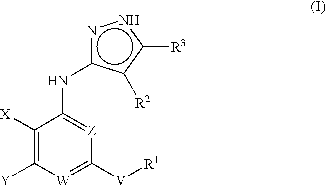

### BACKGROUND OF THE INVENTION

This invention relates to novel heteroaromatic derivatives that are useful in the treatment of abnormal cell growth, such as cancer, in mammals. This invention also relates to a method of using such compounds in the treatment of abnormal cell growth in mammals, especially humans, and to pharmaceutical compositions containing such compounds.

Cellular signal transduction is a fundamental cellular mechanism whereby external stimuli which regulate diverse cellular processes are relayed to the interior of cells. One of the key biochemical mechanisms of signal transduction in cells involves the reversible phosphorylation of proteins, which enables regulation of the activity of mature proteins by altering their structure and function.

The best characterized protein kinases in eukaryotes phosphorylate proteins on the alcohol moiety of serine, threonine and tyrosine residues. These kinases largely fall into two groups, those specific for phosphorylating serines and threonines (SIT kinases), and those specific for phosphorylating tyrosines. Some kinases, are referred to as “dual specificity” kinases, since they are able to phosphorylate on tyrosine as well as serine/threonine residues.

Protein kinases can also be characterized by their location within the cell. Some kinases are transmembrane receptor-type proteins capable of directly altering their catalytic activity in response to the external environment such as the binding of a ligand. Others are non-receptor-type proteins lacking any transmembrane domain. They can be found in a variety of cellular compartments from the inner surface of the cell membrane to the nucleus.

Many kinases are involved in regulatory cascades for cells wherein their substrates may include other kinases whose activities are regulated by their phosphorylation state. Ultimately the activity of some downstream effector is modulated by phosphorylation resulting from activation of such a pathway.

The serine/threonine (S/T) kinase family includes members found at all steps of various signaling cascades, including those involved in controlling cell growth, migration, differentiation and secretion of hormones, phosphorylation of transcription factors resulting in altered gene expression, muscle contraction, glucose metabolism, control of cellular protein synthesis, and regulation of the cell cycle.

One family of mitotic serine/threonine kinases is the Aurora (AUR) kinase family. The AUR kinase family has been found to be essential for providing signals that initiate and advance mitosis. It has been found that the Aurora kinases are overexpressed in tumor types, including colon cancer, breast cancer, and leukemia. Two primary isoforms of Aurora kinases have been identified and designated as form A and B. Aurora A is also known as Aurora-2 (AUR2), STK6, ARK1, Aurora/IPL1-related kinase, while Aurora B is also known as Aurora 1 or AUR1. The Aurora kinases have been characterized and identified in U.S. Pat. Nos. 5,962,312 and 5,972,676 (a divisional from the '312 patent) which relate to Aurora I (AUR-1) and Aurora 2 (AUR-2) polypeptides, nucleic acids encoding such polypeptides, cells, tissues and animals containing such nucleic acids, antibodies to such polypeptides, assays utilizing such polypeptides, and methods relating to all of the foregoing.

The overexpression of Aurora kinases, especially Aurora 2, in tumor cells provides an attractive target for drug intervention and the potential for a significant opportunity for controlling cell division in many types of cancer, and in particular for colon cancer and breast cancer. Applicants have now identified novel heteroaromatic Aurora kinase inhibitors which are able to modulate (reduce) that activity of the Aurora kinases in cancer cells.

### SUMMARY OF THE INVENTION

The present invention relates to compounds of Formula I:

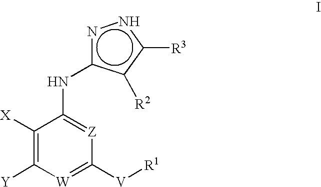

or a pharmaceutically acceptable salt, prodrug or solvate thereof, wherein:

W is N or CR⁴ and Z is N or CH, wherein at least one of W and Z is N;

R¹ is a 3 to 4 membered monocyclic ring selected from heterocyclyl or carbocyclyl, said heterocyclyl ring having 1 heteroatom selected from N, O, or S, wherein each substitutable carbon atom in the ring is independently substituted by oxo, -T-R⁴, or -L-Q-R⁴, and each substitutable nitrogen in the ring is independently substituted by R⁵; or R¹ is a 5 to 7 membered bicyclic ring selected from heteroaryl, heterocyclyl, or carbocyclyl, wherein said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵;

V is selected from the group consisting of a bond, —N(R⁵)—, —O—, —S—, —C(R⁶)₂—, and (C₁-C₁₀)alkyl, wherein a methylene unit of said (C₁-C₁₀)alkyl group is optionally replaced by a unit consisting of —O—, —S—, —N(R⁵)—, —CO—, —CONH—, —NHCO—, —SO₂, —SO₂NH—, —NHSO₂—, —CO₂—, —OC(O)—, —OC(O)NH—, and —NHCO₂;

X and Y are independently selected from -T-R⁴ or L-Q-R⁴, or X and Y are taken together with their intervening atoms to form a fused 5 to 7 membered ring having 0 to 3 ring heteroatoms selected from O, S or N, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵;

each T is independently selected from the group consisting of a bond and —(C₁-C₁₀)alkyl, wherein a methylene unit of said (C₁-C₁₀)alkyl group is optionally replaced by a unit consisting of —O—, —S—, —N(R⁵)—, —CO—, —CONH—, —NHCO—, —SO₂—, —SO₂NH—, —NHSO₂—, —CO₂—, —OC(O)—, —OC(O)NH—, and —NHCO₂—;

each Q is independently selected from —(C₁-C₁₀)alkyl;

each L is independently selected from the group consisting of —O—, —S—, —SO₂—, —N(R⁶)SO₂, —SO₂N(R⁶)—, —N(R⁸)—, —CO—, —CO—, —C(R⁶)OC(O)—, —C(R⁶)OC(O)N(R⁶)—, —N(R⁸)CO—, —N(R⁶)C(O)O—, —N(R⁶)CON(R⁶)—, —N(R⁸)SO₂N(R⁶)—, —N(R⁶)N(R⁶)—, —C(O)N(R⁸)—, —OC(O)N(R⁸)—, —C(R⁶)₂O—, —C(R⁶)₂S—, —C(R⁶)₂SO—, —C(R⁶)₂SO₂—, —C(R⁸)₂SO₂N(R⁶)—, —C(R⁶)₂N(R⁸)—, —C(R⁶)₂ N(R⁶)C(O)—, —C(R⁶)₂ N(R⁶)C(O)O—, —C(R⁶)═NN(R⁸)—, —C(R⁸)═N—O—, —C(R⁶)₂ N(R⁸)N(R⁶)—, —C(R⁶)₂ N(R⁸)SO₂N(R⁶)—, and —C(R⁸)₂N(R⁸)CON(R⁸)—;

R² and R³ are independently selected from -T-L-R⁶ and —R⁷; or R² and R³ are taken together with their intervening atoms to form a fused 5 to 9 membered ring having 0 to 3 ring heteroatoms selected from N, O, or S, wherein each substitutable ring carbon of said fused ring is independently substituted by halo, oxo, —CN, —NO₂, —R⁶, and -L-R⁶, and each substitutable ring nitrogen of said ring is independently substituted by R⁵;

R⁴ is selected from the group consisting of —H, halo, —CN, —R⁷, —OR⁷, —C(O)R⁷, —CO₂R⁷, —COCOR⁷, —NO₂, —S(O)R⁷, —SO₂R⁷, —SR⁷, —N(R⁵)₂, —CON(R⁵)₂, —SO₂N(R⁵)₂, —OC(O)R⁷, —N(R⁵)COR⁷, —N(R⁵)CO₂R⁷, —N(R⁵)C═SN(R⁵)₂, —N(R⁵)N(R⁵)₂, —C═NN(R⁶)₂, —C═NOR⁷, —N(R⁵)CON(R⁵)₂, —N(R⁵)SO₂N(R⁵)₂, —N(R⁷)SO₂R⁷, and —OC(O)N(R⁵)₂;

each R⁵ is independently selected from the group consisting of —R⁶, —COR⁸, —CO₂R⁶, —CON(R⁶)₂, and —SO₂R⁸;

each R⁶ is independently selected from H, —(C₁-C₁₀)alkyl, —(C₃-C₈)cycloalkyl, wherein said alkyl or cycloalkyl are independently optionally substituted by 1 to 3 substituents selected from R⁸; or two R⁶ groups on the same nitrogen atom are taken together with the nitrogen atom to form a 5 to 8 membered heterocyclyl or heteroaryl ring, wherein said heterocyclyl and heteroaryl rings have an additional 1 to 3 ring heteroatoms selected from N, O, or S; or two R⁶ groups on the same carbon atom are taken together with the carbon atom to form a 3 to 6 membered carbocyclic ring;

each R⁷ is independently selected from the group consisting of H, —(C₁-C₁₀)alkyl, —(C₂-C₆)alkenyl, —(C₂-C₈)alkynyl, —(CH₂)ₙ(C₃-C₈)cycloalkyl, —(CH₂)ₙ(C₆-C₁₀)aryl, —(CH₂)ₙ(5 to 10 membered heteroaryl), and —(CH₂)ₙ(5 to 10 membered heterocyclyl), wherein said heteroaryl and heterocyclyl rings having 1 to 3 ring heteroatoms selected from N, O, or S, wherein said alkyl, alkenyl, alkynyl, cycloalkyl, aryl, heteroaryl, and heterocyclyl are independently optionally substituted by 1 to 3 substituents selected from R⁸;

n is an integer from 0 to 6;

each R⁸ is selected from the group consisting of halo, —CN, —OR⁹, —SR⁹, —SO₂R⁹, —N(R⁹)SO₂R⁹, —SO₂N(R⁹)₂, —N(R⁹)₂, —COR⁹, —CO₂R⁹, —C(R⁹)OC(O)R⁹, —C(R⁹)OC(O)N(R⁹)₂, —N(R⁹)COR⁹, —N(R⁹)C(O)OR⁹, —N(R⁹)CON(R⁹)₂, —N(R⁹)SO₂N(R⁹)₂, —N(R⁹)N(R⁹)₂, —C(O)N(R⁹)₂, —OC(O)N(R⁹)₂, —C(R⁹)₂OR⁹, —C(R⁹)₂SR⁹, —C(R⁹)₂SOR⁹, —C(R⁹)₂SO₂R⁹, —C(R⁹)₂SO₂N(R⁹)₂, —C(R⁹)₂N(R⁹)₂, —C(R⁹)₂N(R⁹)C(O)R⁹, —C(R⁹)₂ N(R⁹)C(O)OR⁹, —C(R⁹)═NN(R⁹)₂, —C(R⁹)═NOR⁹, —C(R⁹)₂ N(R⁹)N(R⁹)₂, —C(R⁹)₂N(R⁹)SO₂N(R⁹)₂, and —C(R⁹)₂N(R⁹)CON(R⁹)₂; and

each R⁹ is independently selected from H, —(C₁-C₁₀)alkyl, —(C₃-C₈)cycloalkyl or two R⁹ groups on the same nitrogen atom may be taken together with the nitrogen atom to form a 5 to 8 membered heterocyclyl or heteroaryl ring, wherein said heterocyclyl and heteroaryl rings having 1 to 3 ring heteroatoms selected from N, O, or S, or two R⁹ groups on the same carbon atom may be taken together with the carbon atom to form a 3 to 6 membered carbocyclic ring.

In one preferred embodiment of the present invention W is N and Z is CH.

In another preferred embodiment of the present invention W is N and Z is N.

In one preferred embodiment of the present invention W is CR⁴ and Z is N.

In one embodiment of the present invention V is selected from the group consisting of a bond, —N(R⁵)—, —O—, —C(R⁶)₂—, and (C₁-C₁₀)alkyl, wherein a methylene unit of said (C₁-C₁₀)alkyl group is optionally replaced by a unit consisting of —O—, —S—, —N(R⁵)—, —CO—, —CONH—, —NHCO—, —SO₂—, —SO₂NH—, —NHSO₂—, —CO₂—, —OC(O)—, —OC(O)NH—, and —NHCO₂.

In another preferred embodiment of the present invention V is selected from the group consisting of a bond, —N(R⁵)—, —O—, and (C₁-C₁₀)alkyl, wherein a methylene unit of said (C₁-C₁₀)alkyl group is optionally replaced by a unit consisting of —O—, —S—, —N(R⁵)—, —CO—, —CONH—, —NHCO—, —SO₂—, —SO₂NH—, —NHSO₂—, —CO₂—, —OC(O)—, —OC(O)NH—, and —NHCO₂.

In another preferred embodiment of the present invention V is selected from the group consisting of a bond, —N(R⁵)— and (C₁-C₁₀)alkyl, wherein a methylene unit of said (C₁-C₁₀)alkyl group is optionally replaced by a unit consisting of —O—, —S—, —N(R⁵)—, —CO—, —CONH—, —NHCO—, —SO₂—, —SO₂NH—, —NHSO₂—, —CO₂—, —OC(O)—, —OC(O)NH—, and —NHCO₂.

In another preferred embodiment of the present invention V is selected from the group consisting of a bond and (C₁-C₁₀)alkyl, wherein a methylene unit of said (C₁-C₁₀)alkyl group is optionally replaced by a unit consisting of —O—, —S—, —N(R⁵)—, —CO—, —CONH—, —NHCO—, —SO₂—, —SO₂NH—, —NHSO₂—, —CO₂—, —OC(O)—, —OC(O)NH—, and —NHCO₂.

In another preferred embodiment of the present invention V is (C₁-C₁₀)alkyl, wherein a methylene unit of said (C₁-C₁₀)alkyl group is optionally replaced by a unit consisting of —O—, —S—, —N(R⁵)—, —CO—, —CONH—, —NHCO—, —SO₂—, —SO₂NH—, —NHSO₂—, —CO₂—, —OC(O)—, —OC(O)NH—, and —NHCO₂.

In a more preferred embodiment of the present invention V is a bond.

In a preferred embodiment of the present invention R¹ is a 3 membered monocyclic ring selected from heterocyclyl or carbocyclyl, said heterocyclyl ring having 1 to 4 heteroatoms selected from N, O, or S, wherein each substitutable carbon atom in the ring is independently substituted by oxo, -T-R⁴, or -L-Q-R⁴, and each substitutable nitrogen in the ring is independently substituted by R⁵; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 4 membered monocyclic ring selected from heterocyclyl or carbocyclyl, said heterocyclyl ring having 1 to 4 heteroatoms selected from N, O, or S, wherein each substitutable carbon atom in the ring is independently substituted by oxo, -T-R⁴, or -L-Q-R⁴, and each substitutable nitrogen in the ring is independently substituted by R⁵; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 3 membered carbocyclyl ring, wherein each substitutable carbon atom in the ring is independently substituted by oxo, -T-R⁴, or -L-Q-R⁴; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R.

In another preferred embodiment of the present invention R¹ is a 4 membered carbocyclyl ring, wherein each substitutable carbon atom in the ring is independently substituted by oxo, -T-R⁴, or -L-Q-R⁴; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁶; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 3 membered heterocyclyl ring, said heterocyclyl ring having 1 to 2 heteroatoms selected from N or S, wherein each substitutable carbon atom in the ring is independently substituted by oxo, -T-R⁴, or -L-Q-R⁴, and each substitutable nitrogen in the ring is independently substituted by R⁵; or R¹ is a to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁶.

In another preferred embodiment of the present invention R¹ is a 4 membered heterocyclyl ring, said heterocyclyl ring having 1 to 2 heteroatoms selected from N or S, wherein each substitutable carbon atom in the ring is independently substituted by oxo, -T-R⁴, or L-Q-R⁴, and each substitutable nitrogen in the ring is independently substituted by R⁵; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or A is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 3 membered heterocyclyl ring, said heterocyclyl ring having 1 heteroatom selected from N or S, wherein each substitutable carbon atom in the ring is independently substituted by oxo, -T-R⁴, or -L-Q-R⁴, and each substitutable nitrogen in the ring is independently substituted by R⁵; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 4 membered heterocyclyl ring, said heterocyclyl ring having 1 heteroatom selected from N or S, wherein each substitutable carbon atom in the ring is independently substituted by oxo, -T-R⁴, or -L-Q-R⁴, and each substitutable nitrogen in the ring is independently substituted by R⁵; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or A is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 3 membered heterocyclyl ring, said heterocyclyl ring having 1 N heteroatom, wherein each substitutable carbon atom in the ring is independently substituted by oxo, -T-R⁴, or -L-Q-R⁴, and each substitutable nitrogen in the ring is independently substituted by R⁵; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 4 membered heterocyclyl ring, said heterocyclyl ring having 1 N heteroatom, wherein each substitutable carbon atom in the ring is independently substituted by oxo, -T-R⁴, or -L-Q-R⁴, and each substitutable nitrogen in the ring is independently substituted by R⁵; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 3 membered heterocyclyl ring, said heterocyclyl ring having 1 S heteroatom, wherein each substitutable carbon atom in the ring is independently substituted by oxo, -T-R⁴, or -L-Q-R⁴; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 4 membered heterocyclyl ring, said heterocyclyl ring having 1 S heteroatom, wherein each substitutable carbon atom in the ring is independently substituted by oxo, -T-R⁴, or -L-Q-R⁴; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R.

In another preferred embodiment of the present invention R¹ is a 3 membered heterocyclyl ring, said heterocyclyl ring having 1 O heteroatom, wherein each substitutable carbon atom in the ring is independently substituted by oxo, -T-R⁴, or -L-Q-R⁴; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 4 membered heterocyclyl ring, said heterocyclyl ring having 1 O heteroatom, wherein each substitutable carbon atom in the ring is independently substituted by oxo, -T-R⁴, or -L-Q-R⁴; or R¹ is a 5 to 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 7 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered bicyclic ring selected from heteroaryl, heterocyclyl, or carbocyclyl, said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered heteroaryl ring, said heteroaryl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered heterocyclyl ring, said heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention wherein R¹ is a 5 to 6 membered carbocyclyl ring, wherein each substitutable ring carbon in the ring is independently substituted by 1 to 2 substituents selected from oxo, -T-R⁴, or -L-Q-R⁴.

In another preferred embodiment of the present invention R¹ is a 5 to 6 membered bicyclic ring selected from heteroaryl, heterocyclyl, or carbocyclyl, said heteroaryl or heterocyclyl ring having 1 to 2 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁶.

In another preferred embodiment of the present invention R¹ is a 5 to 6 membered heteroaryl ring, said heteroaryl ring having 1 to 2 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered heterocyclyl ring, said heterocyclyl ring having 1 to 2 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered bicyclic ring selected from heteroaryl, heterocyclyl, or carbocyclyl, said heteroaryl or heterocyclyl ring having 1 heteroatom selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered heteroaryl ring, said heteroaryl ring having 1 heteroatom selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered heterocyclyl ring, said heterocyclyl ring having 1 heteroatom selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered bicyclic ring selected from heteroaryl, heterocyclyl, or carbocyclyl, said heteroaryl or heterocyclyl ring having 1 N heteroatom, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered heteroaryl ring, said heteroaryl ring having 1 N heteroatom, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered heterocyclyl ring, said heterocyclyl ring having 1 N heteroatom, wherein each substitutable ring carbon in the ring is independently substituted by 1 to 2 substituents selected from oxo, -T-R⁴, or -L-Q-R⁴, and each substitutable ring nitrogen in the ring is independently substituted by R⁶.

In another preferred embodiment of the present invention R¹ is a 5 to 6 membered bicyclic ring selected from heteroaryl, heterocyclyl, or carbocyclyl, said heteroaryl or heterocyclyl ring having 1 S heteroatom, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered heteroaryl ring, said heteroaryl ring having 1 S heteroatom, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered heterocyclyl ring, said heterocyclyl ring having 1 S heteroatom, wherein each substitutable ring carbon in the ring is independently substituted by 1 to 2 substituents selected from oxo, -T-R⁴, or -L-Q-R⁴, and each substitutable ring nitrogen in the ring is independently substituted by R⁶.

In another preferred embodiment of the present invention R¹ is a 5 to 6 membered bicyclic ring selected from heteroaryl, heterocyclyl, or carbocyclyl, said heteroaryl or heterocyclyl ring having 1 O heteroatom, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 5 to 6 membered heteroaryl ring, said heteroaryl ring having 10 heteroatom, wherein each substitutable ring carbon in the ring is independently substituted by 1 to 2 substituents selected from oxo, -T-R⁴, or -L-Q-R⁴, and each substitutable ring nitrogen in the ring is independently substituted by R.

In another preferred embodiment of the present invention R¹ is a 5 to 6 membered heterocyclyl ring, said heterocyclyl ring having 1 O heteroatom, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 to 10 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 to 8 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 8 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is 7 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is 6 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 to 10 membered spiroheterocyclyl ring, said spiroheterocyclyl ring having 1 to 2 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R.

In another preferred embodiment of the present invention R¹ is a 6 to 8 membered spiroheterocyclyl ring, said spiroheterocyclyl ring having 1 to 2 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 8 membered spiroheterocyclyl ring, said spiroheterocyclyl ring having 1 to 2 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is 7 membered spiroheterocyclyl ring, said spiroheterocyclyl ring having 1 to 2 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁶.

In another preferred embodiment of the present invention R¹ is 6 membered spiroheterocyclyl ring, said spiroheterocyclyl ring having 1 to 2 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 to 10 membered spiroheterocyclyl ring, said spiroheterocyclyl ring having 1 to 2 N heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 to 8 membered spiroheterocyclyl ring, said spiroheterocyclyl ring having 1 to 2 N heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 8 membered spiroheterocyclyl ring, said spiroheterocyclyl ring having 1 to 2 N heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 7 membered spiroheterocyclyl ring, said spiroheterocyclyl ring having 1 to 2 N heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 membered spiroheterocyclyl ring, said spiroheterocyclyl ring having 1 to 2 N heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 to 10 membered spiroheterocyclyl ring, said spiroheterocyclyl ring having 1 to 2 S heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 to 8 membered spiroheterocyclyl ring, said spiroheterocyclyl ring having 1 to 2 S heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 8 membered spiroheterocyclyl ring, said spiroheterocyclyl ring having 1 to 2 S heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 7 membered spiroheterocyclyl ring, said spiroheterocyclyl ring having 1 to 2 S heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 membered spiroheterocyclyl ring, said spiroheterocyclyl ring having 1 to 2 S heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 to 10 membered spiroheterocyclyl ring, said spiroheterocyclyl ring having 1 to 2 O heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 to 8 membered spiroheterocyclyl ring, said spiroheterocyclyl ring having 1 to 2 O heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 8 membered spiroheterocyclyl ring, said spiroheterocyclyl ring having 1 to 2 O heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 7 membered spiroheterocyclyl ring, said spiroheterocycyl ring having 1 to 2 O heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In another preferred embodiment of the present invention R¹ is a 6 membered spiroheterocyclyl ring, said spiroheterocyclyl ring having 1 to 2 O heteroatoms, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵.

In a more preferred embodiment of the present invention R¹ is selected from the group consisting of:

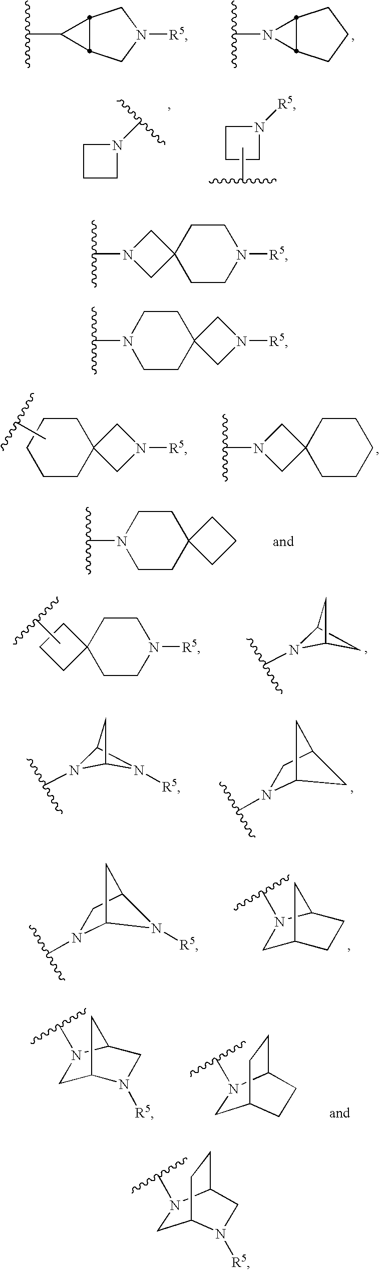

wherein each substitutable ring carbon in the ring is independently substituted by 1 to 2 substituents selected from oxo, -T-R⁴, or -L-Q-R⁴.

In a most preferred embodiment of the present invention R¹ is selected from the group consisting of:

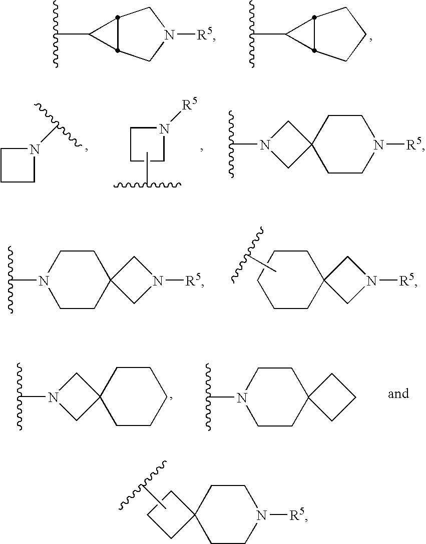

wherein each substitutable ring carbon in the ring is independently substituted by 1 to 2 substituents selected from oxo, -T-R⁴, or -L-Q-R⁴.

In another most preferred embodiment of the present invention R¹ is selected from the group consisting of:

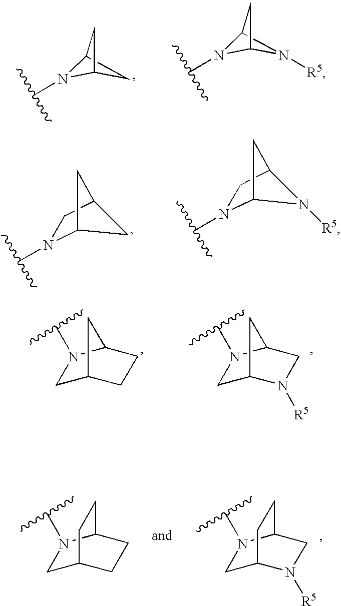

wherein each substitutable ring carbon in the ring is independently substituted by 1 to 2 substituents selected from oxo, -T-R⁴, or -L-Q-R⁴.

In a preferred embodiment of the present invention X and Y are independently selected from -L-Q-R⁴; or X and Y are taken together with their intervening atoms to form a fused 5 to 7 membered ring having 0 to 3 ring heteroatoms selected from O, S or N, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having 0 to 3 ring heteroatoms selected from O, S or N, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁶.

In another preferred embodiment of the present invention X and Y are taken together with their intervening atoms to form a fused 5 to 6 membered ring having 0 to 3 ring heteroatoms selected from O, S or N, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 7 membered ring having 0 to 3 ring heteroatoms selected from O, S or N, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 6 membered ring having 0 to 3 ring heteroatoms selected from O, S or N, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 membered ring having 0 to 3 ring heteroatoms selected from O, S or N, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁶.

In another preferred embodiment of the present invention X and Y are taken together with their intervening atoms to form a fused 5 to 7 membered ring having 0 to 2 ring heteroatoms selected from O, S or N, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having 0 to 1 ring heteroatoms selected from O, S or N, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having 1 ring heteroatom selected from O, S or N, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having O ring heteroatom, wherein each substitutable ring carbon of said fused ring is independently substituted by 1 to 2 substituents selected from oxo, -T-R⁴, or -L-Q-R⁴.

In another preferred embodiment of the present invention X and Y are taken together with their intervening atoms to form a fused 5 to 7 membered ring having 0 to 3 ring N atoms, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having 0 to 2 ring N atoms, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having 1 ring N atom, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having 0 to 3 ring S atoms, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having 0 to 2 ring S atoms, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having 1 ring S atom, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having 0 to 3 ring O atoms, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In another preferred embodiment of the present invention X and Y are taken together with their intervening atoms to form a fused 5 to 7 membered ring having 0 to 2 ring O atoms, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁶.

In another preferred embodiment of the present invention X and Y are taken together with their intervening atoms to form a fused 5 to 7 membered ring having 1 ring O atom, wherein each substitutable ring carbon of said fused ring is independently substituted by 1 to 2 substituents selected from oxo, -T-R⁴, or -L-Q-R⁴, and each substitutable ring nitrogen of said fused ring is independently substituted by R⁵.

In a more preferred embodiment of the present invention X and Y are taken together with their intervening atoms to form a fused ring selected from:

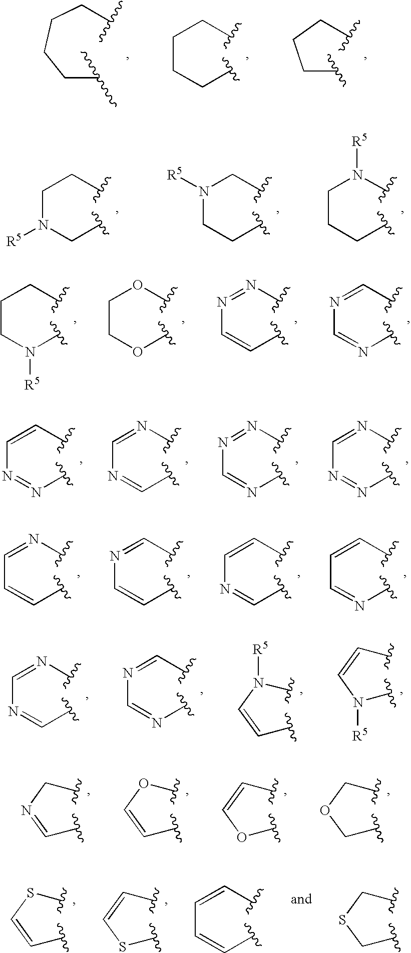

wherein each substitutable ring carbon of said fused ring is independently substituted by 1 to 2 substituents selected from oxo, -T-R⁴, or -L-Q-R⁴.

In a more preferred embodiment of the present invention X and Y are taken together with their intervening atoms to form a fused ring selected from:

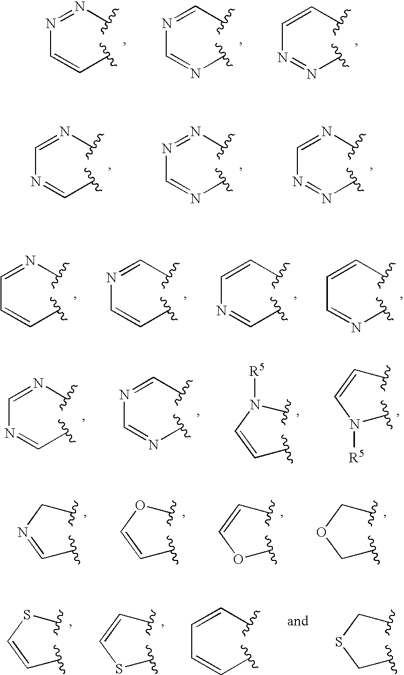

wherein each substitutable ring carbon of said fused ring is Independently substituted by 1 to 2 substituents selected from oxo, -T-R⁴, or -L-Q-R⁴.

In a most preferred embodiment of the present invention X and Y are taken together with their intervening atoms to form a fused ring selected from:

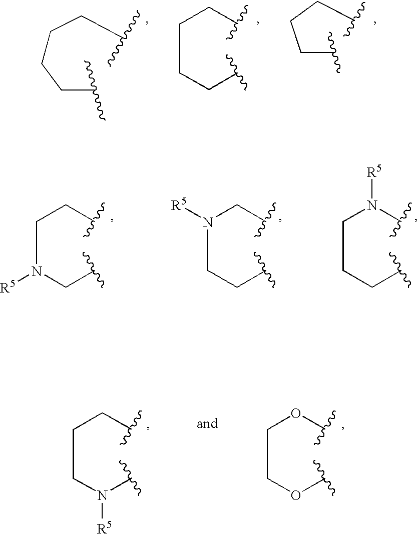

wherein each substitutable ring carbon of said fused ring is independently substituted by 1 to 2 substituents selected from oxo, -T-R⁴, or -L-Q-R⁴.

In a most preferred embodiment of the present invention X and Y are taken together with their intervening atoms to form a fused ring selected from:

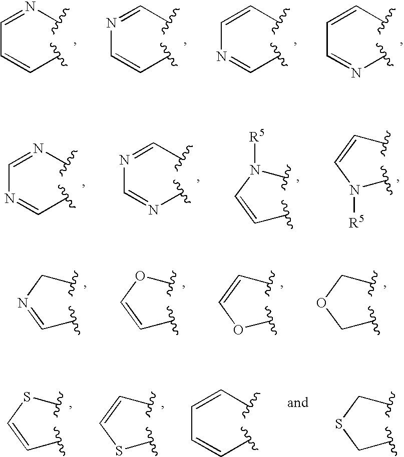

wherein each substitutable ring carbon of said fused ring is independently substituted by 1 to 2 substituents selected from oxo, -T-R⁴, or -L-Q-R⁴.

In another most preferred embodiment of the present invention X and Y are taken together with their intervening atoms to form a fused ring selected from:

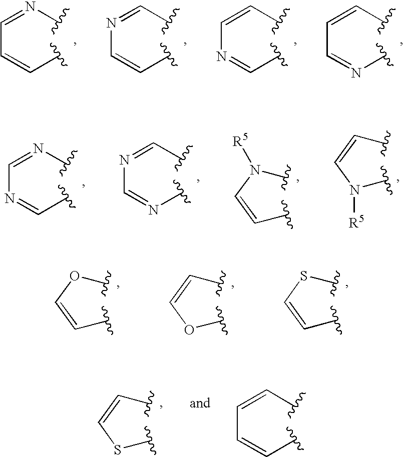

wherein each substitutable ring carbon of said fused ring is independently substituted by 1 to 2 substituents selected from oxo, -T-R⁴, or -L-Q-R⁴.

In another most preferred embodiment of the present invention X and Y are taken together with their intervening atoms to form a fused ring selected from:

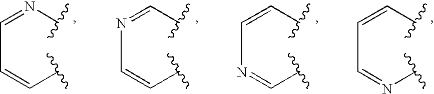

wherein each substitutable ring carbon of said fused ring is independently substituted by 1 to 2 substituents selected from oxo, -T-R⁴, or -L-Q-R⁴.

In another most preferred embodiment of the present invention X and Y are taken together with their intervening atoms to form a fused ring selected from:

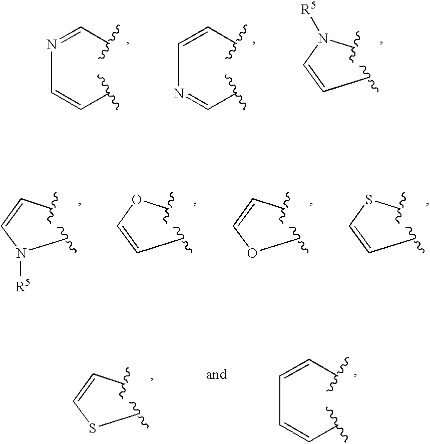

wherein each substitutable ring carbon of said fused ring is independently substituted by 1 to 2 substituents selected from oxo, -T-R⁴, or -L-Q-R⁴.

In another most preferred embodiment of the present invention X and Y are taken together with their intervening atoms to form a fused ring selected from:

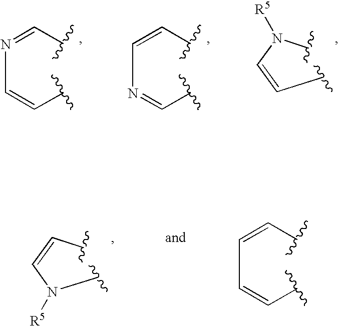

wherein each substitutable ring carbon of said fused ring is independently substituted by 1 to 2 substituents selected from oxo, -T-R⁴, or -L-Q-R⁴.

In another most preferred embodiment of the present invention X and Y are taken together with their intervening atoms to form a fused ring selected from:

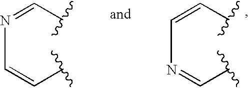

wherein each substitutable ring carbon of said fused ring is independently substituted by 1 to 2 substituents selected from oxo, -T-R⁴, or -L-Q-R⁴.

In a most preferred embodiment of the present invention X and Y are taken together with their intervening atoms to form a fused ring selected from:

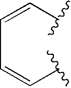

wherein each substitutable ring carbon of said fused ring is independently substituted by 1 to 2 substituents selected from oxo, -T-R⁴, or -L-Q-R⁴.

In another most preferred embodiment of the present invention X and Y are taken together with their intervening atoms to form a fused ring selected from:

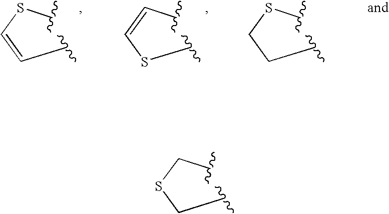

wherein each substitutable ring carbon of said fused ring is independently substituted by 1 to 2 substituents selected from oxo, -T-R⁴, or -L-Q-R⁴.

In another most preferred embodiment of the present invention X and Y are taken together with their intervening atoms to form a fused ring selected from:

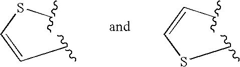

wherein each substitutable ring carbon of said fused ring is independently substituted by 1 to 2 substituents selected from oxo, -T-R⁴, or -L-Q-R⁴.

In another most preferred embodiment of the present invention X and Y are taken together with their intervening atoms to form a fused ring selected from:

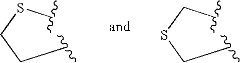

wherein each substitutable ring carbon of said fused ring is independently substituted by 1 to 2 substituents selected from oxo, -T-R⁴, or -L-Q-R⁴.

In another most preferred embodiment of the present invention X and Y are independently selected from -T-R⁴ or L-Q-R⁴.

In another most preferred embodiment of the present invention X and Y are independently selected from -T-R⁴.

In another most preferred embodiment of the present invention X and Y are independently selected from -T-R⁴.

In another most preferred embodiment of the present invention T is —(C₁-C₁₀)alkyl, wherein a methylene unit of said (C₁-C₁₀)alkyl group is optionally replaced by a group consisting of —O—, —S—, —N(R⁵)—, —CO—, —CONH—, —NHCO—, —SO₂—, —SO₂NH—, —NHSO₂—, —CO₂—, —OC(O)—, —OC(O)NH—, and —NHCO₂—.

In a most preferred embodiment of the present invention T is a bond.

In a preferred embodiment of the present invention R⁴ is selected from the group consisting of H, halo, —CN, —R⁷, —OR⁷, —C(O)R⁷, —CO₂R⁷, —COCOR⁷, —NO₂, —S(O)R⁷, —SO₂R⁷, —SR⁷, —N(R⁵)₂, —CON(R⁵)₂, —SO₂N(R⁶)₂, —OC(O)R⁷, —N(R⁵)COR⁷, —N(R⁵)CO₂R⁷, —N(R⁵)C═SN(R⁵)₂, —N(R⁵)N(R⁵)₂, —N(R⁵)CON(R⁵)₂, —N(R⁵)SO₂N(R⁵)₂, —N(R⁷)SO₂R⁷, and —OC(O)N(R⁵)₂.

In a most preferred embodiment of the present invention R⁴ is selected from the group consisting of —H, halo, and —CN.

In another preferred embodiment of the present invention R⁴ is selected from the group consisting of —R⁷, —OR⁷, —C(O)R¹, —CO₂R⁷, —COCOR⁷, —NO₂, —S(O)R⁷, —SO₂R⁷, —SR⁷, —N(R⁵)₂, —CON(R⁵)₂, —SO₂N(R⁵)₂, —OC(O)R⁷, —N(R⁵)COR⁷, —N(R⁵)CO₂R⁷, —N(R⁵)C═SN(R⁵)₂, —N(R⁵)N(R⁵)₂, —N(R⁵)CON(R⁵)₂, —N(R⁵)SO₂N(R⁵)₂, —N(R⁷)SO₂R⁷, and —OC(O)N(R⁵)₂.

In another preferred embodiment of the present invention R² and R³ are independently selected from -T-L-R⁶, and —R⁷.

In another preferred embodiment of the present invention R² and R³ are taken together with their intervening atoms to form a fused 5 to 9 membered ring having 0 to 3 ring heteroatoms selected from N, O, or S, wherein each substitutable ring carbon of said fused ring is independently substituted by halo, oxo, —CN, —NO₂, —R⁶, and -L-R⁸, and each substitutable ring nitrogen of said ring is independently substituted by R⁵.

In another preferred embodiment of the present invention R² and R³ are taken together with their intervening atoms to form a fused 5 to 7 membered ring having 0 to 3 ring heteroatoms selected from N, O, or S, wherein each substitutable ring carbon of said fused ring is independently substituted by halo, oxo, —CN, —NO₂, —R⁸, and -L-R⁸, and each substitutable ring nitrogen of said ring is independently substituted by R⁵.

In another preferred embodiment of the present invention R² and R³ are taken together with their intervening atoms to form a fused 5 to 6 membered ring having 0 to 3 ring heteroatoms selected from N, O, or S, wherein each substitutable ring carbon of said fused ring is independently substituted by halo, oxo, —CN, —NO₂, —R⁶, and -L-R⁶, and each substitutable ring nitrogen of said ring is independently substituted by R⁵.

Specific embodiments of the compounds of Formula I include those selected from the group consisting of:
Cyclopropanesulfonic acid {5-methyl-3-[4-(5-methyl-1H-pyrazol-3-ylamino)-pyrido[2,3-d]pyrimidin-2-yl]-3-aza-bicyclo[3.1.0]hex-1-yl}-amide;
1-{2-[4-(5-Methyl-2H-pyrazol-3-ylamino)-pyrido[2,3-d]pyrimidin-2-yl]-2,7-diaza-spiro[3.5]non-7-yl}-ethanone;
2-((1S,4S)-5-benzyl-2,5-diaza-bicyclo[2.2.1]heptan-2-yl)-N-(3-cyclopropyl-1H-pyrazol-5-yl)thieno[3,2-d]pyrimidin-4-amine;
exo-(S)—N2-(7-Aza-bicyclo[2.2.1]hept-2-yl)-N4-(5-methyl-1H-pyrazol-3-yl)-thieno[3,2-d]pyrimidine-2,4-diamine hydrochloride;
exo-(R)—N2-7-Aza-bicyclo[2.2.1]hept-2-yl)-N4-(5-methyl-2H-pyrazol-3-yl)-thieno[3,2-d]pyrimidine-2,4-diamine hydrochloride;
exo-benzyl-7-(4-(3-methyl-1H-pyrazol-5-ylamino)thieno[3,2-d]pyrimidin-2-yl)-7-aza-bicyclo[2.2.1]heptan-2(S)-ylcarbamate;
{(1S,4S)-5-[4-(5-Cyclopropyl-2H-pyrazol-3-ylamino)-thieno[3,2-d]pyrimidin-2-yl]-2,5-diaza-bicyclo[2.2.1]hept-2-yl}-pyridin-3-yl-methanone;
{(1S,4S)-5-[4-(5-Cyclopropyl-2H-pyrazol-3-ylamino)-thieno[3,2-d]pyrimidin-2-yl]-2,5-diaza-bicyclo[2.2.1]hept-2-yl}-pyrazin-2-yl-methanone;
1-{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-methoxy-ethanone;
1-{(1R,5S,6S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-methyl-propan-1-one;
1-{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-phenyl-ethanone;
(5-Cyclopropyl-2H-pyrazol-3-yl)-{2-[(1S,4S)-5-(propane-2-sulfonyl)-2,5-diaza-bicyclo[2.2.1]hept-2-yl]-thieno[3,2-d]pyrimidin-4-yl}-amine;
[2-((1S,4S)-5-Cyclopropanesulfonyl-2,5-diaza-bicyclo[2.2.1]hept-2-yl)-thieno[3,2-d]pyrimidin-4-yl]-(5-cyclopropyl-2H-pyrazol-3-yl)-amine;
(1S,4S)-5-[4-(5-Cyclopropyl-2H-pyrazol-3-ylamino)-thieno[3,2-d]pyrimidin-2-yl]-2,5-diaza-bicyclo[2.2.1]heptane-2-carboxylic acid phenylamide;
(1R,5S)-6-[4-(5-Cyclopropyl-2H-pyrazol-3-ylamino)-thieno[3,2-d]pyrimidin-2-ylamino]-3-aza-bicyclo[3.1.0]hexane-3-carboxylic acid benzylamide;
(5-Cyclopropyl-2H-pyrazol-3-yl)-[2-((1S,4S)-5-propyl-2,5-diaza-bicyclo[2.2.1]hept-2-yl)-thieno[3,2-d]pyrimidin-4-yl]-amine;
(5-Cyclopropyl-2H-pyrazol-3-yl)-{2-[(1S,4S)-5-(1-methyl-1H-imidazol-2-ylmethyl)-2,5-diaza-bicyclo[2.2.1]hept-2-yl]-thieno[3,2-d]pyrimidin-4-yl}-amine;
(5-Cyclopropyl-2H-pyrazol-3-yl)-{2-[(1S,4S)-5-(1H-imidazol-2-ylmethyl)-2,5-diaza-bicyclo[2.2.1]hept-2-yl]-thieno[3,2-d]pyrimidin-4-yl}-amine;
[2-((1S,4S)-5-Benzyl-2,5-diaza-bicyclo[2.2.1]hept-2-yl)-thieno[3,2-d]pyrimidin-4-yl]-(5-cyclopropyl-2H-pyrazol-3-yl)-amine;
(5-Cyclopropyl-2H-pyrazol-3-yl)-[2-((1S,4S)-5-phenethyl-2,5-diaza-bicyclo[2.2.1]hept-2-yl)-thieno[3,2-d]pyrimidin-4-yl]-amine;
(5-Cyclopropyl-2H-pyrazol-3-yl)-{2-[(1S,4S)-5-(tetrahydro-furan-2-ylmethyl)-2,5-diaza-bicyclo[2.2.1]hept-2-yl]-thieno[3,2-d]pyrimidin-4-yl}-amine;
(5-Cyclopropyl-2H-pyrazol-3-yl)-[2-((1S,4S)-5-isoxazol-3-ylmethyl-2,5-diaza-bicyclo[2.2.1]hept-2-yl)-thieno[3,2-d]pyrimidin-4-yl]-amine;
{(1S,4S)-5-[4-(5-Cyclopropyl-2H-pyrazol-3-ylamino)-thieno[3,2-d]pyrimidin-2-yl]-2,5-diaza-bicyclo[2.2.1]hept-2-yl}-acetic acid ethyl ester;
{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-acetic acid ethyl ester;
N4-(5-Methyl-1H-pyrazol-3-yl)-N2-[(1R,5S,6S)-3-(propane-2-sulfonyl)-3-aza-bicyclo[3.1.0]hex-6-yl]-pyrimidine-2,4-diamine;

N4-(5-Methyl-1H-pyrazol-3-yl)-N2-[(1S,5R)-3-(propane-1-sulfonyl)-3-aza-bicyclo[3.1.0]hex-6-yl]-pyrimidine-2,4-diamine;
3-{(1R,5S)-6-[4-(5-Methyl-1H-pyrazol-3-ylamino)-pyrimidin-2-ylamino]-3-aza-bicyclo[3.1.0]hexane-3-sulfonyl}-benzonitrile;
3-Cyano-N-{(1R,5S)-3-[4-(5-methyl-1H-pyrazol-3-ylamino)-pyrimidin-2-yl]-3-aza-bicyclo[3.1.0]hex-6-yl}-benzenesulfonamide;
N2-[(1R,5S)-3-(3,5-Dimethyl-1H-pyrazole-4-sulfonyl)-3-aza-bicyclo[3.1.0]hex-6-yl]-N4-(5-methyl-1H-pyrazol-3-yl)-pyrimidine-2,4-diamine;
(1R,5S)-6-[4-(5-Methyl-1H-pyrazol-3-ylamino)-pyrimidin-2-ylamino]-3-aza-bicyclo[3.1.0]hexane-3-carboxylic acid (1H-indol-3-yl)-amide;
(1R,5S)-6-[4-(5-Methyl-1H-pyrazol-3-ylamino)-pyrimidin-2-ylamino]-3-aza-bicyclo[3.1.0]hexane-3-carboxylic acid ((R)-1-phenyl-ethyl)-amide;
(1R,2S,4S)-2-[4-(5-Methyl-1H-pyrazol-3-ylamino)-thieno[3,2-d]pyrimidin-2-ylamino]-7-aza-bicyclo[2.2.1]heptane-7-carboxylic acid tert-butyl ester,
N2-(1R,2S)-7-Aza-bicyclo[2.2.1]hept-2-yl-N4-(5-methyl-1H-pyrazol-3-yl)-thieno[3,2-d]pyrimidine-2,4-diamine;
(1S,2R,4R)-2-[4-(5-Methyl-2H-pyrazol-3-ylamino)-thieno[3,2-d]pyrimidin-2-ylamino]-7-aza-bicyclo[2.2.1]heptane-7-carboxylic acid tert-butyl ester;
N2-(1S,2R,4R)-7-Aza-bicyclo[2.2.1]hept-2-yl-N4-(5-methyl-2H-pyrazol-3-yl)-thieno[3,2-d]pyrimidine-2,4-diamine;
{(1R,2S,4S)-7-[4-(5-Methyl-2H-pyrazol-3-ylamino)-thieno[3,2-d]pyrimidin-2-yl]-7-aza-bicyclo[2.2.1]hept-2-yl}-carbamic acid benzyl ester;
(2-Azetidin-1-yl-pyrido[2,3-d]pyrimidin-4-yl)-(5-methyl-2H-pyrazol-3-yl)-amine;
{(1R,5S)-3-[4-(5-Methyl-2H-pyrazol-3-ylamino)-pyrido[2,3-d]pyrimidin-2-yl]-3-aza-bicyclo[3.1.0]hex-6-yl}-carbamic acid tert-butyl ester;
[2-(2-Aza-bicyclo[2.2.1]hept-2-yl)-pyrido[2,3-d]pyrimidin-4-yl]-(5-methyl-2H-pyrazol-3-yl)-amine;
(5-Methyl-2H-pyrazol-3-yl)-[2-(2-propyl-aziridin-1-yl)-pyrido[2,3-d]pyrimidin-4-yl]-amine;
4-{1-[4-(5-Methyl-2H-pyrazol-3-ylamino)-pyrido[2,3-d]pyrimidin-2-yl]-azetidin-3-yl}-piperazine-1-carboxylic acid tert-butyl ester;
2-[4-(5-Met-2H-pyrazol-3-ylamino)-pyrido[2,3-d]pyrimidin-2-yl]-2,7-diaza-spiro[3,5]nonane-7-carboxylic acid tert-butyl ester,
2-Methoxy-N-{(1S,5R)-3-[4-(5-methyl-2H-pyrazol-3-ylamino)-pyrido[2,3-d]pyrimidin-2-yl]-3-aza-bicyclo[3.1.0]hex-6-yl}-acetamide;
5-{2-[(1S,5R)-6-(2-Methoxy-acetylamino)-3-aza-bicyclo[3.1.0]hex-3-yl]-pyrido[2,3-d]pyrimidin-4-ylamino}-3-methyl-pyrazole-1-carboxylic acid ethyl ester,
N-((1S,5R)-3-[4-(5-Methyl-2H-pyrazol-3-ylamino)-pyrido[2,3-d]pyrimidin-2-yl]-3-aza-bicyclo[3.1.0]hex-6-yl)-acetamide;
5-[2-((1S,5R)-6-Acetylamino-3-aza-bicyclo[3.1.0]hex-3-yl)-pyrido[2,3-d]pyrimidin-4-ylamino]-3-methyl-pyrazole-1-carboxylic acid ethyl ester;
Cyclopropanecarboxylic acid {(1S,5R)-3-[4-(5-methyl-2H-pyrazol-3-ylamino)-pyrido[2,3-d]pyrimidin-2-yl]-3-aza-bicyclo[3.1.0]hex-6-yl}-amide;
5-[2-((1S,5R)-6-Methanesulfonylamino-3-aza-bicyclo[3.1.0]hex-3-yl)-pyrido[2,3-d]pyrimidin-4-ylamino]-3-methyl-pyrazole-1-carboxylic acid ethyl ester;
Isopropyl-3-((1S,5R)-3-[4-(5-methyl-2H-pyrazol-3-ylamino)-pyrido[2,3-d]pyrimidin-2-yl]-3-aza-bicyclo[3.1.0]hex-6-yl)-urea;
5-{2-[(1S,5R)-6-(3-Isopropyl-ureido)-3-aza-bicyclo[3.1.0]hex-3-yl]-pyrido[2,3-d]pyrimidin-4-ylamino}-3-methyl-pyrazole-1-carboxylic acid ethyl ester,
5-{2-[(1S,5R)-6-(3-Ethyl-thioureido)-3-aza-bicyclo[3.1.0]hex-3-yl]-pyrido[2,3-d]pyrimidin-4-ylamino}-3-methyl-pyrazole-1-carboxylic acid ethyl ester,
2-Methoxy-1-{2-[4-(5-methyl-2H-pyrazol-3-ylamino)-pyrido[2,3-d]pyrimidin-2-yl]-2,7-diaza-spiro[3.5]non-7-yl}-ethanone;
[2-(7-Methanesulfonyl-2,7-diaza-spiro[3.5]non-2-yl)-pyrido[2,3-d]pyrimidin-4-yl]-(5-methyl-2H-pyrazol-3-yl)-amine;
[2-((1R,4R)-5-Methanesulfonyl-2,5-diaza-bicyclo[2.2.1]hept-2-yl)-pyrido[2,3-d]pyrimidin-4-yl]-(5-methyl-2H-pyrazol-3-yl)-amine;
(1R,5S)-3-[4-(5-Methyl-1H-pyrazol-3-ylamino)-pyrido[2,3-d]pyrimidin-2-yl]-6-morpholin-4-yl-3-aza-bicyclo[3.1.0]hexane-6-carbonitrile;
[(1R,4S)-2-(2-Aza-bicyclo[2.2.1]hept-2-yl)-pyrido[2,3-d]pyrimidin-4-yl]-(5-methyl-1H-pyrazol-3-yl)-amine;
{(1S,5R)-3-[4-(5-Methyl-1H-pyrazol-3-ylamino)-pyrido[2,3-d]pyrimidin-2-yl]-3-aza-bicyclo[3.1.0]hex-1-yl}-carbamic acid tert-butyl ester;
[2-((1S,5R)-1-Amino-3-aza-bicyclo[3.1.0]hex-3-yl)pyrido[2,3-d]pyrimidin-4-yl]-(5-methyl-1H-pyrazol-3-yl)-amine;
1-{(1R,4S)-5-[4-(5-Methyl-2H-pyrazol-3-ylamino)-pyrido[2,3-d]pyrimidin-2-yl]-2,5-diaza-bicyclo[2.2.1]hept-2-yl}-ethanone;
2-Methoxy-1-{(1R,4S)-5-[4-(5-methyl-2H-pyrazol-3-ylamino)-pyrido[2,3-d]pyrimidin-2-yl]-2,5-diaza-bicyclo[2.2.1]hept-2-yl}-ethanone;
2-Methoxy-N-{1-[4-(5-methyl-2H-pyrazol-3-ylamino)-pyrido[2,3-d]pyrimidin-2-yl]-azetidin-3-ylmethyl}-acetamide;
Cyclopropanesulfonic acid {1-[4-(5-methyl-2H-pyrazol-3-ylamino)-pyrido[2,3-d]pyrimidin-2-yl]-azetidin-3-ylmethyl}-amide;
Cyclopropanesulfonic acid {(1S,5R)-3-[4-(5-methyl-1H-pyrazol-3-ylamino)-pyrido[2,3-d]pyrimidin-2-yl]-3-aza-bicyclo[3.1.0]hex-1-yl}-amide;
[2-((1R,5S)-6-Benzylamino-3-aza-bicyclo[3.1.0]hex-3-yl)-pyrido[2,3-d]pyrimidin-4-yl]-(5-methyl-1H-pyrazol-3-yl)-amine;
N-{(1S,5R)-3-[6,7-Dimethoxy-4-(5-methyl-2H-pyrazol-3-ylamino)-quinazolin-2-yl]-3-aza-bicyclo[3.1.0]hex-6-yl}-methanesulfonamide;
1-{2-[6,7-Dimethoxy-4-(5-methyl-2H-pyrazol-3-ylamino)-quinazolin-2-yl]-2,7-diaza-spiro[3,5]non-7-yl}-2-methoxy-ethanone;
1-{2-[6,7-Dimethoxy-4-(5-methyl-2H-pyrazol-3-ylamino)-quinazolin-2-yl]-2,7-diaza-spiro[3.5]non-7-yl}-ethanone;
Cyclopropyl-{2-[6,7-dimethoxy-4-(5-methyl-2H-pyrazol-3-ylamino)-quinazolin-2-yl]-2,7-diaza-spiro[3.5]non-7-yl}-methanone;
1-{2-[6,7-Dimethoxy-4-(5-methyl-2H-pyrazol-3-ylamino)-quinazolin-2-yl]-2,7-diaza-spiro[3.5]non-7-yl}-2-methyl-propan-1-one;
[2-(7-Methanesulfonyl-2,7-diaza-spiro[3.5]non-2-yl)-6,7-dimethoxy-quinazolin-4-yl]-(5-methyl-2H-pyrazol-3-yl)-amine;
2-[6,7-Dimethoxy-4-(5-methyl-2H-pyrazol-3-ylamino)-quinazolin-2-yl]-2,7-diaza-spiro[3.5]nonane-7-carboxylic acid methyl ester;
[2-(7-Ethanesulfonyl-2,7-diaza-spiro[3.5]non-2-yl)-6,7-dimethoxy-quinazolin-4-yl]-(5-methyl-2H-pyrazol-3-yl)-amine;
2-[6,7-Dimethoxy-4-(5-methyl-2H-pyrazol-3-ylamino)-quinazolin-2-yl]-2,7-diaza-spiro[3,5]nonane-7-carboxylic acid ethylamide;
N-{1-[6,7-Dimethoxy-4-(5-methyl-2H-pyrazol-3-ylamino)-quinazolin-2-yl]-azetidin-3-yl}-2-methoxy-acetamide;
N-{1-[6,7-Dimethoxy-4-(5-methyl-2H-pyrazol-3-ylamino)-quinazolin-2-yl]-azetidin-3-yl}-methanesulfonamide;
Ethanesulfonic acid {1-[6,7-dimethoxy-4-(5-methyl-2H-pyrazol-3-ylamino)-quinazolin-2-yl]-azetidin-3-yl}-amide;
2-Methoxy-1-{(1S,4S)-5-[8-methoxy-4-(5-methyl-2H-pyrazol-3-ylamino)-quinazolin-2-yl]-2,5-diaza-bicyclo[2.2.1]hept-2-yl}-ethanone;
[2-((1S,4S)-5-Methanesulfonyl-2,5-diaza-bicyclo[2.2.1]hept-2-yl)-8-methoxy-quinazolin-4-yl]-(5-methyl-2H-pyrazol-3-yl)-amine;
(1S,4S)-5-[8-Methoxy-4-(5-methyl-2H-pyrazol-3-ylamino)-quinazolin-2-yl]-2,5-diaza-bicyclo[2.2.1]heptane-2-carboxylic acid methyl ester;
{(1R,5S)-3-[8-Methoxy-4-(5-methyl-1H-pyrazol-3-ylamino)-quinazolin-2-yl]-3-aza-bicyclo[3.1.0]hex-6-yl}-carbamic acid tert-butyl ester,
{(1R,5S)-3-[8-Methoxy-4-(5-methyl-1H-pyrazol-3-ylamino)-quinazolin-2-yl]-3-aza-bicyclo[3.1.0]hex-1-yl}-carbamic acid tert-butyl ester;

and the pharmaceutically acceptable salts and solvates of the foregoing compounds.

Specific preferred embodiments of the compounds of Formula I include those selected from the group consisting of:
2-((1S,4S)-5-benzyl-2,5-diaza-bicyclo[2.2.1]heptan-2-yl)-N-(3-cyclopropyl-1H-pyrazol-5-yl)thieno[3,2-d]pyrimidin-4-amine;
exo-(S)—N2-(7-Aza-bicyclo[2.2.1]hept-2-yl)-N4-(5-methyl-1H-pyrazol-3-yl)-thieno[3,2-d]pyrimidine-2,4-diamine hydrochloride;
exo-(R)—N2-7-Aza-bicyclo[2.2.1]hept-2-yl)-N4-(5-methyl-2H-pyrazol-3-yl)-thieno[3,2-d]pyrimidine-2,4-diamine hydrochloride;
exo-benzyl-7-(4-(3-methyl-1H-pyrazol-5-ylamino)thieno[3,2-d]pyrimidin-2-yl)-7-aza-bicyclo[2.2.1]heptan-2(S)-ylcarbamate;
{(1S,4S)-5-[4-(5-Cyclopropyl-2H-pyrazol-3-ylamino)-thieno[3,2-d]pyrimdin-2-yl]-2,5-diaza-bicyclo[2.2.1]hept-2-yl}-pyridin-3-yl-methanone;
{(1S,4S)-5-[4-(5-Cyclopropyl-2H-pyrazol-3-ylamino)-thieno[3,2-d]pyrimidin-2-yl]-2,5-diaza-bicyclo[2.2.1]hept-2-yl}-pyrazin-2-yl-methanone;
1-{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-methoxy-ethanone;
1-{(1R,5S,6S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-methyl-propan-1-one;
1-{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-phenyl-ethanone;
(5-Cyclopropyl-2H-pyrazol-3-yl)-{2-[(1S,4S)-5-(propane-2-sulfonyl)-2,5-diaza-bicyclo[2.2.1]hept-2-yl]-thieno[3,2-d]pyrimidin-4-yl}-amine;
[2-((1S,4S)-5-Cyclopropanesulfonyl-2,5-diaza-bicyclo[2.2.1]hept-2-yl)-thieno[3,2-d]pyrimidin-4-yl]-(5-cyclopropyl-2H-pyrazol-3-yl)-amine;
(1S,4S)-5-[4-(5-Cyclopropyl-2H-pyrazol-3-ylamino)-thieno-[3,2-d]pyrimidin-2-yl]-2,5-diaza-bicyclo[2.2.1]heptane-2-carboxylic acid phenylamide;
(1R,5S)-6-[4-(5-Cyclopropyl-2H-pyrazol-3-ylamino)-thieno[3,2-d]pyrimidin-2-ylamino]-3-aza-bicyclo[3.1.0]hexane-3-carboxylic acid benzylamide;
(5-Cyclopropyl-2H-pyrazol-3-yl)-[2-((1S,4S)-5-propyl-2,5-diaza-bicyclo[2.2.1]hept-2-yl)-thieno[3,2-d]pyrimidin-4-yl]-amine;
(5-Cyclopropyl-2H-pyrazol-3-yl)-{2-[(1S,4S)-5-(1-methyl-1H-imidazol-2-ylmethyl)-2,5-diaza-bicyclo[2.2.1]hept-2-yl]-thieno[3,2-d]pyrimidin-4-yl}-amine;

(5-Cyclopropyl-2H-pyrazol-3-yl)-{2-[(1S,4S)-5-(1H-imidazol-2-ylmethyl)-2,5-diaza-bicyclo[2.2.1]hept-2-yl]-thieno[3,2-d]pyrimidin-4-yl}-amine;
[2-((1S,4S)-5-Benzyl-2,5-diaza-bicyclo[2.2.1]hept-2-yl)-thieno[3,2-d]pyrimidin-4-yl]-(5-cyclopropyl-2H-pyrazol-3-yl)-amine;
(5-Cyclopropyl-2H-pyrazol-3-yl)-[2-((1S,4S)-5-phenethyl-2,5-diaza-bicyclo[2.2.1]hept-2-yl)-thieno[3,2-d]pyrimidin-4-yl]-amine;
(5-Cyclopropyl-2H-pyrazol-3-yl)-{2-[(1S,4S)(tetrahydro-furan-2-ylmethyl)-2,5-diaza-bicyclo[2.2.1]hept-2-yl]-thieno[3,2-d]pyrimidin-4-yl}-amine;
(5-Cyclopropyl-2H-pyrazol-3-yl)-[2-((1S,4S)-5-isoxazol-3-ylmethyl-2,5-diaza-bicyclo[2.2.1]hept-2-yl)thieno[32-d]pyrimidin-4-yl]-amine;
{(1S,4S)-5-[4-(5-Cyclopropyl-2H-pyrazol-3-ylamino)-thieno[3,2-d]pyrimidin-2-yl]-2,5-diaza-bicyclo[2.2.1]hept-2-yl}-acetic acid ethyl ester;
{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-acetic acid ethyl ester;
(1R,2S,4S)-2-[4-(5-Methyl-1H-pyrazol-3-ylamino)-thieno[3,2-d]pyrimidin-2-ylamino]-7-aza-bicyclo[2.2.1]heptane-7-carboxylic acid tert-butyl ester;
N2-(1R,2S)-7-Aza-bicyclo[2.2.1]hept-2-yl-N4-(5-methyl-1H-pyrazol-3-yl)-thieno[3,2-d]pyrimidine-2,4-diamine;
(1S,2R,4R)-2-[4-(5-Methyl-2H-pyrazol-3-ylamino)-thieno[3,2-d]pyrimdin-2-ylamino]-7-aza-bicyclo[2.2.1]heptane-7-carboxylic acid tert-butyl ester,
N2-(1S,2R,4R)-7-Aza-bicyclo[2.2.1]hept-2-yl-N4-(5-methyl-2H-pyrazol-3-yl)-thieno[3,2-d]pyrimidine-2,4-diamine;
{(1R,2S,4S)-7-[4-(5-Methyl-2H-pyrazol-3-ylamino)-thieno[3,2-d]pyrimdin-2-yl]-7-aza-bicyclo[2.2.1]hept-2-yl}-carbamic acid benzyl ester, and the pharmaceutically acceptable salts and solvates of the foregoing compounds.

Specific preferred embodiments of the compounds of Formula I include those selected from the group consisting of:
Cyclopropanesulfonic acid {5-methyl-3-[4-(5-methyl-1H-pyrazol-3-ylamino)-pyrido[2,3-d]pyrimidin-2-yl]-3-aza-bicyclo[3.1.0]hex-1-yl}-amide;
1-{2-[4-(5-Me-2H-pyrazol-3-ylamino)-pyrido[2,3-d]pyrimidin-2-yl]-2,7-diaza-spiro[3.5]non-7-yl}-ethanone;
(2-Azetidin-1-yl-pyrido[2,3-d]pyrimidin-4-yl)-(5-methyl-2H-pyrazol-3-yl)-amine;
{(1R,5S)-3-[4-(5-Methyl-2H-pyrazol-3-ylamino)-pyrido[2,3-d]pyrimidin-2-yl]-3-aza-bicyclo[3.1.0]hex-6-yl}-carbamic acid tert-butyl ester;
[2-(2-Aza-bicyclo[2.2.1]hept-2-yl)-pyrido[2,3-d]pyrimidin-4-yl]-(5-methyl-2H-pyrazol-3-yl)-amine;
(5-Methyl-2H-pyrazol-3-yl)-[2-(2-propyl-aziridin-1-yl)-pyrido[2,3-d]pyrimidin-4-yl]-amine;
4-{1-[4-(5-Methyl-2H-pyrazol-3-ylamino)-pyrido[2,3-d]pyrimidin-2-yl]-azetidin-3-yl}-piperazine-1-carboxylic acid tert-butyl ester;
2-[4-(5-Methyl-2H-pyrazol-3-ylamino)-pyrido[2,3-d]pyrimidin-2-yl]-2,7-diaza-spiro[3.5]nonane-7-carboxylic acid tert-butyl ester
2-Methoxy-N-{(1S,5R)-3-[4-(5-methyl-2H-pyrazol-3-ylamino)-pyrido[2,3-d]pyrimdin-2-yl]-3-aza-bicyclo[3.1.0]hex-6-yl}-acetamide;
5-{2-[(1S,5R)-6-(2-Methoxy-acetylamino)-3-aza-bicyclo[3.1.0]hex-3-yl]-pyrido[2,3-d]pyrimidin-4-ylamino}-3-methyl-pyrazole-1-carboxylic acid ethyl ester
N-{(1S,5R)-3-[4-(5-Methyl-2H-pyrazol-3-ylamino)-pyrido[2,3-d]pyrimidin-2-yl]-3-aza-bicyclo[3.1.0]hex-6-yl}-acetamide;
5-[2-((1S,5R)-6-Acetylamino-3-aza-bicyclo[3.1.0]hex-3-yl)-pyrido[2,3-d]pyrimidin-4-ylamino]-3-methyl-pyrazole-1-carboxylic acid ethyl ester;
Cyclopropanecarboxylic acid {(1S,5R)-3-[4-(5-methyl-2H-pyrazol-3-ylamino)-pyrido[2,3-d]pyrimidin-2-yl]-3-aza-bicyclo[3.1.0]hex-6-yl}-amide;
5-[2-((1S,5R)-6-Methanesulfonylamino-3-aza-bicyclo[3.1.0]hex-3-yl)-pyrido[2,3-d]pyrimidin-4-ylamino]-3-methyl-pyrazole-1-carboxylic acid ethyl ester;
Isopropyl-3-{(1S,5R)-3-[4-(5-methyl-2H-pyrazol-3-ylamino)-pyrido[2,3-d]pyrimdin-2-yl]-3-aza-bicyclo[3.1.0]hex-6-yl}-urea;
5-{2-[(1S,5R)-6-(3-Isopropyl-ureido)-3-aza-bicyclo[3.1.0]hex-3-yl]-pyrido[2,3-d]pyrimidin-4-ylamino}-3-methyl-pyrazole-1-carboxylic acid ethyl ester,
5-{2-[(1S,5R)-6-(3-Ethyl-thioureido)-3-aza-bicyclo[3.1.0]hex-3-yl]-pyrido[2,3-d]pyrimidin-4-ylamino}-3-methyl-pyrazole-1-carboxylic acid ethyl ester;
2-Methoxy-1-{2-[4-(5-methyl-2H-pyrazol-3-ylamino)-pyrido[2,3-d]pyrimidin-2-yl]-2,7-diaza-spiro[3.5]non-7-yl}-ethanone;
[2-(7-Methanesulfonyl-2,7-diaza-spiro[3.5]non-2-yl)-pyrido[2,3-d]pyrimidin-4-yl]-(5-methyl-2H-pyrazol-3-yl)-amine;
[2-((1R,4R)-5-Methanesulfonyl-2,5-diaza-bicyclo[2.2.1]hept-2-yl)-pyrido[2,3-d]pyrimidin-4-yl]-(5-methyl-2H-pyrazol-3-yl)-amine;
(1R,5S)-3-[4-(5-Methyl-1H-pyrazol-3-ylamino)-pyrido[2,3-d]pyrimidin-2-yl]-6-morpholin-4-yl-3-aza-bicyclo[3.1.0]hexane-6-carbonitrile;
[(1R,4S)-2-(2-Aza-bicyclo[2.2.1]hept-2-yl)-pyrido[2,3-d]pyrimidin-4-yl]-(5-methyl-1H-pyrazol-3-yl)-amine;
{(1S,5R)-3-[4-(5-Methyl-1H-pyrazol-3-ylamino)-pyrido[2,3-d]pyrimidin-2-yl]-3-aza-bicyclo[3.1.0]hex-1-yl}-carbamic acid tert-butyl ester;
[2-((1S,5R)-1-Amino-3-aza-bicyclo[3.1.0]hex-3-yl)-pyrido[2,3-d]pyrimidin-4-yl]-(5-methyl-1H-pyrazol-3-yl)-amine;
1-{(1R,4S)-5-[4-(5-Methyl-2H-pyrazol-3-ylamino)-pyrido[2,3-d]pyrimidin-2-yl]-2,5-diaza-bicyclo[2.2.1]hept-2-yl}-ethanone;
2-Methoxy-1-{(1R,4S)-5-[4-(5-methyl-2H-pyrazol-3-ylamino)-pyrido[2,3-d]pyrimidin-2-yl]-2,5-diaza-bicyclo[2.2.1]hept-2-yl}-ethanone;
2-Methoxy-N-{1-[4-(5-methyl-2H-pyrazol-3-ylamino)-pyrido[2,3-d]pyrimidin-2-yl]-azetidin-3-ylmethyl}-acetamide;
Cyclopropanesulfonic acid {1-[4-(5-methyl-2H-pyrazol-3-ylamino)-pyrido[2,3-d]pyrimidin-2-yl]-azetidin-3-ylmethyl}-amide;
Cyclopropanesulfonic acid {(1S,5R)-3-[4-(5-methyl-1H-pyrazol-3-ylamino)-pyrido[2,3-d]pyrimidin-2-yl]-3-aza-bicyclo[3.1.0]hex-1-yl}-amide;
[2-((1R,5S)-6-Benzylamino-3-aza-bicyclo[3.1.0]hex-3-yl)-pyrido[2,3-d]pyrimidin-4-yl]-(5-methyl-1H-pyrazol-3-yl)-amine; and the pharmaceutically acceptable salts and solvates of the foregoing compounds.

Specific preferred embodiments of the compounds of Formula I include those selected from the group consisting of:
N-{(1S,5R)-3-[6,7-Dimethoxy-4-(5-methyl-2H-pyrazol-3-ylamino)-quinazolin-2-yl]-3-aza-bicyclo[3.1.0]hex-6-yl}-methanesulfonamide;
1-{2-[6,7-Dimethoxy-4-(5-methyl-2H-pyrazol-3-ylamino)-quinazolin-2-yl]-2,7-diaza-spiro[3,5]non-7-yl}-2-methoxy-ethanone;
1-{2-[6,7-Dimethoxy-5-methyl-2H-pyrazol-3-ylamino)-quinazolin-2-yl]-2,7-diaza-spiro[3.5]non-7-yl}-ethanone;
Cyclopropyl-{2-[6,7-dimethoxy-4-(5-methyl-2H-pyrazol-3-ylamino)-quinazolin-2-yl]-2,7-diaza-spiro[3.5]non-7-yl}-methanone;
1-{2-[6,7-Dimethoxy-4-(5-methyl-2H-pyrazol-3-ylamino)-quinazolin-2-yl]-2,7-diaza-spiro[3.5]non-7-yl}-2-methyl-propan-1-one;
[2-(7-Methanesulfonyl-2,7-diaza-spiro[3.5]non-2-yl)-6,7-dimethoxy-quinazolin-4-yl]-(5-methyl-2H-pyrazol-3-yl)-amine;
2-[6,7-Dimethoxy-4-(5-methyl-2H-pyrazol-3-ylamino)-quinazolin-2-yl]-2,7-diaza-spiro[3.5]nonane-7-carboxylic acid methyl ester;
[2-(7-Ethanesulfonyl-2,7-diaza-spiro[3.5]non-2-yl)-6,7-dimethoxy-quinazolin-4-yl]-(5-methyl-2H-pyrazol-3-yl)-amine;
2-[6,7-Dimethoxy-4-(5-methyl-2H-pyrazol-3-ylamino)-quinazolin-2-yl]-2,7-diaza-spiro[3.5]nonane-7-carboxylic acid ethylamide;
N-{1-[6,7-Dimethoxy-4-(5-methyl-2H-pyrazol-3-ylamino)-quinazolin-2-yl]-azetidin-3-yl}-2-methoxy-acetamide;
N-{1-(6,7-Dimethoxy-4-(5-methyl-2H-pyrazol-3-ylamino)-quinazolin-2-yl-azetidin-3-yl}-methanesulfonamide;
Ethanesulfonic acid {1-[6,7-dimethoxy-4-(5-methyl-2H-pyrazol-3-ylamino)-quinazolin-2-yl]-azetidin-3-yl}-amide;
2-Methoxy-1-{(1S,4S)-5-[8-methoxy-4-(5-methyl-2H-pyrazol-3-ylamino)-quinazolin-2-yl]-2,5-diaza-bicyclo[2.2.1]hept-2-yl}-ethanone;
[2-((1S,4S)-5-Methanesulfonyl-2,5-diaza-bicyclo[2.2.1]hept-2-yl)-8-methoxy-quinazolin-4-yl]-(5-methyl-2H-pyrazol-3-yl)-amine;
(1S,4S)-5-[8-Methoxy-4-(5-methyl-2H-pyrazol-3-ylamino)-quinazolin-2-yl]-2,5-diaza-bicyclo[2.2.1]heptane-2-carboxylic acid methyl ester;
{(1R,5S)-3-[8-Methoxy-4-(5-methyl-1H-pyrazol-3-ylamino)-quinazolin-2-yl]-3-aza-bicyclo[3.1.0]hex-6-yl}-carbamic acid tert-butyl ester;
{(1R,5S)-3-[8-Methoxy-4-(5-methyl-1H-pyrazol-3-ylamino)-quinazolin-2-yl]-3-aza-bicyclo[3.1.0]hex-1-yl}-carbamic acid tert-butyl ester; and the pharmaceutically acceptable salts and solvates of the foregoing compounds.

Specific preferred embodiments of the compounds of Formula I include those selected from the group consisting of:
N4-(5-Methyl-1H-pyrazol-3-yl)-N2-[(1R,5S,6S)-3-(propane-2-sulfonyl)-3-aza-bicyclo[3.1.0]hex-6-yl]-pyrimidine-2,4-diamine;
N4-(5-Methyl-1H-pyrazol-3-yl)-N2-[(1S,5R)-3-(propane-1-sulfonyl)-3-aza-bicyclo[3.1.0]hex-6-yl]-pyrimidine-2,4-diamine;
3-{(1R,5S)-6-[4-(5-Methyl-1H-pyrazol-3-ylamino)-pyrimidin-2-ylamino]-3-aza-bicyclo[3.1.0]hexane-3-sulfonyl}-benzonitrile;
3-Cyano-N-{(1R,5S)-3-[4-(5-methyl-1H-pyrazol-3-ylamino)-pyrimidin-2-yl]-3-aza-bicyclo[3.1.0]hex-6-yl}-benzenesulfonamide;

N2-[(1R,5S)-3-(3,5-Dimethyl-1H-pyrazole-4-sulfonyl)-3-aza-bicyclo[3.1.0]hex-6-yl]-N4-(5-methyl-1H-pyrazol-3-yl)-pyrimidine-2,4-diamine;
(1R,5S)-6-[4-(5-Methyl-1H-pyrazol-3-ylamino)-pyrimidin-2-ylamino]-3-aza-bicyclo[3.1.0]hexane-3-carboxylic acid (1H-indol-3-yl)-amide;
(1R,5S)-6-[4-(5-Methyl-1H-pyrazol-3-ylamino)-pyrimidin-2-ylamino]-3-aza-bicyclo[3.1.0]hexane-3-carboxylic acid ((R)-1-phenyl-ethyl)-amide; and the pharmaceutically acceptable salts and solvates of the foregoing compounds.

The invention also provides for a method of preparing a compound of Formula I which comprises reacting a compound of the Formula II

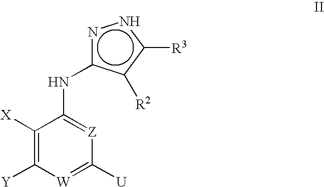

wherein U is a leaving group and W, X, Y, R², and R³ are as defined above with a compound of the formula V—R¹, wherein V, and R¹ are as defined above.

In a preferred embodiment of the method of the present invention U is a halo and preferably a Cl.

This invention also relates to a method for the treatment of abnormal cell growth in a mammal, including a human, comprising administering to said mammal an amount of a compound of the Formula I, as defined above, or a pharmaceutically acceptable salt or solvate thereof, that is effective in treating abnormal cell growth.

In one embodiment of this method, the abnormal cell growth is cancer, Including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In a preferred embodiment of the present invention the cancer is selected from lung cancer (NSCLC and SCLC), cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non hodgkins's lymphoma, spinal axis tumors, or a combination of one or more of the foregoing cancers.

In another preferred embodiment of the present invention the cancer is selected from lung cancer (NSCLC and SCLC), ovarian cancer, colon cancer, rectal cancer, cancer of the anal region, or a combination of one or more of the foregoing cancers.

In a more preferred embodiment of the present invention the cancer is selected from lung cancer (NSCLC and SCLC), ovarian cancer, colon cancer, rectal cancer, or a combination of one or more of the foregoing cancers.

In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a compound of Formula I, or a pharmaceutically acceptable salt or solvate thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of a compound of the Formula I, as defined above, or a pharmaceutically acceptable salt or solvate thereof, that is effective in treating abnormal cell growth, and a pharmaceutically acceptable carrier. In one embodiment of said composition, said abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

The invention also relates to a pharmaceutical composition for the treatment of abnormal cell growth in a mammal, including a human, which comprises an amount of a compound of Formula I, as defined above, or a pharmaceutically acceptable salt or solvate thereof, that is effective in treating abnormal cell growth in combination with a pharmaceutically acceptable carrier and an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The invention also relates to a method for the treatment of a hyperproliferative disorder in a mammal which comprises administering to said mammal a therapeutically effective amount of a compound of Formula I, or a pharmaceutically acceptable salt or hydrate thereof, in combination with an anti-tumor agent selected from the group consisting antiproliferative agents, kinase inhibitors, angiogenesis inhibitors, growth factor inhibitors, cox-I inhibitors, cox-II inhibitors, mitotic inhibitors, alkylating agents, anti-metabolites, Intercalating antibiotics, growth factor inhibitors, radiation, cell cycle inhibitors, enzymes, topoisomerase inhibitors, biological response modifiers, antibodies, cytotoxics, anti-hormones, statins, and anti-androgens.

In one embodiment of the present invention the anti-tumor agent used in conjunction with a compound of Formula I and pharmaceutical compositions described herein is an anti-angiogenesis agent, kinase inhibitor, pan kinase inhibitor or growth factor inhibitor.

Preferred pan kinase inhibitors include SU-11248, described in U.S. Pat. No. 6,573,293 (Pfizer, Inc, NY, USA).

Anti-angiogenesis agents, include but are not limited to the following agents, such as EGF inhibitor, EGFR inhibitors, VEGF inhibitors, VEGFR inhibitors, TIE2 inhibitors, IGF1R inhibitors, COX-II (cyclooxygenase II) inhibitors, MMP-2 (matrix-metalloproteinase 2) inhibitors, and MMP-9 (matrix-metalloproteinase 9) inhibitors.

Preferred VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc., NY, USA), AG13736 (Pfizer Inc.), ZD-6474 (AstraZeneca), AEE788 (Novartis), AZD-2171), VEGF Trap (Regeneron/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. VEGF Inhibitors useful in the practice of the present invention are disclosed in U.S. Pat. Nos. 6,534,524 and 6,235,764, both of which are incorporated in their entirety for all purposed.

Particularly preferred VEGF inhibitors include CP-547,632, AG13736, Vatalanib, Macugen and combinations thereof.

Additional VEGF inhibitors are described in, for example in WO 99/24440 (published May 20, 1999), PCT International Application PCT/IB99/00797 (filed May 3, 1999), In WO 95/21613 (published Aug. 17, 1995), WO 99/61422 (published Dec. 2, 1999), U.S. Pat. No. 6,534,524 (discloses AG13736), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. patent application Ser. Nos. 09/221,946 (filed Dec. 28, 1998); 09/454,058 (filed Dec. 2, 1999); 09/501,163 (filed Feb. 9, 2000); 09/539,930 (filed Mar. 31, 2000); 09/202,796 (filed May 22, 1997); 09/384,339 (filed Aug. 26, 1999); and 09/383,755 (filed Aug. 26, 1999); and the compounds disclosed and claimed in the following U.S. provisional patent application Ser. Nos. 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

PDGRr inhibitors include but not limited to those disclosed international patent application publication number WO01/40217, published Jul. 7, 2001 and international patent application publication number WO2004/020431, published Mar. 11, 2004, the contents of which are incorporated in their entirety for all purposes.

Preferred PDGFr inhibitors include Pfizer's CP-673,451 and CP-868,596 and its pharmaceutically acceptable salts.

Preferred GARF inhibitors include Pfizer's AG-2037 (pelitrexol and its pharmaceutically acceptable salts. GARF inhibitors useful in the practice of the present invention are disclosed in U.S. Pat. No. 5,608,082 which is incorporated in its entirety for all purposed.

Examples of useful COX-II inhibitors which can be used in conjunction with a compound of Formula I and pharmaceutical compositions described herein include CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-II inhibitors are disclosed in U.S. patent application Ser. Nos. 10/801,446 and 10/801,429, the contents of which are incorporated in their entirety for all purposes.

In one preferred embodiment the anti-tumor agent is celecoxib as disclosed in U.S. Pat. No. 5,466,823, the contents of which are incorporated by reference in its entirety for all purposes. The structure for Celecoxib is shown below:

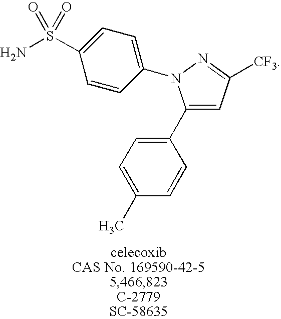

In one preferred embodiment the anti-tumor agent is valecoxib as disclosed in U.S. Pat. No. 5,633,272, the contents of which are incorporated by reference in its entirety for all purposes. The structure for valdecoxib is shown below:

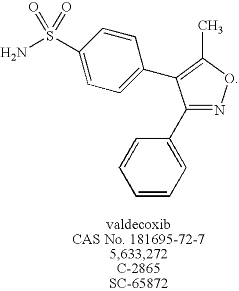

In one preferred embodiment the anti-tumor agent is parecoxib as disclosed in U.S. Pat. No. 5,932,598, the contents of which are incorporated by reference in its entirety for all purposes. The structure for paracoxib is shown below:

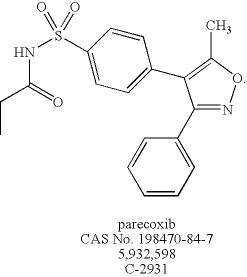

In one preferred embodiment the anti-tumor agent is deracoxib as disclosed in U.S. Pat. No. 5,521,207, the contents of which are incorporated by reference in its entirety for all purposes. The structure for deracoxib is shown below:

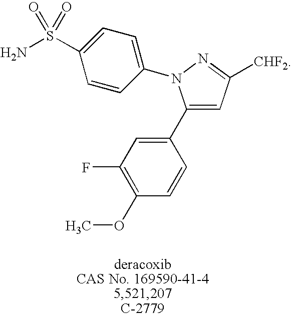

In one preferred embodiment the anti-tumor agent is SD-8381 as disclosed in U.S. Pat. No. 6,034,256, the contents of which are incorporated by reference in its entirety for all purposes. The structure for SD-8381 is shown below:

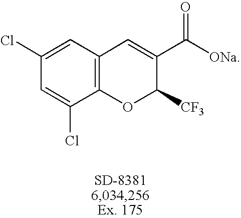

In one preferred embodiment the anti-tumor agent is ABT-963 as disclosed in International Publication Number WO 2002/24719, the contents of which are incorporated by reference in its entirety for all purposes. The structure for ABT-963 is shown below:

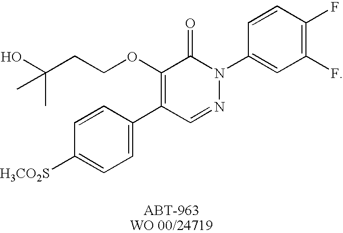

In one preferred embodiment the anti-tumor agent is rofecoxib as shown below:

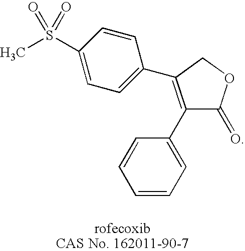

In one preferred embodiment the anti-tumor agent is MK-663 (etoricoxib) as disclosed in international Publication Number WO 1998/03484, the contents of which are incorporated by reference in its entirety for all purposes. The structure for etoricoxib is shown below:

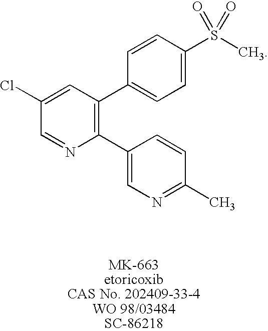

In one preferred embodiment the anti-tumor agent is COX-189 (Lumiracoxib) as disclosed in international Publication Number WO 1999/11605, the contents of which are incorporated by reference in its entirety for all purposes. The structure for Lumiracoxib is shown below:

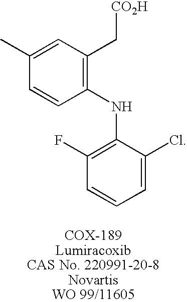

In one preferred embodiment me anti-tumor agent is BMS-347070 as disclosed in U.S. Pat. No. 6,180,651, the contents of which are incorporated by reference in its entirety for all purposes. The structure for BMS-347070 is shown below:

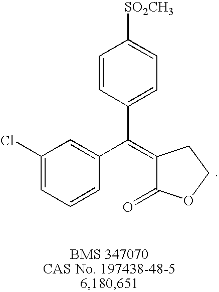

In one preferred embodiment the anti-tumor agent is NS-398 (CAS123653-11-2). The structure for NS-398 (CAS123653-11-2) is shown below:

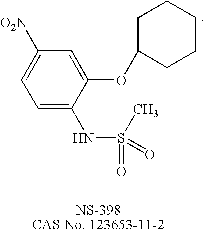

In one preferred embodiment the anti-tumor agent is RS 57067 (CAS17932-91-3). The structure for RS-57067 (CAS17932-91-3) is shown below:

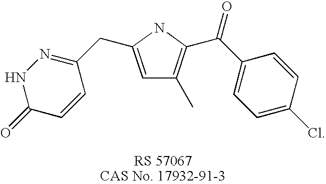

In one preferred embodiment the anti-tumor agent is 4-Methyl-2-(3,4-dimethylphenyl)-1-(4-sulfamoyl-phenyl)-1H-pyrrole. The structure for 4-Methyl-2-(3,4-dimethylphenyl)-1-(4-sulfamoyl-phenyl)-1H-pyrrole is shown below:

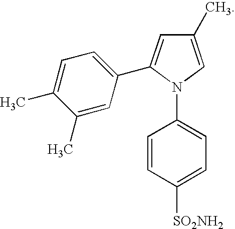

In one preferred embodiment the anti-tumor agent is 2-(4-Ethoxyphenyl)-4-methyl-1-(4-sulfamoylphenyl)-1H-pyrrole. The structure for 2-(4-Ethoxyphenyl)-4-methyl-1-(4-sulfamoylphenyl)-1H-pyrrole is shown below:

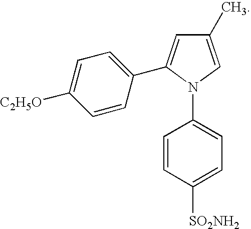

In one preferred embodiment the anti-tumor agent is meloxicam. The structure for meloxicam is shown below:

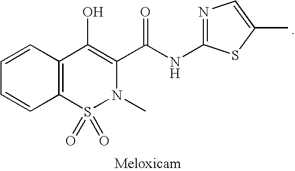

Other useful inhibitors as anti-tumor agents used in conjunction with a compound of Formula I and pharmaceutical compositions described herein include aspirin, and non-steroidal anti-inflammatory drugs (NSAIDs) which inhibit the enzyme that makes prostaglandins (cyclooxygenase I and II), resulting in lower levels of prostaglandins, include but are not limited to the following, Salsalate (Amigesic), Diflunisal (Dolobld), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred COX-I inhibitors include ibuprofen (Motrin), nuprin, naproxen (Aleve), indomethacin (Indocin), nabumetone (Relafen) and combinations thereof.

Targeted agents used in conjunction with a compound of Formula I and pharmaceutical compositions described herein include EGFr inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof.

Preferred EGFr inhibitors include Iressa, Erbitux, Tarceva and combinations thereof.

The present invention also relates to anti-tumor agents selected from pan erb receptor inhibitors or ErbB2 receptor inhibitors, such as CP-724,714 (Pfizer, Inc.), CI-1033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafarnib, GlaxoSmithKline), GW-282974 (GlaxoSmithKline), EKB-569 (Wyeth), PKI-166 (Novartis), dHER2 (HER2Vaccine, Corixa and GlaxoSmithKline), APC8024 (HER2Vaccine, Dendreon), anti-HER2/neu bispecific antibody (Decof Cancer Center), B7.her2.IgG3 (Agensys), AS HER2 (Research institute for Rad Biology & Medicine), trifunctional bispecific antibodies (University of Munich) and mAB AR-209 (Aronex Pharmaceuticals Inc) and mAB 2B-1 (Chiron) and combinations thereof.

Preferred erb selective anti-tumor agents include Herceptin, TAK-165, CP-724,714, ABX-EGF, HER3 and combinations thereof.

Preferred pan erbb receptor inhibitors include GW572016, CI-1033, EKB-569, and Omitarg and combinations thereof.

Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (Issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277 each of which is herein incorporated by reference in its entirety.

Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aetema), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (paditaxel), Evista (raloxifene), Atamestane (Biomed-777), Xyotax (polyglutamate paclitaxel), Targetin (bexarotine) and combinations thereof.

Additionally, other anti-tumor agents may be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof.

Further anti-tumor agents may selected from the following agents, CeaVac (CEA), NeuTrexin (trimetresate glucuronate) and combinations thereof.

Additional anti-tumor agents may selected from the following agents, OvaRex (oregovomab), Osidem (IDM-1), and combinations thereof.

Additional anti-tumor agents may selected from the following agents, Advexin (ING 201), Tirazone (tirapazamine), and combinations thereof.

Additional anti-tumor agents may selected from the following agents, RSR13 (efaproxiral), Cotara (131I chTNT 1/b), NBI-3001 (IL-4) and combinations thereof.

Additional anti-tumor agents may selected from the following agents, Canvaxin, GMK vaccine, Oncophage (HSPPC-96), PEG Interon A, Taxoprexin (DHA/paciltaxel) and combinations thereof.

Other preferred anti-tumor agents include Pfizer's MEK1/2 inhibitor PD325901, Array Biopharm's MEK inhibitor ARRY-142886, Bristol Myers' CDK2 inhibitor BMS-387,032, Pfizer's CDK inhibitor PD0332991 and AstraZeneca's AXD-5438 and combinations thereof.

Additionally, mTOR inhibitors may also be utilized such as CCI-779 (Wyeth) and rapamycin derivatives RAD001 (Novartis) and AP-23573 (Ariad), HDAC inhibitors SAHA (Merck Inc./Aton Pharmaceuticals) and combinations thereof.

Additional anti-tumor agents include aurora 2 inhibitor VX-680 (Vertex), Chk1/2 inhibitor XL844 (Exilixis).

The following cytotoxic agents, e.g., one or more selected from the group consisting of epirubicin (Ellence), docetaxel (Taxotere), paclitaxel, Zinecard (dexrazoxane), rituximab (Rituxan) imatinib mesylate (Gleevec), and combinations thereof, may be used in conjunction with a compound of Formula I and pharmaceutical compositions described herein.

The invention also contemplates the use of the compounds of the present invention together with hormonal therapy, Including but not limited to, exemestane (Aromasin, Pfizer Inc.), leuprorelin (Lupron or Leuplin, TAP/Abbott/Takeda), anastrozole (Arimidex, Astrazeneca), gosrelin (Zoladex, AstraZeneca), doxercalciferol, fadrozole, formestane, tamoxifen citrate (tamoxifen, Nolvadex, AstraZeneca), Casodex (AstraZeneca), Abarelix (Praecis), Trelstar, and combinations thereof.

The invention also relates to hormonal therapy agents such as anti-estrogens including, but not limited to fulvestrant, toremifene, raloxifene, lasofoxifene, letrozole (Femara, Novartis), anti-androgens such as bicalutamide, flutamide, mifepristone, nilutamide, Casodex® (4′-cyano-3-(4-fluorophenylsulphonyl)-2-hydroxy-2-methyl-3′-(trifluoromethyl) propionanilide, bicalutamide) and combinations thereof.

Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

Particularly preferred cytotoxic agents include Camptosar, Erbitux, Iressa, Gleevec, Taxotere and combinations thereof.

The following topoisomerase I inhibitors may be utilized as anti-tumor agents camptothecin, irinotecan HCl (Camptosar), edotecarin, orathecin (Supergen), exatecan (Daiichi), BN-80915 (Roche) and combinations thereof.

Particularly preferred toposimerase II inhibitors include epirubicin (Ellence).

The compounds of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers.

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, Paraplatin (carboplatin), eptaplatin, lobaplatin, nedaplatin, Eloxatin (oxaliplatin, Sanofi) or satrplatin and combinations thereof.

Particularly preferred alkylating agents include Eloxatin (oxaliplatin).

Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, Allmta (premetrexed disodium, LY231514, MTA), Gemzar (gemcitabine, Eli Lilly), fludarabin, 5-azacitidine, capecitabine, cladribine, clofarabine, decitabine, eflomithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

Antibiotics include intercalating antibiotics but are not limited to: aclarubicin, actinomycin D, amrubicin, annamycin, adriamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin, zinostatin and combinations thereof.

Plant derived anti-tumor substances include for example those selected from mitotic inhibitors, for example vinblastine, docetaxel (Taxotere), paclitaxel and combinations thereof.

Cytotoxic topoisomerase inhibiting agents include one or more agents selected from the group consisting of aclarubicin, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, topotecan, and combinations thereof.

Preferred cytotoxic topoisomerase inhibiting agents include one or more agents selected from the group consisting of camptothecin, 10-hydroxycamptothecin, 9-aminocamptothecin, irinotecan HCl (Camptosar), edotecarin, epirubicin (Ellence), etoposide, SN-38, topotecan, and combinations thereof.

Immunologicals include interferons and numerous other immune enhancing agents. Interferons include interferon alpha, interferon alpha-2a, Interferon, alpha-2b, interferon beta, interferon gamma-1a, interferon gamma-1b (Actimmune), or interferon gamma-n1 and combinations thereof. Other agents include filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab (Y-muHMFG1), Provenge (Dendreon) and combinations thereof.

Biological response modifiers are agents that modify defense mechanisms of living organisms or biological responses, such as survival, growth, or differentiation of tissue cells to direct them to have anti-tumor activity. Such agents include krestin, lentinan, sizofuran, picibanil, ubenimex and combinations thereof.

Other anticancer agents include alitretinoin, ampligen, atrasentan bexarotene, bortezomib. Bosentan, calcitriol, exisulind, finasteride, fotemustine, ibandronic acid, miltefosine, mitoxantrone, 1-asparaginase, procarbazine, dacarbazine, hydroxycarbamide, pegaspargase, pentostatin, tazarotne, Telcyta (TLK-286, Telik Inc.), Velcade (bortemazib, Millenium), tretinoin, and combinations thereof.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof.

Platinum-coordinated compounds include but are not limited to, cisplatin, carboplatin, nedaplatin, oxaliplatin, and combinations thereof.

Camptothecin derivatives include but are not limited to camptothecin, 10-hydroxycamptothecin, 9-aminocamptothecin, irinotecan, SN-38, edotecarin, topotecan and combinations thereof.

Other antitumor agents include mitoxantrone, 1-asparaginase, procarbazine, dacarbazine, hydroxycarbamide, pentostatin, tretinoin and combinations thereof.

Anti-tumor agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736; and anti-proliferative agents such as other farnesyl protein transferase inhibitors, for example the farnesyl protein transferase inhibitors. Additional, specific CTLA4 antibodies that can be used in the present invention include those described in U.S. Provisional Application 60/113,647 (filed Dec. 23, 1998), U.S. Pat. No. 6,682,736 both of which are herein incorporated by reference in their entirety.

Specific IGF1R antibodies that can be used in the present invention include those described in International Patent Application No. WO 2002/053596, which is herein incorporated by reference in its entirety.

Specific CD40 antibodies that can be used in the present invention include those described in International Patent Application No. WO 2003/040170 which is herein incorporated by reference in its entirety.

Gene therapy agents may also be employed as anti-tumor agents such as TNFerade (GeneVec), which express TNFalpha in response to radiotherapy.

In one embodiment of the present invention statins may be used in conjunction with a compound of Formula I and pharmaceutical compositions. Statins (HMG-CoA reducatase inhibitors) may be selected from the group consisting of Atorvastatin (Lipitor, Pfizer Inc.), Provastatin (Pravachol, Bristol-Myers Squibb), Lovastatin (Mevacor, Merck Inc.), Simvastatln (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof.

In a preferred embodiment the statin is selected from the group consisting of Atovorstatin and Lovastatin, derivatives and combinations thereof.

Other agents useful as anti-tumor agents include Caduet.

In one preferred embodiment radiation can be used in conjunction with a compound of Formula I and pharmaceutical compositions described herein. Radiation may be administered in a variety of fashions. For example, radiation may be electromagnetic or particulate in nature. Electromagnetic radiation useful in the practice of this invention includes, but is not limited, to x-rays and gamma rays. In a preferable embodiment, supervoltage x-rays α-rays>=4 MeV) may be used in the practice of this invention. Particulate radiation useful in the practice of this invention includes, but is not limited to, electron beams, protons beams, neutron beams, alpha particles, and negative pi mesons. The radiation may be delivered using conventional radiological treatment apparatus and methods, and by intraoperative and stereotactic methods. Additional discussion regarding radiation treatments suitable for use in the practice of this invention may be found throughout Steven A. Leibel et al., Textbook of Radiation Oncology (1998) (publ. W. B. Saunders Company), and particularly in Chapters 13 and 14. Radiation may also be delivered by other methods such as targeted delivery, for example by radioactive “seeds,” or by systemic delivery of targeted radioactive conjugates. J. Padawer et al., Combined Treatment with Radioestradiol Iucanthone in Mouse C3HBA Mammary Adenocarcinoma and with Estradlol Iucanthone in an Estrogen Bioassay, Int. J. Radiat. Oncol. Biol. Phys. 7:347-357 (1981). Other radiation delivery methods may be used in the practice of this invention.

The amount of radiation delivered to the desired treatment volume may be variable. In a preferable embodiment, radiation may be administered in amount effective to cause the arrest or regression of the cancer, in combination with a compound of Formula I and pharmaceutical compositions described herein.

In a more preferable embodiment, radiation is administered in at least about 1 Gray (Gy) fractions at least once every other day to a treatment volume, still more preferably radiation is administered in at least about 2 Gray (Gy) fractions at least once per day to a treatment volume, even more preferably radiation is administered in at least about 2 Gray (Gy) fractions at least once per day to a treatment volume for five consecutive days per week.

In a more preferable embodiment, radiation is administered in 3 Gy fractions every other day, three times per week to a treatment volume.

In yet another more preferable embodiment, a total of at least about 20 Gy, still more preferably at least about 30 Gy, most preferably at least about 60 Gy of radiation is administered to a host in need thereof.

In one more preferred embodiment of the present invention 14 GY radiation is administered.

In another more preferred embodiment of the present invention 10 GY radiation is administered.

In another more preferred embodiment of the present invention 7 GY radiation is administered.

In a most preferable embodiment, radiation is administered to the whole brain of a host, wherein the host is being treated for metastatic cancer.

Examples of useful matrix metalloproteinase inhibitors used in conjunction with a compound of Formula I and pharmaceutical compositions described herein are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety.

Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the compounds recited in the following list:
3-[[4-(4-fluoro-phenoxy)-benzenesulfonyl]-(1-hydroxycarbamoyl-cyclopentyl)-amino]-propionic acid;
3-exo-3-[4-(4-fluoro-phenoxy)-benzenesulfonylamino]-8-oxa-bicyclo[3.2.1]octane-3-carboxylic acid hydroxyamide;
(2R,3R) 1-[4-(2-chloro-4-fluoro-benzyloxy)-benzenesulfonyl]-3-hydroxy-3-methyl-piperidine-2-carboxylic acid hydroxyamide;
4-[4-(4-fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
3-[4-(4-chloro-phenoxy)-benzenesulfonylamino]-tetrahydro-pyran-3-carboxylic acid hydroxyamide;
(2R,3R) 1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-fluoro-phenoxy)-benzenesulfonyl]-(4-hydroxycarbamoyl-tetrahydro-pyran-4-yl)-amino]-propionic acid;
3-exo-3-[4-(4-chloro-phenoxy)-benzenesulfonylamino]-8-oxa-bicyclo[3.2.1]octane-3-carboxylic acid hydroxyamide;
3-endo-3-[4-(4-fluoro-phenoxy)-benzenesulfonylamino]-8-oxa-bicyclo[3.2.1]octane-3-carboxylic acid hydroxyamide; and
3-[4-(4-fluoro-phenoxy)-benzenesulfonylamino]-tetrahydro-furan-3-carboxylic acid hydroxyamide;

and pharmaceutically acceptable salts, solvates and prodrugs of said compounds.

Various other compounds, such as styrene derivatives, have also been shown to possess tyrosine kinase inhibitory properties, and some of tyrosine kinase inhibitors have been identified as erbB2 receptor inhibitors. More recently, five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are World Patent Application WO00/44728 (published Aug. 3, 2000), EP 1029853A1 (published Aug. 23, 2000), and WO01/98277 (published Dec. 12, 2001) all of which are incorporated herein by reference in their entirety.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; and (4) any tumors that proliferate by receptor tyrosine kinases.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “aliphatic” as used herein means straight-chain, branched or cyclic (C₁-C₁₂) hydrocarbons which are completely saturated or which contain one or more units of unsaturation but which are not aromatic.

For example, suitable aliphatic groups include substituted or unsubstituted linear, branched or cyclic alkyl, alkenyl, alkynyl groups and hybrids thereof such as (cycloalkyl)alkyl, (cycloalkenyl)alkyl or (cycloalkyl)alkenyl.

The term “alkyl”, as used herein means saturated monovalent hydrocarbon radicals having straight, branched, or cyclic moieties (including fused and bridged bicyclic and spirocyclic moieties), or a combination of the foregoing moieties. For an alkyl group to have cyclic moieties, the group must have at least three carbon atoms.

The term “alkoxy”, as used herein means O-alkyl groups wherein alkyl is as defined above.

The terms “hydroxyalkyl”, “alkoxyalkyl”, and alkoxycarbonyl”, used alone or as part of a larger moiety includes both straight and branched chains containing one to twelve carbon atoms.

The term “alkenyl” used alone or as part of a larger moiety shall include both straight and branched chains containing two to twelve carbon atoms having at least one carbon-carbon double bond. The terms “alkynyl” used alone or as part of a larger moiety shall include both straight and branched chains containing two to twelve carbon atoms having at least one carbon-carbon triple bond. The term “cycloalkyl used alone or as part of a larger moiety shall include cyclic (C₃-C₁₂) hydrocarbons which are completely saturated or which contain one or more units of unsaturation, but which are not aromatic.

The terms “haloalkyl”, “haloalkenyl” and haloalkoxy” means alkyl, alkenyl or alkoxy, as the case may be, substituted with one or more halogen atoms. The term “halo” is used herein interchangeably with the term “halogen” means F, Cl, Br, or I. Preferred halo groups are F, Cl, and Br.

The term “heteroatom”, means nitrogen, oxygen, or sulfur and includes any oxidized form of nitrogen and sulfur, and the quaternized form of any basic nitrogen. Also the term “nitrogen” includes a substitutable nitrogen of a heterocyclic ring. As an example, in a saturated or partially unsaturated ring having 0 to 3 heteroatoms selected from oxygen, sulfur or nitrogen, the nitrogen may be N (as in 3,4-dihydro-2H-pyrrolyl), NH (as in pyrrolidinyl) or NOR (as in N-substituted pyrrolidinyl). The terms “carbocycle”, “carbocyclyl”, “carbocyclo”, or “carbocyclic” as used herein means an aliphatic ring system having three to fourteen members. The terms “carbocycle”, “carbocyclyl”, “carbocyclo”, or “carbocyclic” whether saturated or partially unsaturated, also refers to rings that are optionally substituted. The terms “carbocycle”, “carbocyclyl,” “carbocyclo”, or “carbocyclic” also include aliphatic rings that are fused to one or more aromatic or non-aromatic rings, such as in a decahydronaphthyl or tetrahydronaphthyl, where the radical or point-of attachment is on the aliphatic ring.

The term “aryl” used alone or as part of a larger moiety as in “aralkyl”, “aralkoxy”, or “aryloxyalkyl”, refers to aromatic ring groups having five to fourteen members, such as phenyl, benzyl, phenethyl, 1-naphthyl, 2-naphthyl, 1-anthracyl and 2-anthracyl. The term “aryl” also refers to rings that are optionally substituted. The term “aryl” may be used interchangeably with the term aryl ring. “Aryl” also includes fused polycyclic aromatic ring systems in which an aromatic ring is fused to one or more rings. Examples include 1-naphthyl, 2-naphthyl, 1-anthracyl and 2-anthracyl. Also included within the scope of the term “aryl” as it is used herein, is a group in which an aromatic ring is fused to one or more non-aromatic rings, such as in an indanyl, phenanthridinyl, or tetrahydronaphthyl, where the radical or point of attachment is on the aromatic ring.

The term “heterocycle”, “heterocyclyl”, or “heterocyclic” as used herein includes non-aromatic ring systems having four to fourteen members, preferably five to ten, in which one or more ring carbons, preferably one to four, are each replaced by a heteroatom such as N, O, or S. Non-aromatic heterocyclic groups include groups having only 4 atoms in their ring system, but aromatic heterocyclic groups must have at least 5 atoms in their ring system. The heterocyclic groups include benzo-fused ring systems. Examples of heterocyclic rings include 3-1H-benzimidazol-2-one, (1-substituted)-2-oxo-benzimidazol-3-yl, 2-tetrahydrofuranyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term heterocyclyl”, or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocycle”, “heterocyclyl”, or “heterocyclic” whether saturated or partially unsaturated, also refers to rings that are optionally substituted.

An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl.

Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl.

Also included within the scope of the term “heteroaryl”, as it is used herein, is a group in which a heteroatomic ring is fused to one or more aromatic or nonaromatic rings where the radical or point of attachment is on the heteroaromatic ring. Examples include tetrahydroquinolinyl, tetrahydroisoquinolinyl, and pyrido[3,4-d]pyrimidinyl.

The term “heteroaryl”, used alone or as part of a larger moiety as in “heteroaralkyl” or “heteroarylalkoxy”, refers to heteroaromatic ring groups having five to fourteen members. Examples of heteroaryl rings include 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2 5 pyrazolyl, 3-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, Indazolyl, isoindolyl, acridinyl, or benzoisoxazolyl.

The term “heteroaryl” also refers to rings that are optionally substituted. The term heteroaryl” may be used interchangeably with the term “heteroaryl ring” or the term “heteroaromatic”. An aryl (including aralkyl, aralkoxy, aryloxyalkyl and the like) or heteroaryl (including heteroaralkyl and heteroarylalkoxy and the like) group may contain one or more substituents.

The phrase “pharmaceutically acceptable salt(s)”, as used herein, unless otherwise indicated, includes salts of acidic or basic groups which may be present in the compounds of Formula I. The compounds of Formula I that are basic in nature are capable of forming a wide variety of salts with various inorganic and organic acids. The acids that may be used to prepare pharmaceutically acceptable acid addition salts of such basic compounds of Formula I are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, triethiodode, and valerate salts.

In the compounds of Formula I, where terms such as (CR¹R²)_{q} or (CR¹R²)ₜ are used, R¹ and R² may vary with each iteration of q or t above 1. For instance, where q or t is 2 the terms (CR¹R²)_{q} or (CR¹R²)ₜ may equal —CH₂ CH₂—, or —CH(CH₃)C(CH₂ CH₃)(CH₂ CH₂ CH₃)—, or any number of similar moieties falling within the scope of the definitions of R¹ and R². Further, as noted above, any substituents comprising a CH₃ (methyl), CH₂ (methylene), or CH (methine) group which is not attached to a halogen, SO or SO₂ group or to a N, O or S atom optionally bears on said group a substituent selected from hydroxy, C₁-C₄ alkoxy and —NR¹R².

Certain compounds of Formula I may have asymmetric centers and therefore exist in different enantiomeric forms. All optical isomers and stereoisomers of the compounds of Formula I, and mixtures thereof, are considered to be within the scope of the invention. With respect to the compounds of Formula I, the invention includes the use of a racemate, one or more enantiomeric forms, one or more diastereomeric forms, or mixtures thereof. The compounds of Formula I may also exist as tautomers. This invention relates to the use of all such tautomers and mixtures thereof.

The subject invention also includes isotopically-labelled compounds, which are identical to those recited in Formula I, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁸Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

### DETAILED DESCRIPTION OF THE INVENTION

General synthetic methods which may be referred to for preparing the compounds of the present invention are provided in published international patent applications: WO 2002/022601, WO 2002/022602, WO 2002/022603, WO 2002/022604, WO 2002/022605, WO 2002/022606, WO 2002/022607, WO 2002/022608, WO 2002/50066, WO 2002/068415, WO 2002/066461, WO 2002/050065, WO 2002/096905, WO 2004/000833, WO 2002/066461, WO 2002/068415, WO 2001/021594, WO 2001/055116, WO2001/021596, and WO 2001/021597 all of the foregoing published patent applications are incorporated herein by reference in their entirety. Certain starting materials may be prepared according to methods familiar to those skilled in the art and certain synthetic modifications may be done according to methods familiar to those skilled in the art. Fused and bridged bicyclic amines were synthesized according to the methods described in: Brighty, K. E. and Castaldi, M. J., Synlett, 1996, 1097 and WO 2004/074292. Starting materials, the synthesis of which is not specifically described herein or the published references referred to above, are either commercially available or can be prepared using methods well known to those of skill in the art.

The general synthetic scheme used to prepare compounds of this invention is outlined in Scheme 1 below for Methods A to F.

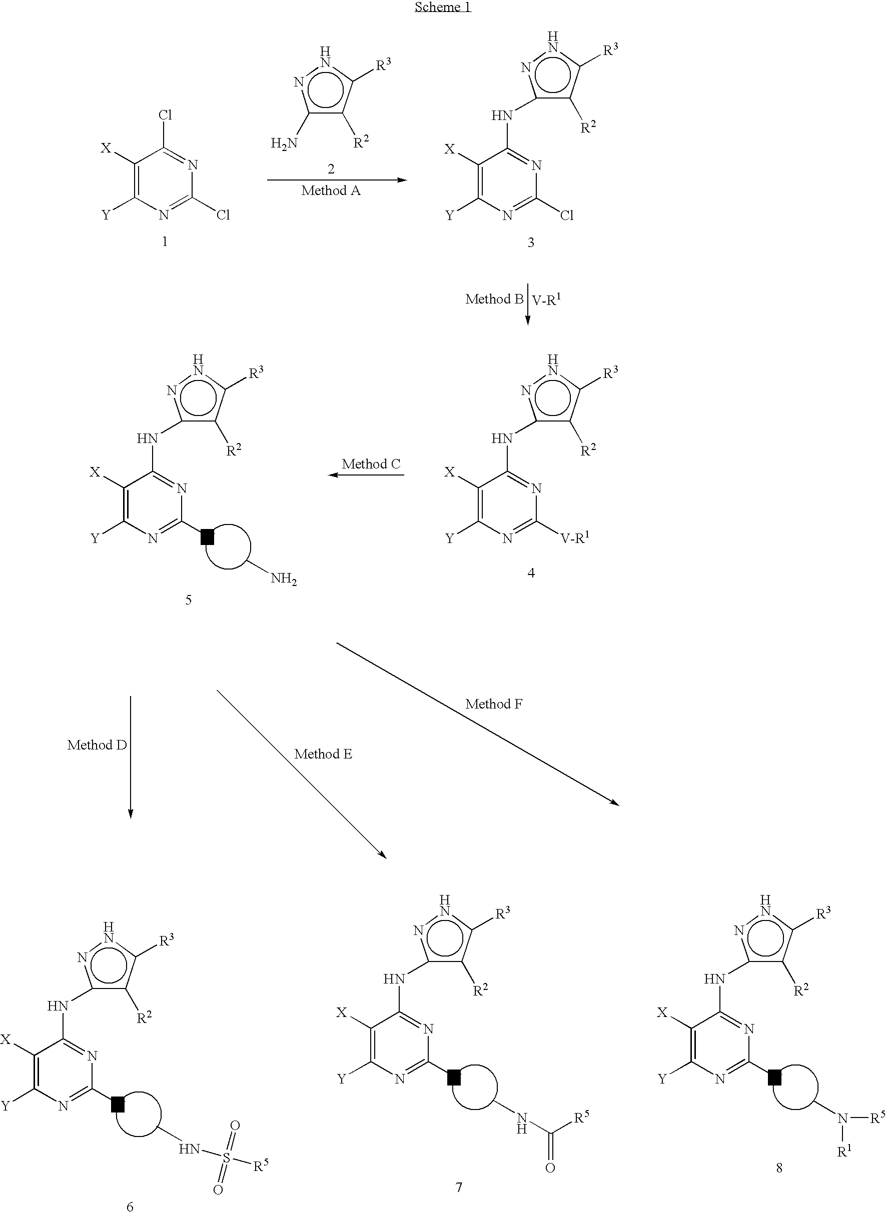

The substituents X, Y, V, R², R³, and R⁵ shown in Scheme 1 are as defined above in the Summary of the invention.

Preparation of the starting 2,4-dichloropyrimidine derivative of Formula 1 may be achieved in a manner similar to that described in Chem. Pharm. Bull., 30, 9, 1982, 3121-3124.

Method A: The nucleophilic displacement of the 4-chloro substituent of the compound of Formula 1 with a nucleophilic center is well-precedented in the literature. This displacement can be achieved utilizing ring substituted 3-amino-pyrazole derivatives (Formula 2) in a manner similar to that described in J. Med. Chem., 38, 1995, 3547-3557 to give an intermediate of the Formula 3. This method is illustrated in Examples 1 and 2 below.

Method B: Subsequent displacement of the 2-chloro group of the compound of Formula 3 may be carried out in a manner similar to that described in J. Med. Chem., 38, 1995, 2763-2773 and J. Chem. Soc., 1948, 1766-1771 to give a compound of Formula 4. This displacement may be carried out with amino-cycloalkanes or amino-bicycloalkanes or amino-aza-bicycloalkanes. This method is illustrated in Examples 1 to 6 below.

Method C: The compound of Formula 4 may result from reaction of a carbamate protected amino-aza-bicycloalkane with a compound of Formula 3. It is well known in the art that a t-butyl carbamate may be removed under acid catalysis (Green, Wuts, Protective Groups in Organic Synthesis, Third Edition, pp 518-525) to provide the corresponding unprotected amine as the conjugate acid. Likewise, it is known that a benzyloxy carbamate (CBZ) can be removed under a variety of conditions (Green, Wuts, Protective Groups in Organic Synthesis, Third Edition, pp 531-537). The application of a carbamate deprotection protocol to a suitably substituted 2-aminopyrimidine of the Formula 4 to afford an amine compound of the Formula 5 is shown in Examples 1 to 5 below.

Method D: It may be desirable to further derivatize an unprotected amine of the compound of Formula 5 with substituted carbonyl, substituted sulfonyl, or substituted alkyl groups to create compounds of interest. The preparation of a sulfonamide via reaction of a primary or secondary amine with a sulfonyl halide or anhydride in the presence of an organic or inorganic base is a transformation well documented in the art. A representative example of this method to produce substituted sulfonyl derivative compound of the Formula 6 is shown in Example 1 below.

Method E: It may be desirable to further derivatize the unprotected amine of Formula 5 with substituted carbonyl or substituted alkyl groups to create compounds of interest. The preparation of a carboxamide via reaction of a primary or secondary amine with a carbonyl halide or anhydride in the presence of an organic or inorganic base is a transformation well documented in the art. Likewise, through the use of chloroformate or isocyanate electrophiles the corresponding carbamate and urea derivatives may be obtained. A representative example of this method to produce a substituted carbonyl derivative of the Formula 7 is shown in Example 2 below.

Method F: It may be desirable to further derivatize the unprotected amine of Formula 5 with substituted alkyl groups to create compounds of interest. A representative example of this method to produce a substituted alkyl derivative of Formula 8 is shown in Example 3 below.

The compounds of the present invention may have asymmetric carbon atoms. Diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of Formula I that are basic in nature are capable of forming a wide variety of different salts with various inorganic and organic acids. Although such salts must be pharmaceutically acceptable for administration to animals, it is often desirable in practice to initially isolate the compound of Formula I from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of Formula I that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of Formula I. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The compounds of the present invention are potent inhibitors of the Aurora family of oncogenic and protooncogenic protein tyrosine kinases such as AUR1 and AUR2 and thus are all adapted to therapeutic use as antiproliferative agents (e.g., anticancer) in mammals, particularly in humans. In particular, the compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hyperplasia of the prostate (e.g., BPH). It is, in addition, expected that a compound of the present invention may possess activity against a range of leukemias and lymphoid malignancies.

The compounds of the present invention may also be useful in the treatment of additional disorders in which aberrant expression ligand/receptor interactions or activation or signalling events related to various protein tyrosine kinases, are involved. Such disorders may include those of neuronal, glial, astrocytal, hypothalamic, and other glandular, macrophagal, epithelial, stromal, and blastocoelic nature in which aberrant function, expression, activation or signalling of the erbB tyrosine kinases are involved. In addition, the compounds of the present invention may have therapeutic utility in inflammatory, angiogenic and immunologic disorders involving both identified and as yet unidentified tyrosine kinases that are inhibited by the compounds of the present invention.

The in vitro activity of the compounds of Formula I may be determined by the following procedure.

This assay measures the activity of recombinant Aurora 2 (AUR2) kinase, specifically the phosphorylation of a peptide substrate, and the potency of inhibitors of Aurora 2 kinase. Product (phosphorylated peptide) is measured by use of a scintillation proximity assay (SPA). The peptide substrate is incubated with gamma 33P-ATP and enzyme and after the designated time the peptide is captured on a streptavidin SPA bead and the extent of phosphorylation is measured by scintillation counting. Inhibition is evaluated based on the ability of inhibitor to reduce phosphorylation relative to the reaction without inhibitor.

The Aurora 2 kinase used in the assay is full length human protein incorporating a His₆ sequence at the N-terminus to facilitate purification. The gene coding this sequence was incorporated into a baculovirus and the virus used to infect SF9 insect cells in culture. The recombinant protein was purified by nickel-agarose affinity chromatography by standard methods.

The reactions are performed in a volume of 50 μL consisting of 25 ng Aurora 2 protein, 50 mM Tris pH8, 10 mM MgCl₂, 1 mM dithiothreitol, 0.1 mM NaVO₄, 0.02% bovine serum albumin, 10 μM ATP, 0.03 μCi ³³P-ATP, and 2 μM biotin-(LRRWSLG)₄ in wells of a 96 well nonbinding surface clear bottom microplate (Wallac Isoplate Cat 1450-514). Compounds are initially dissolved in DMSO, then diluted in 50 mM Tris pH8, 10 mM MgCl₂, 1 mM dithiothreitol, 0.1 mM NaVO₄, 0.02% bovine serum albumin such that 5 μL addition to each well yields the desired final concentration. The reaction is conducted at room temperature for 45 min with gentle shaking, then terminated by addition of 30 μL of Stop Buffer (0.3 mg Streptavidin SPA beads (Amersham), 1:1 water:phosphate buffered saline (0.2 g/L KCl, 0.2 g/L KH₂ PO₄, 8 g/L NaCl, 1.15 g/L Na₂ HPO₄), 0.5% Triton-X, 75 mM EDTA, 375 μM ATP). Cesium chloride (100 μL, 7.5M) is added to each well, the beads are allowed to settle overnight and scintillation counts performed on a Wallac Microbeta Trilux counter. A background correction is made for each based on a zero time reaction. Compound potency is determined as the concentration of inhibitor that produces 50% inhibition relative to the control reaction (without compound), i.e., IC₅₀.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™(tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples, “Ac” means acetyl, “Et” means ethyl, “Me” means methyl, and “Bu” means butyl.

Where HPLC chromatography is referred to in the preparations and examples below, standard conditions well-known to those skilled in the art are employed. For example, the following general conditions may be used wherein a ZORBAX™ RXC18 column (manufactured by Hewlett Packard) of 150 mm distance and 4.6 mm interior diameter is used. The samples are run on a Hewlett Packard-1100 system A gradient solvent method is used running 100 percent ammonium acetate/acetic acid buffer (0.2 M) to 100 percent acetonitrile over 10 minutes. The system then proceeds on a wash cycle with 100 percent acetonitrile for 1.5 minutes and then 100 percent buffer solution for 3 minutes. The flow rate over this period is a constant 3 ml 1 minute.

### Example 1

### Cyclopropanesulfonic acid {5-methyl-3-[4-(5-methyl-1H-pyrazol-3-ylamino)-pyrido[2,3-d]pyrimidin-2-yl]-3-aza-bicyclo[3.1.0]hex-1-yl}-amide

### (A) Preparation of 2-Chloro-N-(3-methyl-1H-pyrazol-5-yl)pyrido[2,3-d]pyrimidine-4-amine

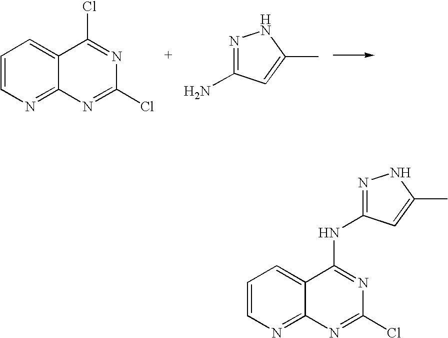

Heat a mixture of 2,4-dichloropyrido[2,3-d]pyrimidine (5.0 g, 1.0 eq.), 3-methyl-1H-pyrazol-5-amine (2.42 g, 1.0 eq.), Diisopropyl ethyl amine (3.54 g, 4.76 mL, 1.1 eq.) and sodium iodide (4.09 g, 1.1 eq.) to 100° C. In Microwave reactor for 1 hour. Concentrate under high vacuum, dilute with EtOAc, filter the solid and wash with water. Dry under high vacuum overnight to obtain 9.75 g the desired compound. 1HNMR (400 MHz, DMSO) δ 9.12 (d, J=8 Hz, 1H), 9.02 (d, J=4 Hz, 1H), 7.61 (q, 1H), 2.29 (s, 3H) HPLC: Rt=4.37 min.

### (B) Preparation of {3-[4-(5-Methyl-1H-pyrazol-3-ylamino)-pyrido[2,3-d]pyrimidin-2-yl]-3-aza-bicyclo[3.1.0]hex-1-yl}-carbamic acid tert-butyl ester

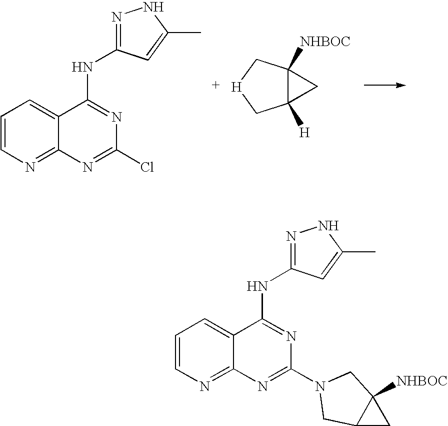

Stir a mixture of 2-Chloro-N-(3-methyl-1H-pyrazol-5-yl)pyrido[2,3-d]pyrimidine-4-amine (500 mg, 1.90 mmol), (3-Aza-bicyclo[3.1.0]hex-1-yl)-carbamic acid tert-butyl ester (418 mg, 2.11 mmol), Diisopropylethyl amine (364 uL, 2.11 mmol) and DMF (8 mL) for 1 h at 90° C. Cool to r.t. and quench with water (80 mL), filter the precipitate. Dilute the filtrate with further 40 mL water and add 20 mL CH₂Cl₂. Add ice and stir for 30 min. Filter the fine white precipitate and combine with the earlier solids. Dissolve all solids in MeOH (reflux) then filter hot. Concentrate the filtrate to dryness to obtain the title compound (240 mg, 30%).

### (C) Preparation of [2-(1-Amino-5-methyl-3-aza-bicyclo[3.1.0]hex-3-yl)-pyrido[2,3-d]pyrimidin-4-yl]-(5-methyl-1H-pyrazol-3-yl)-amine

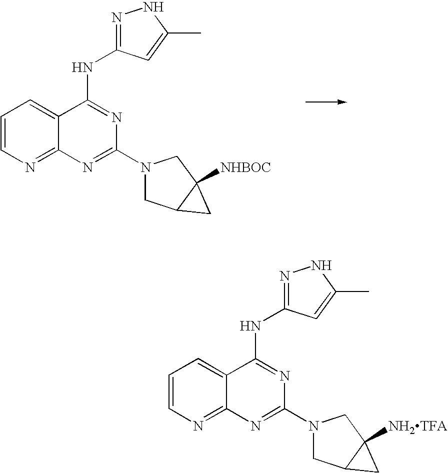

Stir {3-[4-(5-Methyl-1H-pyrazol-3-ylamino)-pyrido[2,3-d]pyrimidin-2-yl]-3-aza-bicyclo[3.1.0]hex-1-yl}-carbamic acid tert-butyl ester (240 mg) in 1:1 TFA/CH₂Cl₂ (6 mL) at RT for 2 h. Remove the solvent in vacuo to afford the title compound as its TFA salt. Triturate the solid with EtOAc/Hexane to afford pure desired product in quantitative yield.

### (D) Preparation of Cyclopropanesulfonic acid {5-methyl-3-[4-(5-methyl-1H-pyrazol-3-ylamino)-pyrido[2,3-d]pyrimidin-2-yl]-3-aza-bicyclo[3.1.0]hex-1-yl}-amide

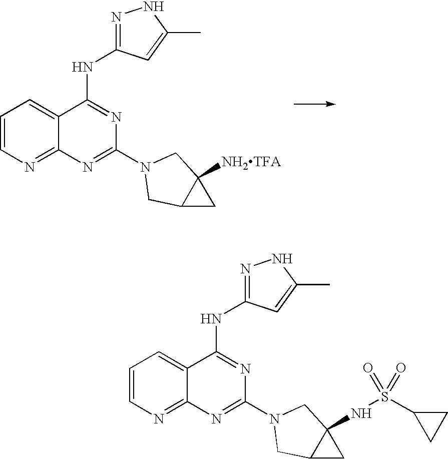

Stir a solution of [2-(1-Amino-5-methyl-3-aza-bicyclo[3.1.0]hex-3-yl)-pyrido[2,3-d]pyrimidin-4-yl]-(5-methyl-1H-pyrazol-3-yl)-amine (65 mg, 0.15 mmol), in 1 mL DMF and excess Et₃N at r.t. for a few minutes. Add cyclopropyl sulfonyl chloride (1.1 eq) stir at r.t. for 1 h. Purify the title compound by flash chromatography by directly loading the reaction mixture to the column. Triturate yellow crystalline material with H₂O to obtain the pure final product. HNMR (400 MHZ, DMSO) δ 8.75 (d, J=7 Hz, 1H), 8.63 (d, J=4 Hz, 1H), 8.00 (s, 1H), 7.06 (m, 1H), 6.6 (br, s, 1H), 4.2 (m, 1H) 3.8 (m, 1H). 3.6 (m, 1H), 2.6 (m, 1H), 2.47 (s, 3H), 1.87 (m, 1H), 1.25 (m, 1H), 1.19 (m, 1H), 1.0 (m, 4H), 0.7 (m, 1H). HPLC Rt: 4.76 min.

### Example 2

### 1-{2-[4-(5-Methyl-2H-pyrazol-3-ylamino)-pyrido[2,3-d]pyrimidin-2-yl]-2,7-diaza-spiro[3,5-d]non-7-yl}-ethanone

### (A) Preparation of 2-[4-(5-Methyl-2H-pyrazol-3-ylamino)-pyrido[2,3-d]pyrimidin-2-yl]-2,7-diaza-spiro[3.5]nonane-7-carboxylic acid tert-butyl ester

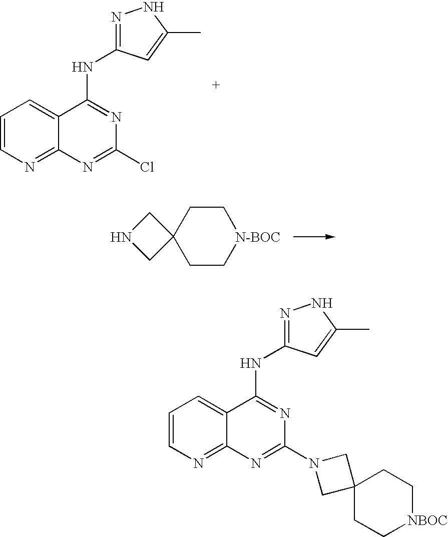

Heat a solution of 2-Chloro-N-(3-methyl-1H-pyrazol-5-yl)pyrido[2,3-d]pyrimidine-4-amine (1.5 g, 5.76 mmol), t-butyl 2,7-diazaspiro[3,5]nonane-7-carboxylate (1.43 g, 6.35 mmol), and N,N-diisopropylethylamine (6.35 mmol, 1.10 mL) in 1-methyl-2-pyrrolidinone (6.0 mL) at 105° C. for 1 h. Cool the reaction to room r.t., dilute with CH₂ Cl₂ and wash with Brine. Dry the organic layer over Na₂SO₄. Concentrate and dilute with EtOAc to provide a solid. Utilize compound without further purification. MS: 451.4 (MH+); HPLC Rt: 6.66 min.; HPLC purity: 86%.

### (B) Preparation of [2-(2,7-Diaza-spiro[3.5]non-2-yl)-pyrido[2,3-d]pyrimidin-4-yl]-(5-methyl-2H-pyrazol-3-yl)-amine

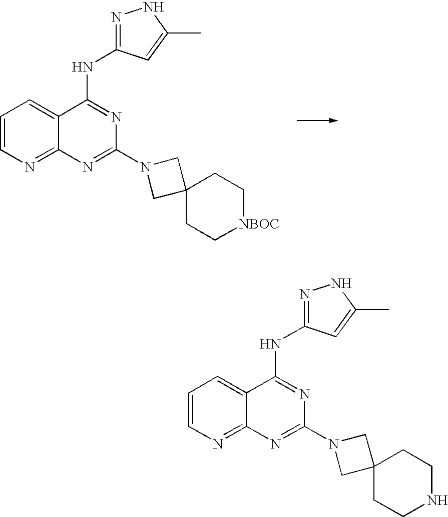

To a solution of 2-[4-(5-Methyl-2H-pyrazol-3-ylamino)-pyrido[2,3-d]pyrimidin-2-yl]-2,7-diaza-spiro[3.5]nonane-7-carboxylic acid tert-butyl ester from the previous step in CH₂ Cl₂ (20 mL) was added TFA (10 mL). Stir the reaction until complete by HPLC analysis. Isolate the title compound (TFA salt) by filtration. MS: 351.4 (MH+); HPLC Rt: 2.79 min.

### (C) 1-{2-[4-(5-Methyl-2H-pyrazol-3-ylamino)-pyrido[2,3-d]pyrimidin-2-yl]-2,7-diaza-spiro[3.5]non-7-yl}-ethanone

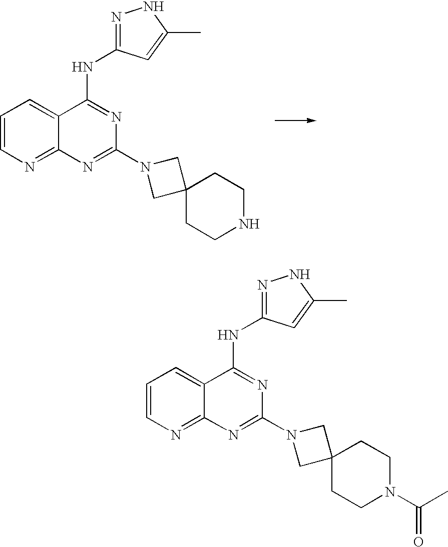

Stir a mixture of [2-(2,7-Diaza-spiro[3.5]non-2-yl)-pyrido[2,3-d]pyrimidin-4-yl]-(5-methyl-2H-pyrazol-3-yl)-amine (120 mg, 0.34 mmol), excess AcCl, excess Et₃ N, and DMF (2 mL) at rt for 1 hr. Concentrate the reaction mixture and treat, with Et₃ N in MeOH at 40° C. for 1 h. Purify the title compound by Prep HPLC (Shimadzu) (18 mg obtained). MS: 393.3 (MH+). HPLC Rt: 3.91, HPLC purity 100%.

### Example 3

### 2-((1S,4S)-5-benzyl-2,5-diaza-bicyclo[2.2.1]heptan-2-yl)-N-(3-cyclopropyl-1H-pyrazol-5-yl)thieno[3,2-d]pyrimidin-4-amine

### (A) Preparation of 2-chloro-N-(3-cyclopropyl-1H-pyrazol-5-yl)thieno[3,2-d]pyrimidin-4-amine

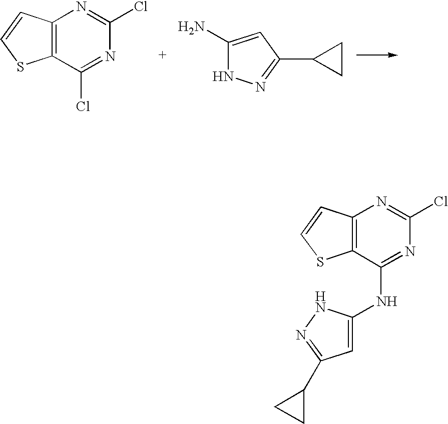

Heat a mixture of 2,4-dichlorothieno[3,2-d]pyrimidine (0.25 mL of 0.4 M NMP solution, 20.5 mg, 0.1 mmol), 3-cyclopropyl-1H-pyrazol-5-amine (0.25 mL of 0.4 M NMP solution, 12.3 mg, 1.0 eq.), Diisopropyl ethyl amine (neat, 74.2 mg, 0.1 mL, 5.7 eq.) to 80° C. in 8-mL vial for 12 hour. After cooling to room temperature, the resulting compound was subjected to next step reaction without further purification.

### (B) Preparation of (1S,4S)-tert-butyl 5-(4-(3-cyclopropyl-1H-pyrazol-5-ylamino)thieno[3,2-d]pyrimidin-2-yl)-2,5-diaza-bicyclo[2.2.1]heptane-2-carboxylate

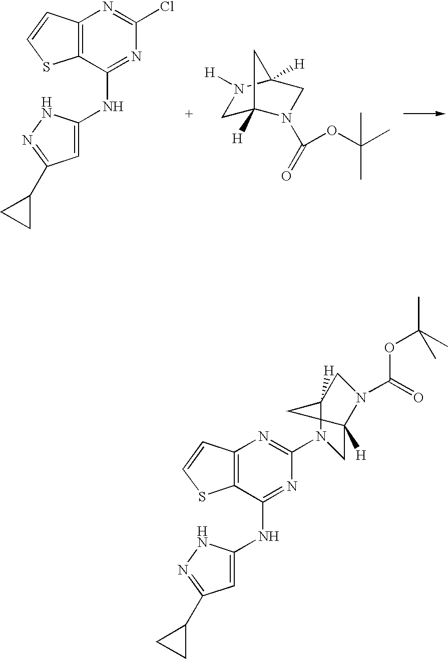

To a solution of 2-chloro-N-(3-cyclopropyl-1H-pyrazol-5-yl)thieno[3,2-d]pyrimidin-4-amine were added (1S,4S)-tert-butyl-2,5-diaza-bicyclo[2.2.1]heptane-2-carboxylate (0.8 mL of 0.25 M NMP solution, 39.65 mg, 2.0 mmol), and Diisopropylethyl amine (neat, 74.2 mg, 0.1 mL, 5.7 eq). The reaction mixture was heated for 3 days at 120° C. Cool to r.t. and remove solvents in Genevac. Add 3 mL of DCE to the vial followed by the addition of 2 mL of 75% ammonium chloride solution. Vortex, centrifuge and transfer 0.27 mL of bottom layer to clean set of vials.

Remove solvents in Genevac, and the resulting compound was subjected to the next step reaction without further purification.

### (C) Preparation of N-(3-cyclopropyl-1H-pyrazol-5-yl)-2-((1S,4S)-5-H-2,5-diaza-bicyclo[2.2.1]heptan-2-yl)thieno[3,2-d]pyrimidin-4-amine

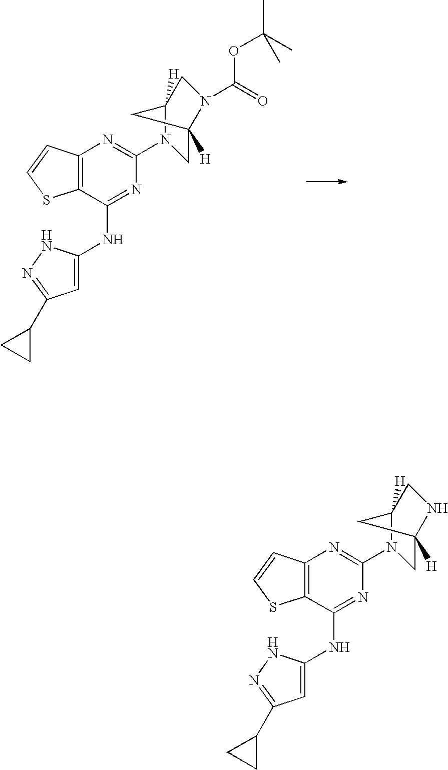

Stir a solution of (1S,4S)-tert-butyl 5-(4-(3-cyclopropyl-1H-pyrazol-5-ylamino)thieno[3,2-d]pyrimidin-2-yl)-2,5-diaza-bicyclo[2.2.1]heptane-2-carboxylate in HCl/methanol (0.6 mL of 4 M HCl—Dioxane solution in 0.8 mL of methanol) at RT for 24 h. Add NH₃/CH₃OH solution (0.6 mL, 4M solution) to the vial, and stir for couple minutes. Remove solvents and the reaction product was subjected to the next step without further purification.

### (D) Preparation of 2-((1S,4S)-5-benzyl-2,5-diaza-bicyclo[2.2.1]heptan-2-yl)-N-(3-cyclopropyl-1H-pyrazol-5-yl)thieno[3,2-d]pyrimidin-4-amine

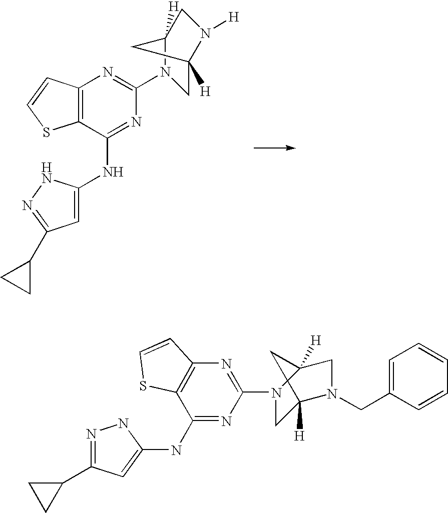

To a solution of N-(3-cyclopropyl-1H-pyrazol-5-yl)-2-((1S,4S)-5-H-2,5-diaza-bicyclo[2.2.1]heptan-2-yl)thieno[3,2-d]pyrimidin-4-amine from previous step was added benzaldehyde (0.6 mL of 0.25 M solution in DCE) and Na[B(OAc)₃]H (1.2 mL of 0.25 M solution in CHCl₃). The reaction mixture was stirred at r.t. for 24 h. Remove solvents, and add 3 mL of DCE to the vial followed by the addition of 2 mL of 75% NH₄Cl solution. Vortex, centrifuge and transfer 2.7 mL of bottom layer to a clean vial. Remove solvent, and purify the title compound by

HPLC. MS: 443.2 (MH+); HPLC Rt: 2.13 min.

### Example 4

### exo-(S)—N2-(7-Aza-bicyclo[2.2.1]hept-2-yl)-N4-(5-methyl-1H-pyrazol-3-yl)-thieno[3,2-d]pyrimidine-2,4-diamine hydrochloride

### (A) Preparation of (2-Chloro-thieno[3,2-d]pyrimidin-4-yl)-(5-methyl-2H-pyrazol-3-yl)-amine

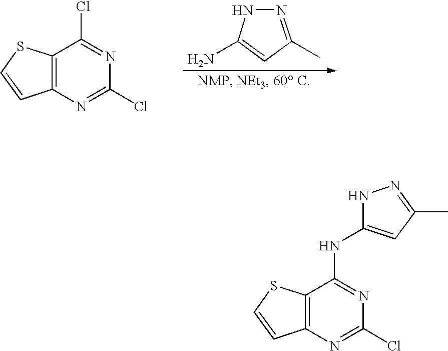

A solution of 2,4-Dichloro-thieno[3,2-d]pyrimidine (1.74 g, 8.48 mmol), 3-methyl-5-aminopyrazole (0.82 g, 8.48 mmol), and triethylamine (16.96 mmol, 2.36 mL) in 1-methyl-2-pyrrolidinone (3 mL) was heated to 60° C. After 2 h the solution was cooled to room temperature and poured into water. The precipitate was collected by filtration and triturated with MeOH to give the title compound as a white solid (1.66 g, 74%). 1HNMR (400 MHz, DMSO) δ 12.3 (br s, 1H), 10.53 (br s, 1H), 8.17 (d, J=13 Hz, 1H), 7.31 (d, J=13 Hz, 1H), 6.32 (br s, 1H), 2.23 (s, 3H); MS: 266.0/267.9 (MH+).

### (B) Preparation of exo-2(S)-[4-(5-Methyl-1H-pyrazol-3-ylamino)-thieno[3,2-d]pyrimidin-2-ylamino]-7-aza-bicyclo[2.21]heptane-7-carboxylic acid tert-butyl ester

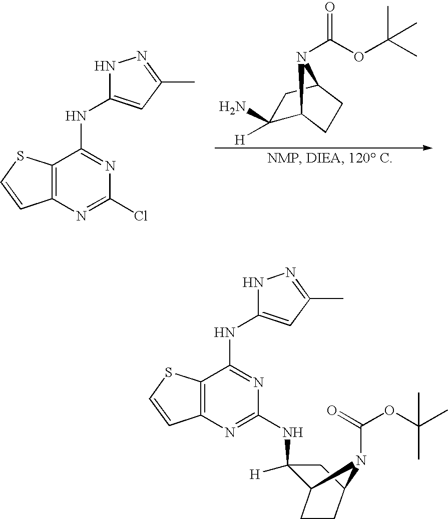

A solution (2-Chloro-thieno[3,2-d]pyrimidin-4-yl)-(5-methyl-2H-pyrazol-3-yl)-amine (250 mg, 0.94 mmol), exo-2(S)-amino-7-aza-bicyclo[2.2.1]heptane-7-carboxylic acid tert-butyl ester (1.0 g, 4.7 mmol), and N,N-diisopropylethylamine (1.88 mmol, 0.34 mL) in 1-methyl-2-pyrrolidinone (0.5 mL) was heated to 120° C. for 24 h. The reaction was cooled to room temperature, diluted with water and extracted with EtOAc. The organic layer was dried over Na2SO4. Purification by Prep HPLC yielded the title compound as a white solid (116 mg, 28%). MS: 442.1/342.1 (MH+); HPLC Rt: 5.5 min.; HPLC purity: 99%.

### (C) Preparation of exo-(S)—N2-(7-Aza-bicyclo[2.2.1]hept-2-yl)-N4-(5-methyl-1H-pyrazol-3-yl)-thieno[3,2-d]pyrimidine-2,4-diamine hydrochloride

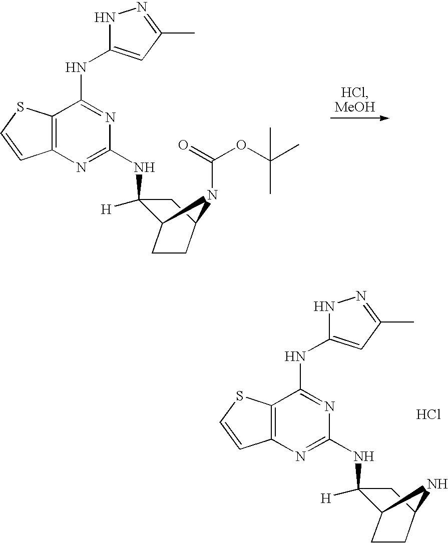

HCl in MeOH (1.25 M, 20 mL) was added to a solution of exo-2(S)-[4-(5-Methyl-1H-pyrazol-3-ylamino)-thieno[3,2-d]pyrimidin-2-ylamino]-7-aza-bicyclo[2.2.1]heptane-7-carboxylic acid tert-butyl ester (88 mg, 0.2 mmol). After 12 h the title compound was isolated by filtration (40 mg, 58%). MS: 342.2 (MH+); HPLC Rt: 3.07 min.; HPLC purity: 100%.

### Example 5

### exo-(R)—N2-7-Aza-bicyclo[2.2.1]hept-2-yl)-N4-(5-methyl-2H-pyrazol-3-yl)-thieno[3,2-d]pyrimidine-2,4-diamine hydrochloride

### (A) Preparation of exo-2(R)-[4-(5-Methyl-2H-pyrazol-3-ylamino)-thieno[3,2-d]pyrimidin-2-ylamino]-7-aza-bicyclo[2.2.1]heptane-7-carboxylic acid tert-butyl ester

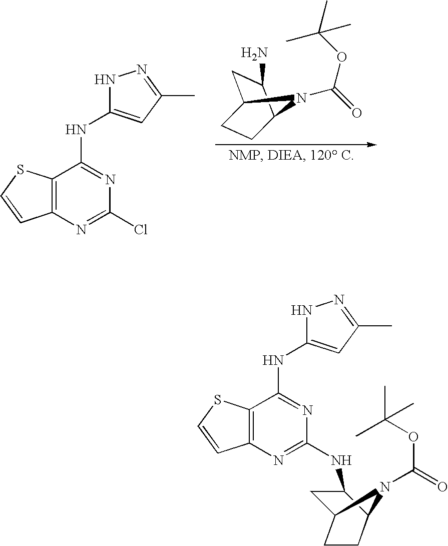

The title compound was prepared from (2-Chloro-thieno[3,2-d]pyrimidin-4-yl)-(5-methyl-2H-pyrazol-3-yl)-amine (0.5 g, 1.88 mmol) and exo-2(R)-amino-7-aza-bicyclo[2.2.1]heptane-7-carboxylic acid tert-butyl ester (2.0 g, 9.4 mmol) by a procedure analogous to that described for exo-2(S)-[4-(5-Methyl-1H-pyrazol-3-ylamino)-thieno[3,2-d]pyrimidin-2-ylamino]-7-aza-bicyclo[2.2.1]heptane-7-carboxylic acid tert-butyl ester. Purification by flash column chromatography (CH₂Cl₂/MeOH 98:2) followed by recrystallization from MeOH afforded the title compound as a white solid (90 mg, 11%). MS: 442.1/342.3 (MH+); HPLC Rt: 5.35 min.; HPLC purity: 100%.

### (B) Preparation of exo-(R)—N2-7-Aza-bicyclo[2.2.1]hept-2-yl)-N4-(5-methyl-2H-pyrazol-3-yl)-thieno[3,2-d]pyrimidine-2,4-diamine hydrochloride

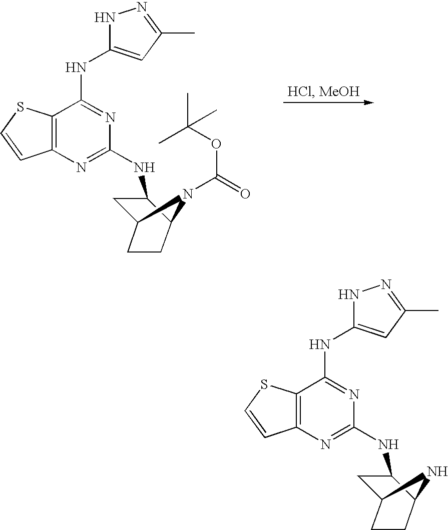

The title compound was prepared from exo-(R)-2-[4-(5-Methyl-2H-pyrazol-3-ylamino)-thieno[3,2-d]pyrimidin-2-ylamino]-7-aza-bicyclo[2.2.1]heptane-7-carboxylic acid tert-butyl ester (90 mg, 0.2 mmol) by a procedure analogous to that described for exo-(S)—N2-(7-Aza-bicyclo[2.2.1]hept-2-yl)-N4-(5-methyl-1H-pyrazol-3-yl)-thieno[3,2-d]pyrimidine-2,4-diamine (50 mg. 65%). MS: 342.3 (MH+); HPLC Rt: 2.95 min.; HPLC purity: 94%.

### Example 6

### exo-benzyl-7-(4-(3-methyl-1H-pyrazol-5-ylamino)thieno[3,2-d]pyrimidin-2-yl)-7-aza-bicyclo[2.2.1]heptan-2(S)-ylcarbamate

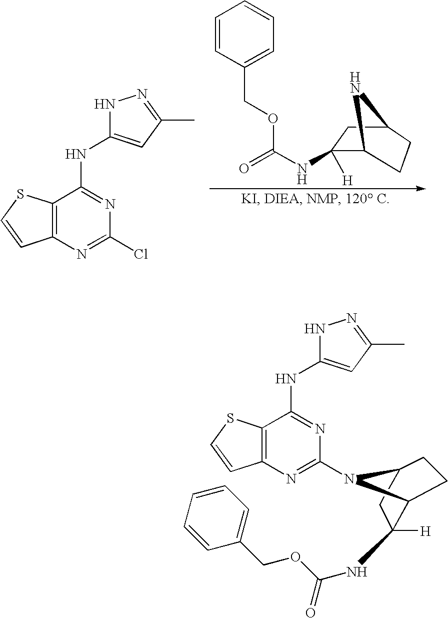

A solution of (2-Chloro-thieno[3,2-d]pyrimidin-4-yl)-(5-methyl-2H-pyrazol-3-yl)-amine (250 mg, 0.94), exo-(7-Aza-bicyclo[2.2.1]hept-2(S)-yl)-carbamic acid benzyl ester (1.1 g, 4.7), KI (156 mg, 0.94), and N,N-diisopropylamine (1.88 mmol, 0.33 mL) in 1-methyl-2-pyrrolidinone (3 mL) was heated to 120° C. for 4 days. The reaction was diluted with water and extracted with EtOAc. The organic layer was dried over Na₂SO₄. Purification by Prep HPLC yielded the title compound as a white solid (45 mg, 10%). MS: 476.3/368.2/342.2 (MH+); LCMS: Rt=1.65 min, 476.4.

### Examples 7-82

The following compounds were prepared via the methods described in Examples 1 to 6 above and the particular method is identified in the table below by the abbreviation “Ex” and example number. In the Table, the term “min” refers to minutes.

| | | | Calculated | | HPLC |
|---|---|---|---|---|---|
| Example | Method | | Molecular | LRMS | Rt |
| Number | of Prep. | IUPAC Name | Weight | (MH+) | (Min) |
| 7 | Ex 2 | {(1S,4S)-5-[4-(5-Cyclopropyl- | 458.548 | 459.09 | 1.16 |
| | | 2H-pyrazol-3-ylamino)- | | | |
| | | thieno[3,2-d]pyrimidin-2-yl]-2,5- | | | |
| | | diaza-bicyclo[2.2.1]hept-2-yl}- | | | |
| | | pyridin-3-yl-methanone | | | |
| 8 | Ex 2 | {(1S,4S)-5-[4-(5-Cyclopropyl- | 459.536 | 460.09 | 1.21 |
| | | 2H-pyrazol-3-ylamino)- | | | |
| | | thieno[3,2-d]pyrimidin-2-yl]-2,5- | | | |
| | | diaza-bicyclo[2.2.1]hept-2-yl}- | | | |
| | | pyrazin-2-yl-methanone | | | |
| 9 | Ex 2 | 1-{(1R,5S)-6-[4-(5-Cyclopropyl- | 425.515 | 426.1 | 1.12 |
| | | 2H-pyrazol-3-ylamino)- | | | |
| | | thieno[3,2-d]pyrimidin-2- | | | |
| | | ylamino]-3-aza- | | | |
| | | bicyclo[3.1.0]hex-3-yl}-2- | | | |
| | | methoxy-ethanone | | | |
| 10 | Ex 2 | 1-{(1R,5S,6S)-6-[4-(5- | 423.543 | 424.12 | 1.39 |
| | | Cyclopropyl-2H-pyrazol-3- | | | |
| | | ylamino)-thieno[3,2-d]pyrimidin- | | | |
| | | 2-ylamino]-3-aza- | | | |
| | | bicyclo[3.1.0]hex-3-yl}-2-methyl- | | | |
| | | propan-1-one | | | |
| 11 | Ex 2 | 1-{(1R,5S)-6-[4-(5-Cyclopropyl- | 471.586 | 472.13 | 1.64 |
| | | 2H-pyrazol-3-ylamino)- | | | |
| | | thieno[3,2-d]pyrimidin-2- | | | |
| | | ylamino]-3-aza- | | | |
| | | bicyclo[3.1.0]hex-3-yl}-2-phenyl- | | | |
| | | ethanone | | | |
| 12 | Ex 1 | (5-Cyclopropyl-2H-pyrazol-3-yl)- | 459.5965 | 460.08 | 2.59 |
| | | {2-[(1S,4S)-5-(propane-2- | | | |
| | | sulfonyl)-2,5-diaza- | | | |
| | | bicyclo[2.2.1]hept-2-yl]- | | | |
| | | thieno[3,2-d]pyrimidin-4-yl}- | | | |
| | | amine | | | |
| 13 | Ex 1 | [2-((1S,4S)-5- | 457.5807 | 458.04 | 2.48 |
| | | Cyclopropanesulfonyl-2,5-diaza- | | | |
| | | bicyclo[2.2.1]hept-2-yl)- | | | |
| | | thieno[3,2-d]pyrimidin-4-yl]-(5- | | | |
| | | cyclopropyl-2H-pyrazol-3-yl)- | | | |
| | | amine | | | |
| 14 | Ex 2 | (1S,4S)-5-[4-(5-Cyclopropyl-2H- | 472.5746 | 473.1 | 2.28 |
| | | pyrazol-3-ylamino)-thieno[3,2- | | | |
| | | d]pyrimidin-2-yl]-2,5-diaza- | | | |
| | | bicyclo[2.2.1]heptane-2- | | | |
| | | carboxylic acid phenylamide | | | |
| 15 | Ex 3 | (1R,5S)-6-[4-(5-Cyclopropyl-2H- | 486.6014 | 487.12 | 2.33 |
| | | pyrazol-3-ylamino)-thieno[3,2- | | | |
| | | d]pyrimidin-2-ylamino]-3-aza- | | | |
| | | bicyclo[3.1.0]hexane-3- | | | |
| | | carboxylic acid benzylamide | | | |
| 16 | Ex 3 | (5-Cyclopropyl-2H-pyrazol-3-yl)- | 395.5325 | 396.1 | 1.8 |
| | | [2-((1S,4S)-5-propyl-2,5-diaza- | | | |
| | | bicyclo[2.2.1]hept-2-yl)- | | | |
| | | thieno[3,2-d]pyrimidin-4-yl]- | | | |
| | | amine | | | |
| 17 | Ex 3 | (5-Cyclopropyl-2H-pyrazol-3-yl)- | 447.5685 | 448.11 | 1.9 |
| | | {2-[(1S,4S)-5-(1-methyl-1H- | | | |
| | | imidazol-2-ylmethyl)-2,5-diaza- | | | |
| | | bicyclo[2.2.1]hept-2-yl]- | | | |
| | | thieno[3,2-d]pyrimidin-4-yl}- | | | |
| | | amine | | | |
| 18 | Ex 3 | (5-Cyclopropyl-2H-pyrazol-3-yl)- | 433.5417 | 434.09 | 1.8 |
| | | {2-[(1S,4S)-5-(1H-imidazol-2- | | | |
| | | ylmethyl)-2,5-diaza- | | | |
| | | bicyclo[2.2.1]hept-2-yl]- | | | |
| | | thieno[3,2-d]pyrimidin-4-yl}- | | | |
| | | amine | | | |
| 19 | Ex 3 | [2-((1S,4S)-5-Benzyl-2,5-diaza- | 443.5765 | 444.09 | 2.13 |
| | | bicyclo[2.2.1]hept-2-yl)- | | | |
| | | thieno[3,2-d]pyrimidin-4-yl]-(5- | | | |
| | | cyclopropyl-2H-pyrazol-3-yl)- | | | |
| | | amine | | | |
| 20 | Ex 3 | (5-Cyclopropyl-2H-pyrazol-3-yl)- | 457.6033 | 458.11 | 2.34 |
| | | [2-((1S,4S)-5-phenethyl-2,5- | | | |
| | | diaza-bicyclo[2.2.1]hept-2-yl)- | | | |
| | | thieno[3,2-d]pyrimidin-4-yl]- | | | |
| | | amine | | | |
| 21 | Ex 3 | (5-Cyclopropyl-2H-pyrazol-3-yl)- | 437.5693 | 438.1 | 1.72 |
| | | {2-[(1S,4S)-5-(tetrahydro-furan- | | | |
| | | 2-ylmethyl)-2,5-diaza- | | | |
| | | bicyclo[2.2.1]hept-2-yl]- | | | |
| | | thieno[3,2-d]pyrimidin-4-yl}- | | | |
| | | amine | | | |
| 22 | Ex 3 | (5-Cyclopropyl-2H-pyrazol-3-yl)- | 434.5258 | 435.05 | 1.78 |
| | | [2-((1S,4S)-5-isoxazol-3- | | | |
| | | ylmethyl-2,5-diaza- | | | |
| | | bicyclo[2.2.1]hept-2-yl)- | | | |
| | | thieno[3,2-d]pyrimidin-4-yl]- | | | |
| | | amine | | | |
| 23 | Ex 3 | {(1S,4S)-5-[4-(5-Cyclopropyl- | 439.5415 | 440.08 | 1.86 |
| | | 2H-pyrazol-3-ylamino)- | | | |
| | | thieno[3,2-d]pyrimidin-2-yl]-2,5- | | | |
| | | diaza-bicyclo[2.2.1]hept-2-yl}- | | | |
| | | acetic acid ethyl ester | | | |
| 24 | Ex 3 | {(1R,5S)-6-[4-(5-Cyclopropyl- | 439.5415 | 440.08 | 1.9 |
| | | 2H-pyrazol-3-ylamino)- | | | |
| | | thieno[3,2-d]pyrimidin-2- | | | |
| | | ylamino]-3-aza- | | | |
| | | bicyclo[3.1.0]hex-3-yl}-acetic | | | |
| | | acid ethyl ester | | | |
| 25 | Ex 1 | N4-(5-Methyl-1H-pyrazol-3-yl)- | 377.4707 | 378.0849 | 1.33 |
| | | N2-[(1R,5S,6S)-3-(propane-2- | | | |
| | | sulfonyl)-3-aza- | | | |
| | | bicyclo[3.1.0]hex-6-yl]- | | | |
| | | pyrimidine-2,4-diamine | | | |
| 26 | Ex 1 | N4-(5-Methyl-1H-pyrazol-3-yl)- | 377.4707 | 378.0782 | 1.19 |
| | | N2-[(1S,5R)-3-(propane-1- | | | |
| | | sulfonyl)-3-aza- | | | |
| | | bicyclo[3.1.0]hex-6-yl]- | | | |
| | | pyrimidine-2,4-diamine | | | |
| 27 | Ex 1 | 3-{(1R,5S)-6-[4-(5-Methyl-1H- | 436.498 | 437.0477 | 1.45 |
| | | pyrazol-3-ylamino)-pyrimidin-2- | | | |
| | | ylamino]-3-aza- | | | |
| | | bicyclo[3.1.0]hexane-3-sulfonyl}- | | | |
| | | benzonitrile | | | |
| 28 | Ex 1 | 3-Cyano-N-{(1R,5S)-3-[4-(5- | 436.498 | 437.0462 | 1.44 |
| | | methyl-1H-pyrazol-3-ylamino)- | | | |
| | | pyrimidin-2-yl]-3-aza- | | | |
| | | bicyclo[3.1.0]hex-6-yl}- | | | |
| | | benzenesulfonamide | | | |
| 29 | Ex 1 | N2-[(1R,5S)-3-(3,5-Dimethyl-1H- | 429.5067 | 430.0651 | 1.27 |
| | | pyrazole-4-sulfonyl)-3-aza- | | | |
| | | bicyclo[3.1.0]hex-6-yl]-N4-(5- | | | |
| | | methyl-1H-pyrazol-3-yl)- | | | |
| | | pyrimidine-2,4-diamine | | | |
| 30 | Ex 2 | (1R,5S)-6-[4-(5-Methyl-1H- | 429.4857 | 430.0995 | 1.26 |
| | | pyrazol-3-ylamino)-pyrimidin-2- | | | |
| | | ylamino]-3-aza- | | | |
| | | bicyclo[3.1.0]hexane-3- | | | |
| | | carboxylic acid (1H-indol-3-yl)- | | | |
| | | amide | | | |
| 31 | Ex 2 | (1R,5S)-6-[4-(5-Methyl-1H- | 418.5024 | 419.1523 | 1.39 |
| | | pyrazol-3-ylamino)-pyrimidin-2- | | | |
| | | ylamino]-3-aza- | | | |
| | | bicyclo[3.1.0]hexane-3- | | | |
| | | carboxylic acid ((R)-1-phenyl- | | | |
| | | ethyl)-amide | | | |
| 32 | Ex 4 | (1R,2S,4S)-2-[4-(5-Methyl-1H- | 441.5573 | 442.1 | 5.5 |
| | | pyrazol-3-ylamino)-thieno[3,2- | | | |
| | | d]pyrimidin-2-ylamino]-7-aza- | | | |
| | | bicyclo[2.2.1]heptane-7- | | | |
| | | carboxylic acid tert-butyl ester | | | |
| 33 | Ex 4 | N2-(1R,2S)-7-Aza- | 341.4411 | 342.2 | 3.07 |
| | | bicyclo[2.2.1]hept-2-yl-N4-(5- | | | |
| | | methyl-1H-pyrazol-3-yl)- | | | |
| | | thieno[3,2-d]pyrimidine-2,4- | | | |
| | | diamine | | | |
| 34 | Ex 5 | (1S,2R,4R)-2-[4-(5-Methyl-2H- | 441.5573 | 442.1 | 5.35 |
| | | pyrazol-3-ylamino)-thieno[3,2- | | | |
| | | d]pyrimidin-2-ylamino]-7-aza- | | | |
| | | bicyclo[2.2.1]heptane-7- | | | |
| | | carboxylic acid tert-butyl ester | | | |
| 35 | Ex 5 | N2-(1S,2R,4R)-7-Aza- | 341.4411 | 342.3 | 2.95 |
| | | bicyclo[2.2.1]hept-2-yl-N4-(5- | | | |
| | | methyl-2H-pyrazol-3-yl)- | | | |
| | | thieno[3,2-d]pyrimidine-2,4- | | | |
| | | diamine | | | |
| 36 | Ex 6 | {(1R,2S,4S)-7-[4-(5-Methyl-2H- | 475.5745 | 476.3 | 1.65 |
| | | pyrazol-3-ylamino)-thieno[3,2- | | | |
| | | d]pyrimidin-2-yl]-7-aza- | | | |
| | | bicyclo[2.2.1]hept-2-yl}-carbamic | | | |
| | | acid benzyl ester | | | |
| 37 | Ex 1 | (2-Azetidin-1-yl-pyrido[2,3- | 281.322 | 282.514 | 0.66 |
| | | d]pyrimidin-4-yl)-(5-methyl-2H- | | | |
| | | pyrazol-3-yl)-amine | | | |
| 38 | Ex 1 | {(1R,5S)-3-[4-(5-Methyl-2H- | 422.49 | 423.404 | 0.98 |
| | | pyrazol-3-ylamino)-pyrido[2,3- | | | |
| | | d]pyrimidin-2-yl]-3-aza- | | | |
| | | bicyclo[3.1.0]hex-6-yl}-carbamic | | | |
| | | acid tert-butyl ester | | | |
| 39 | Ex 1 | [2-(2-Aza-bicyclo[2.2.1]hept-2- | 321.386 | 322.4976 | 0.85 |
| | | yl)-pyrido[2,3-d]pyrimidin-4-yl]- | | | |
| | | (5-methyl-2H-pyrazol-3-yl)- | | | |
| | | amine | | | |
| 40 | Ex 1 | (5-Methyl-2H-pyrazol-3-yl)-[2-(2- | 309.375 | 310.5161 | 0.83 |
| | | propyl-aziridin-1-yl)-pyrido[2,3- | | | |
| | | d]pyrimidin-4-yl]-amine | | | |
| 41 | Ex 1 | 4-{1-[4-(5-Methyl-2H-pyrazol-3- | 465.559 | 466.3793 | 0.84 |
| | | ylamino)-pyrido[2,3-d]pyrimidin- | | | |
| | | 2-yl]-azetidin-3-yl}-piperazine-1- | | | |
| | | carboxylic acid tert-butyl ester | | | |
| 42 | Ex 1 | 2-[4-(5-Methyl-2H-pyrazol-3- | 450.544 | 451.3945 | 1.08 |
| | | ylamino)-pyrido[2,3-d]pyrimidin- | | | |
| | | 2-yl]-2,7-diaza-spiro[3.5]nonane- | | | |
| | | 7-carboxylic acid tert-butyl ester | | | |
| 43 | Ex 1 | 2-Methoxy-N-{(1S,5R)-3-[4-(5- | 394.4368 | 395.4 | 3.57 |
| | | methyl-2H-pyrazol-3-ylamino)- | | | |
| | | pyrido[2,3-d]pyrimidin-2-yl]-3- | | | |
| | | aza-bicyclo[3.1.0]hex-6-yl}- | | | |
| | | acetamide | | | |
| 44 | Ex 2 | 5-{2-[(1S,5R)-6-(2-Methoxy- | 466.4994 | 467.4 | 4.80 |
| | | acetylamino)-3-aza- | | | |
| | | bicyclo[3.1.0]hex-3-yl]- | | | |
| | | pyrido[2,3-d]pyrimidin-4- | | | |
| | | ylamino}-3-methyl-pyrazole-1- | | | |
| | | carboxylic acid ethyl ester | | | |
| 45 | Ex 2 | N-{(1S,5R)-3-[4-(5-Methyl-2H- | 364.411 | 365.2 | 3.60 |
| | | pyrazol-3-ylamino)-pyrido[2,3- | | | |
| | | d]pyrimidin-2-yl]-3-aza- | | | |
| | | bicyclo[3.1.0]hex-6-yl}- | | | |
| | | acetamide | | | |
| 46 | Ex 2 | 5-[2-((1S,5R)-6-Acetylamino-3- | 436.4736 | 437.3 | 4.75 |
| | | aza-bicyclo[3.1.0]hex-3-yl)- | | | |
| | | pyrido[2,3-d]pyrimidin-4- | | | |
| | | ylamino]-3-methyl-pyrazole-1- | | | |
| | | carboxylic acid ethyl ester | | | |
| 47 | Ex 2 | Cyclopropanecarboxylic acid | 390.4488 | 391.3 | 4.54 |
| | | {(1S,5R)-3-[4-(5-methyl-2H- | | | |
| | | pyrazol-3-ylamino)-pyrido[2,3- | | | |
| | | d]pyrimidin-2-yl]-3-aza- | | | |
| | | bicyclo[3.1.0]hex-6-yl}-amide | | | |
| 48 | Ex 1 | 5-[2-((1S,5R)-6- | 472.5276 | 473.1 | 5.31 |
| | | Methanesulfonylamino-3-aza- | | | |
| | | bicyclo[3.1.0]hex-3-yl)- | | | |
| | | pyrido[2,3-d]pyrimidin-4- | | | |
| | | ylamino]-3-methyl-pyrazole-1- | | | |
| | | carboxylic acid ethyl ester | | | |
| 49 | Ex 2 | 1-Isopropyl-3-{(1S,5R)-3-[4-(5- | 407.4795 | 408.2 | 5.52 |
| | | methyl-2H-pyrazol-3-ylamino)- | | | |
| | | pyrido[2,3-d]pyrimidin-2-yl]-3- | | | |
| | | aza-bicyclo[3.1.0]hex-6-yl}-urea | | | |
| 50 | Ex 2 | 5-{2-[(1S,5R)-6-(3-Isopropyl- | 479.5421 | 480.3 | 7.04 |
| | | ureido)-3-aza-bicyclo[3.1.0]hex- | | | |
| | | 3-yl]-pyrido[2,3-d]pyrimidin-4- | | | |
| | | ylamino}-3-methyl-pyrazole-1- | | | |
| | | carboxylic acid ethyl ester | | | |
| 51 | Ex 2 | 5-{2-[(1S,5R)-6-(3-Ethyl- | 481.5823 | 482.2 | 6.11 |
| | | thioureido)-3-aza- | | | |
| | | bicyclo[3.1.0]hex-3-yl]- | | | |
| | | pyrido[2,3-d]pyrimidin-4- | | | |
| | | ylamino}-3-methyl-pyrazole-1- | | | |
| | | carboxylic acid ethyl ester | | | |
| 52 | Ex 1 | [2-(7-Methanesulfonyl-2,7-diaza- | 428.5186 | 429.2 | 4.52 |
| | | spiro[3.5]non-2-yl)-pyrido[2,3- | | | |
| | | d]pyrimidin-4-yl]-(5-methyl-2H- | | | |
| | | pyrazol-3-yl)-amine | | | |
| 53 | Ex 2 | 1-{2-[4-(5-Methyl-2H-pyrazol-3- | 392.4646 | 393.3 | 3.91 |
| | | ylamino)-pyrido[2,3-d]pyrimidin- | | | |
| | | 2-yl]-2,7-diaza-spiro[3.5]non-7- | | | |
| | | yl}-ethanone | | | |
| 54 | Ex 1 | [2-((1R,4R)-5-Methanesulfonyl- | 400.465 | 401.3 | 3.97 |
| | | 2,5-diaza-bicyclo[2.2.1]hept-2- | | | |
| | | yl)-pyrido[2,3-d]pyrimidin-4-yl]- | | | |
| | | (5-methyl-2H-pyrazol-3-yl)- | | | |
| | | amine | | | |
| 55 | Ex 1 | (1R,5S)-3-[4-(5-Methyl-1H- | 417.4747 | 418.4 | 4.42 |
| | | pyrazol-3-ylamino)-pyrido[2,3- | | | |
| | | d]pyrimidin-2-yl]-6-morpholin-4- | | | |
| | | yl-3-aza-bicyclo[3.1.0]hexane-6- | | | |
| | | carbonitrile | | | |
| 56 | Ex 1 | [(1R,4S)-2-(2-Aza- | 321.3861 | 322.3 | 4.91 |
| | | bicyclo[2.2.1]hept-2-yl)- | | | |
| | | pyrido[2,3-d]pyrimidin-4-yl]-(5- | | | |
| | | methyl-1H-pyrazol-3-yl)-amine | | | |
| 57 | Ex 1 | {(1S,5R)-3-[4-(5-Methyl-1H- | 422.4904 | 423.3 | 5.72 |
| | | pyrazol-3-ylamino)-pyrido[2,3- | | | |
| | | d]pyrimidin-2-yl]-3-aza- | | | |
| | | bicyclo[3.1.0]hex-1-yl}-carbamic | | | |
| | | acid tert-butyl ester | | | |
| 58 | Ex 1 | [2-((1S,5R)-1-Amino-3-aza- | 322.3742 | 323.2 | 2.92 |
| | | bicyclo[3.1.0]hex-3-yl)- | | | |
| | | pyrido[2,3-d]pyrimidin-4-yl]-(5- | | | |
| | | methyl-1H-pyrazol-3-yl)-amine | | | |
| 59 | Ex 2 | 1-{(1R,4S)-5-[4-(5-Methyl-2H- | 364.411 | 365.4 | 3.62 |
| | | pyrazol-3-ylamino)-pyrido[2,3- | | | |
| | | d]pyrimidin-2-yl]-2,5-diaza- | | | |
| | | bicyclo[2.2.1]hept-2-yl}- | | | |
| | | ethanone | | | |
| 60 | Ex 1 | 2-Methoxy-1-{(1R,4S)-5-[4-(5- | 394.4368 | 395.4 | 3.49 |
| | | methyl-2H-pyrazol-3-ylamino)- | | | |
| | | pyrido[2,3-d]pyrimidin-2-yl]-2,5- | | | |
| | | diaza-bicyclo[2.2.1]hept-2-yl}- | | | |
| | | ethanone | | | |
| 61 | Ex 1 | 2-Methoxy-N-{1-[4-(5-methyl- | 382.4258 | 383.3 | 2.98 |
| | | 2H-pyrazol-3-ylamino)- | | | |
| | | pyrido[2,3-d]pyrimidin-2-yl]- | | | |
| | | azetidin-3-ylmethyl}-acetamide | | | |
| 62 | Ex 1 | Cyclopropanesulfonic acid {1-[4- | 414.4918 | 415.3 | 3.59 |
| | | (5-methyl-2H-pyrazol-3- | | | |
| | | ylamino)-pyrido[2,3-d]pyrimidin- | | | |
| | | 2-yl]-azetidin-3-ylmethyl}-amide | | | |
| 63 | Ex 1 | Cyclopropanesulfonic acid | 426.5028 | 427.3 | 4.77 |
| | | {(1S,5R)-3-[4-(5-methyl-1H- | | | |
| | | pyrazol-3-ylamino)-pyrido[2,3- | | | |
| | | d]pyrimidin-2-yl]-3-aza- | | | |
| | | bicyclo[3.1.0]hex-1-yl}-amide | | | |
| 64 | Ex 2 | [2-((1R,5S)-6-Benzylamino-3- | 412.4986 | 413.3 | 8.16 |
| | | aza-bicyclo[3.1.0]hex-3-yl)- | | | |
| | | pyrido[2,3-d]pyrimidin-4-yl]-(5- | | | |
| | | methyl-1H-pyrazol-3-yl)-amine | | | |
| 65 | Ex 1 | N-{(1S,5R)-3-[6,7-Dimethoxy-4- | 459.5285 | 460.2 | 4.64 |
| | | (5-methyl-2H-pyrazol-3- | | | |
| | | ylamino)-quinazolin-2-yl]-3-aza- | | | |
| | | bicyclo[3.1.0]hex-6-yl}- | | | |
| | | methanesulfonamide | | | |
| 66 | Ex 2 | 1-{2-[6,7-Dimethoxy-4-(5- | 481.5539 | 482.4 | 4.66 |
| | | methyl-2H-pyrazol-3-ylamino)- | | | |
| | | quinazolin-2-yl]-2,7-diaza- | | | |
| | | spiro[3.5]non-7-yl}-2-methoxy- | | | |
| | | ethanone | | | |
| 67 | Ex 2 | 1-{2-[6,7-Dimethoxy-4-(5- | 451.5281 | 452.2 | 4.65 |
| | | methyl-2H-pyrazol-3-ylamino)- | | | |
| | | quinazolin-2-yl]-2,7-diaza- | | | |
| | | spiro[3.5]non-7-yl}-ethanone | | | |
| 68 | Ex 2 | Cyclopropyl-{2-[6,7-dimethoxy- | 477.5659 | 478.2 | 4.61 |
| | | 4-(5-methyl-2H-pyrazol-3- | | | |
| | | ylamino)-quinazolin-2-yl]-2,7- | | | |
| | | diaza-spiro[3.5]non-7-yl}- | | | |
| | | methanone | | | |
| 69 | Ex 2 | 1-{2-[6,7-Dimethoxy-4-(5- | 479.5817 | 480.2 | 5.40 |
| | | methyl-2H-pyrazol-3-ylamino)- | | | |
| | | quinazolin-2-yl]-2,7-diaza- | | | |
| | | spiro[3.5]non-7-yl}-2-methyl- | | | |
| | | propan-1-one | | | |
| 70 | Ex 1 | [2-(7-Methanesulfonyl-2,7-diaza- | 487.5821 | 488.4 | 5.13 |
| | | spiro[3.5]non-2-yl)-6,7- | | | |
| | | dimethoxy-quinazolin-4-yl]-(5- | | | |
| | | methyl-2H-pyrazol-3-yl)-amine | | | |
| 71 | Ex 2 | 2-[6,7-Dimethoxy-4-(5-methyl- | 467.5271 | 468.2 | 5.37 |
| | | 2H-pyrazol-3-ylamino)- | | | |
| | | quinazolin-2-yl]-2,7-diaza- | | | |
| | | spiro[3.5]nonane-7-carboxylic | | | |
| | | acid methyl ester | | | |
| 72 | Ex 1 | [2-(7-Ethanesulfonyl-2,7-diaza- | 501.6089 | 502.2 | 5.44 |
| | | spiro[3.5]non-2-yl)-6,7- | | | |
| | | dimethoxy-quinazolin-4-yl]-(5- | | | |
| | | methyl-2H-pyrazol-3-yl)-amine | | | |
| 73 | Ex 2 | 2-[6,7-Dimethoxy-4-(5-methyl- | 480.5698 | 481.3 | 4.83 |
| | | 2H-pyrazol-3-ylamino)- | | | |
| | | quinazolin-2-yl]-2,7-diaza- | | | |
| | | spiro[3.5]nonane-7-carboxylic | | | |
| | | acid ethylamide | | | |
| 74 | Ex 2 | N-{1-[6,7-Dimethoxy-4-(5- | 427.4625 | 428.2 | 5.11 |
| | | methyl-2H-pyrazol-3-ylamino)- | | | |
| | | quinazolin-2-yl]-azetidin-3-yl}-2- | | | |
| | | methoxy-acetamide | | | |
| 75 | Ex 1 | N-{1-[6,7-Dimethoxy-4-(5- | 433.4907 | 434.3 | 4.89 |
| | | methyl-2H-pyrazol-3-ylamino)- | | | |
| | | quinazolin-2-yl]-azetidin-3-yl}- | | | |
| | | methanesulfonamide | | | |
| 76 | Ex 1 | Ethanesulfonic acid {1-[6,7- | 447.5175 | 448.1 | 4.66 |
| | | dimethoxy-4-(5-methyl-2H- | | | |
| | | pyrazol-3-ylamino)-quinazolin-2- | | | |
| | | yl]-azetidin-3-yl}-amide | | | |
| 77 | Ex 2 | 2-Methoxy-1-{(1S,4S)-5-[8- | 423.4745 | 424.3 | 3.72 |
| | | methoxy-4-(5-methyl-2H- | | | |
| | | pyrazol-3-ylamino)-quinazolin-2- | | | |
| | | yl]-2,5-diaza-bicyclo[2.2.1]hept- | | | |
| | | 2-yl}-ethanone | | | |
| 78 | Ex 1 | [2-((1S,4S)-5-Methanesulfonyl- | 429.5027 | 430.4 | 4.74 |
| | | 2,5-diaza-bicyclo[2.2.1]hept-2- | | | |
| | | yl)-8-methoxy-quinazolin-4-yl]- | | | |
| | | (5-methyl-2H-pyrazol-3-yl)- | | | |
| | | amine | | | |
| 79 | Ex 2 | (1S,4S)-5-[8-Methoxy-4-(5- | 409.4477 | 410.9 | 4.64 |
| | | methyl-2H-pyrazol-3-ylamino)- | | | |
| | | quinazolin-2-yl]-2,5-diaza- | | | |
| | | bicyclo[2.2.1]heptane-2- | | | |
| | | carboxylic acid methyl ester | | | |
| 80 | Ex 1 | {(1R,5S)-3-[8-Methoxy-4-(5- | 451.5281 | 452.4 | 6.17 |
| | | methyl-1H-pyrazol-3-ylamino)- | | | |
| | | quinazolin-2-yl]-3-aza- | | | |
| | | bicyclo[3.1.0]hex-6-yl}-carbamic | | | |
| | | acid tert-butyl ester | | | |
| 81 | Ex 1 | {(1R,5S)-3-[8-Methoxy-4-(5- | 451.5281 | 452.2 | 6.22 |
| | | methyl-1H-pyrazol-3-ylamino)- | | | |
| | | quinazolin-2-yl]-3-aza- | | | |
| | | bicyclo[3.1.0]hex-1-yl)}-carbamic | | | |
| | | acid tert-butyl ester | | | |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed and such expected variations or differences in the results are contemplated in accordance with the objects and practices of the present invention. It is intended, therefore, that the invention be defined by the scope of the claims which follow and that such claims be interpreted as broadly as is reasonable.

1-12. (canceled)

13. A compound of Formula I: 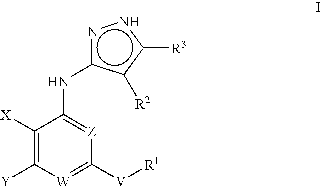
or a pharmaceutically acceptable salt thereof, wherein:
W is N or CR⁴ and Z is N or CH, wherein at least one of W and Z is N;
R¹ is a 3 to 4 membered heterocyclyl ring, said heterocyclyl ring having 1 heteroatom selected from N, O, or S, wherein each substitutable carbon atom in the ring is independently substituted by oxo, -T-R⁴, or -L-Q-R⁴, and each substitutable nitrogen in the ring is independently substituted by R⁵; or R¹ is a 5 to 7 membered bicyclic ring selected from heteroaryl, heterocyclyl, or carbocyclyl, wherein said heteroaryl or heterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵; or R¹ is a 6 to 13 membered spiroheterocyclyl ring, said spiroheterocyclyl ring having 1 to 4 heteroatoms selected from N, O, or S, wherein each substitutable ring carbon in the ring is independently substituted by 1 to 2 substituents selected from oxo, -T-R⁴, or -L-Q-R⁴, and each substitutable ring nitrogen in the ring is independently substituted by R⁵;
V is selected from the group consisting of a bond, —N(R⁵)—, —O—, —S—, —C(R⁵)₂—, and (C₁-C₁₀)alkyl, wherein a methylene unit of said (C₁-C₁₀)alkyl group is optionally replaced by a unit consisting of —O—, —S—, —N(R⁵)—, —CO—, —CONH—, —NHCO—, —SO₂—, —SO₂NH—, —NHSO₂—, —CO₂—, —OC(O)—, —OC(O)NH—, and —NHCO₂;
X and Y are taken together with their intervening atoms to form a fused ring having the structure: 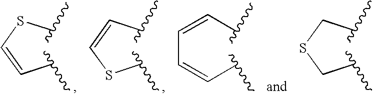
wherein each substitutable ring carbon of said fused ring is independently substituted by 1 to 2 substituents selected from oxo, -T-R⁴, or -L-Q-R⁴;
each T is independently selected from the group consisting of a bond and —(C₁-C₁₀)alkyl, wherein a methylene unit of said (C₁-C₁₀)alkyl group is optionally replaced by a unit consisting of —O—, —S—, —N(R⁵)—, —CO—, —CONH—, —NHCO—, —SO₂—, —SO₂NH—, —NHSO₂—, —CO₂—, —OC(O)—, —OC(O)NH—, and —NHCO₂—;
each Q is independently selected from —(C₁-C₁₀)alkyl;
each L is independently selected from the group consisting of —O—, —S—, —SO₂—, —N(R⁶)SO₂, —SO₂N(R⁶)—, —N(R⁶)—, —CO—, —CO₂—, —C(R⁶)OC(O)—, —C(R⁶)OC(O)N(R⁶)—, —N(R⁶)CO—, —N(R⁶)C(O)O—, —N(R⁶)CON(R⁶)—, —N(R⁶)SO₂N(R⁶)—, —N(R⁶)N(R⁶)—, —C(O)N(R⁶)—, —OC(O)N(R⁶)—, —C(R⁶)₂O—, —C(R⁶)₂S—, —C(R⁶)₂SO—, C(R⁶)₂SO₂—, —C(R⁶)₂SO₂N(R⁶)—, —C(R⁶)N₂(R⁶)—, —C(R⁶)₂N(R⁶)C(O)—, —C(R⁶)₂N(R⁶)C(O)O—, —C(R⁶)═NN(R⁶)—, —C(R⁶)═N—O—, —C(R⁶)₂N(R⁶)N(R⁶)—, —C(R⁶)₂N(R⁶)SO₂N(R⁶)—, and —C(R⁶)₂N(R⁶)CON(R⁶)—;
R² and R³ are independently selected from -T-L-R⁶ and —R⁷; or R² and R³ are taken together with their intervening atoms to form a fused 5 to 9 membered ring having 0 to 3 ring heteroatoms selected from N, O, or S, wherein each substitutable ring carbon of said fused ring is independently substituted by halo, oxo, —CN, —NO₂, —R⁶, and -L-R⁶, and each substitutable ring nitrogen of said ring is independently substituted by R⁵;
R⁴ is selected from the group consisting of —H, halo, —CN, —R⁷, —OR⁷, —C(O)R⁷, —CO₂R⁷, —COCOR⁷, —NO₂, —S(O)R⁷, —SO₂R⁷, —SR⁷, —N(R⁵)₂, —CON(R⁵)₂, —SO₂N(R⁵)₂, —OC(O)R⁷, —N(R⁵)COR⁷, —N(R⁵)CO₂R⁷, —N(R⁵)C═SN(R⁵)₂, —N(R⁵)N(R⁵)₂, —C═NN(R⁵)₂, —C═NOR⁷, —N(R⁵)CON(R⁵)₂, —N(R⁵)SO₂N(R⁵)₂, —N(R⁷)SO₂R⁷, and —OC(O)N(R⁵)₂;
each R⁵ is independently selected from the group consisting of —R⁶, —COR⁶, —CO₂R⁶, —CON(R⁶)₂, and —SO₂R⁶;
each R⁶ is independently selected from H, —(C₁-C₁₀)alkyl, —(C₃-C₈)cycloalkyl, wherein said alkyl or cycloalkyl are independently optionally substituted by 1 to 3 substituents selected from R⁶; or two R⁶ groups on the same nitrogen atom are taken together with the nitrogen atom to form a 5 to 8 membered heterocyclyl or heteroaryl ring, wherein said heterocyclyl and
heteroaryl rings have an additional 1 to 3 ring heteroatoms selected from N, O, or S; or two R⁶ groups on the same carbon atom are taken together with the carbon atom to form a 3 to 6 membered carbocyclic ring;
each R⁷ is independently selected from the group consisting of H, —(C₁-C₁₀)alkyl, —(C₂-C₆)alkenyl, —(C₂-C₆)alkynyl, —(CH₂)ₙ(C₃-C₈)cycloalkyl, —(CH₂)ₙ(C₆-C₁₀)aryl, —(CH₂)ₙ(5 to 10 membered heteroaryl), and —(CH₂)ₙ(5 to 10 membered heterocyclyl), wherein said heteroaryl and heterocyclyl rings having 1 to 3 ring heteroatoms selected from N, O, or S, wherein said alkyl, alkenyl, alkynyl, cycloalkyl, aryl, heteroaryl, and heterocyclyl are independently optionally substituted by 1 to 3 substituents selected from R⁸;
n is an integer from 0 to 6;
each R⁶ is selected from the group consisting of halo, —CN, —OR⁹, —SR⁹, —SO₂R⁹, —N(R⁹)SO₂R⁹, —SO₂N(R⁹)₂, —N(R⁹)₂, —COR⁹, —CO₂R⁹, —C(R⁹)OC(O)R⁹, —C(R⁹)OC(O)N(R⁹)₂, —N(R⁹)COR⁹, —N(R⁹)C(O)OR⁹, —N(R⁹)CON(R⁹)₂, —N(R⁹)SO₂N(R⁹)₂, —N(R⁹)N(R⁹)₂, —C(O)N(R⁹)₂, —OC(O)N(R⁹)₂, —C(R⁹)₂OR⁹, —C(R⁹)₂ SR⁹, —C(R⁹)₂SOR⁹, —C(R⁹)₂SO₂R⁹, —C(R⁹)₂S₂N(R⁹)₂, —C(R⁹)₂N(R⁹)₂, —C(R⁹)₂N(R⁹)C(O)R⁹, —C(R⁹)₂N(R⁹)C(O)OR⁹, —C(R⁹)═NN(R⁹)₂, —C(R⁹)═NOR⁹, —C(R⁹)₂N(R⁹)N(R⁹)₂, —C(R⁹)₂N(R⁹)SO₂N(R⁹)₂, and —C(R⁹)₂N(R⁹)CON(R⁹)₂; and
each R⁹ is independently selected from H, —(C₁-C₁₀)alkyl, —(C₃-C⁸)cycloalkyl or two R⁹ groups on the same nitrogen atom may be taken together with the nitrogen atom to form a 5 to 8 membered heterocyclyl or heteroaryl ring, wherein said heterocyclyl and heteroaryl rings having 1 to 3 ring heteroatoms selected from N, O, or S, or two R⁹ groups on the same carbon atom may be taken together with the carbon atom to form a 3 to 6 membered carbocyclic ring.

14. The compound according to claim 1, wherein W is N and Z is CH.

15. The compound according to claim 1, wherein W is N and Z is N.

16. The compound according to claim 1, wherein W is CR⁴ and Z is N.

17. The compound according to claim 1, wherein V is selected from the group consisting of a bond, —N(R⁵)—, —O—, —C(R⁶)₂—, and (C₁-C₁₀)alkyl, wherein a methylene unit of said (C₁-C₁₀)alkyl group is optionally replaced by a unit consisting of —O—, —S—, —N(R⁵)—, —CO—, —CONH—, —NHCO—, —SO₂—, —SO₂NH—, —NHSO₂—, —CO₂—, —OC(O)—, —OC(O)NH—, and —NHCO₂.

18. The compound according to any of the preceding claims, wherein X and Y are taken together with their intervening atoms to form a fused ring having the structure: 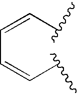
wherein each substitutable ring carbon of said fused ring is independently substituted by 1 to 2 substituents selected from oxo, -T-R⁴, or -L-Q-R⁴.

19. The compound according to claim 1, selected from the group consisting of:
2-((1S,4S)-5-benzyl-2,5-diaza-bicyclo[2.2.1]heptan-2-yl)-N-(3-cyclopropyl-1H-pyrazol-5-yl)thieno[3,2-d]pyrimidin-4-amine;
exo-(S)—N2-(7-Aza-bicyclo[2.2.1]hept-2-yl)-N4-(5-methyl-1H-pyrazol-3-yl)-thieno[3,2-d]pyrimidine-2,4-diamine hydrochloride;
exo-(R)—N2-7-Aza-bicyclo[2.2.1]hept-2-yl)-N4-(5-methyl-2H-pyrazol-3-yl)-thieno[3,2-d]pyrimidine-2,4-diamine hydrochloride;
exo-benzyl-7-(4-(3-methyl-1H-pyrazol-5-ylamino)thieno[3,2-d]pyrimidin-2-yl)-7-aza-bicyclo[2.2.1]heptan-2(S)-ylcarbamate;
{(1S,4S)-5-[4-(5-Cyclopropyl-2H-pyrazol-3-ylamino)-thieno[3,2-d]pyrimidin-2-yl]-2,5-diaza-bicyclo[2.2.1]hept-2-yl}-pyridin-3-yl-methanone;
{(1S,4S)-5-[4-(5-Cyclopropyl-2H-pyrazol-3-ylamino)-thieno[3,2-d]pyrimidin-2-yl]-2,5-diaza-bicyclo[2.2.1]hept-2-yl}-pyrazin-2-yl-methanone;
1-{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-methoxy-ethanone,
1-{(1R,5S,6S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-methyl-propan-1-one,
1-{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-phenyl-ethanone;
(5-Cyclopropyl-2H-pyrazol-3-yl)-{2-[(1S,4S)-5-(propane-2-sulfonyl)-2,5-diaza-bicyclo[2.2.1]hept-2-yl]-thieno[3,2-d]pyrimidin-4-yl}-amine;
[2-((1S,4S)-5-Cyclopropanesulfonyl-2,5-diaza-bicyclo[2.2.1]hept-2-yl)-thieno[3,2-d]pyrimidin-4-yl]-(5-cyclopropyl-2H-pyrazol-3-yl)-amine;
(1S,4S)-5-[4-(5-Cyclopropyl-2H-pyrazol-3-ylamino)-thieno[3,2-d]pyrimdin-2-yl]-2,5-diaza-bicyclo[2.2.1]heptane-2-carboxylic acid phenylamide;
(1R,5S)-6-[4-(5-Cyclopropyl-2H-pyrazol-3-ylamino)-thieno[3,2-d]pyrimidin-2-ylamino]-3-aza-bicyclo[3.1.0]hexane-3-carboxylic acid benzylamide;
(5-Cyclopropyl-2H-pyrazol-3-yl)-[2-((1S,4S)-5-propyl-2,5-diaza-bicyclo[2.2.1]hept-2-yl)-thieno[3,2-d]pyrimidin-4-yl]-amine;
(5-Cyclopropyl-2H-pyrazol-3-yl)-{2-[(1S,4S)-5-(1-methyl-1H-imidazol-2-ylmethyl)-2,5-diaza-bicyclo[2.2.1]hept-2-yl]thieno[3,2-d]pyrimidin-4-yl}-amine;
(5-Cyclopropyl-2H-pyrazol-3-yl)-{2-[(1S,4S)-5-(1H-imidazol-2-ylmethyl)-2,5-diaza-bicyclo[2.2.1]hept-2-yl]-thieno[3,2-c]pyrimidin-4-yl}-amine;
[2-((1S,4S)-5-Benzyl-2,5-diaza-bicyclo[2.2.1]hept-2-yl)-thieno[3,2-d]pyrimidin-4-yl]-(5-cyclopropyl-2H-pyrazol-3-yl)-amine;
(5-Cyclopropyl-2H-pyrazol-3-yl)-[2-((1S,4S)-5-phenethyl-2,5-diaza-bicyclo[2.2.1]hept-2-yl)-thieno[3,2-d]pyrimidin-4-yl]-amine;
(5-Cyclopropyl-2H-pyrazol-1-yl)-{2-[(1S,4S)-5-(tetrahydro-4-furan-2-ylmethyl)-2,5-diaza-bicyclo[2.2.1]hept-2-yl]-thieno[3,2-d]pyrimidin-4-yl}-amine;
(5-Cyclopropyl-2H-pyrazol-3-yl)-[2-((1S,4S)-5-isoxazol-3-ylmethyl-2,5-diaza-bicyclo[2.2.1]hept-2-yl)-thieno[3,2-d]pyrimidin-4-yl]-amine;
{(1S,4S)-5-[4-(5-Cyclopropyl-2H-pyrazol-3-ylamino)-thieno[3,2-d]pyrimidin-2-yl]-2,5-diaza-bicyclo[2.2.1]hept-2-yl}-acetic acid ethyl ester;
{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-acetic acid ethyl ester;
(1R,2S,4S)-2-[4-(5-Methyl-1H-pyrazol-3-ylamino)-thieno[3,2-d]pyrimidin-2-ylamino]-7-aza-bicyclo[2.2.1]heptane-7-carboxylic acid tert-butyl ester;
N2-(1R,2S)-7-Aza-bicyclo[2.2.1]hept-2-yl-N4-(5-methyl-1H-pyrazol-3-yl)-thieno[3,2-d]pyrimidine-2,4-diamine;
(1S,2R,4R)-2-[4-(5-Methyl-2H-pyrazol-3-ylamino)thieno[3,2-d]pyrimidin-2-ylamino]-7-aza-bicyclo[2.2.1]heptane-7-carboxylic acid tert-butyl ester;
N2-(1S,2R,4R)-7-Aza-bicyclo[2.2.1]hept-2-yl-N4-(5-methyl-2H-pyrazol-3-yl)-thieno[3,2-d]pyrimidine-2,4-diamine;
{(1R,2S,4S)-7-[4-(5-Methyl-2H-pyrazol-3-ylamino)-thieno[3,2-d]pyrimidin-2-yl]-7-aza-bicyclo[2.2.1]hept-2-yl}-carbamic acid benzyl ester;
N-{(1S,5R)-3-[6,7-Dimethoxy-4-(5-methyl-2H-pyrazol-3-ylamino)quinazolin-2-yl]-3-aza-bicyclo[3.1.0]hex-6-yl}-methanesulfonamide;
1-{2-[6,7-Dimethoxy-4-(5-methyl-2H-pyrazol-3-ylamino)-quinazolin-2-yl]-2,7-diaza-spiro[3.5]non-7-yl}-2-methoxy-ethanone;
1-{2-[6,7-Dimethoxy-4-(5-methyl-2H-pyrazol-3-ylamino)-quinazolin-2-yl]-2,7-diaza-spiro[3,5]non-7-yl}-ethanone;
Cyclopropyl-{2-[6,7-dimethoxy-4-(5-methyl-2H-pyrazol-3-ylamino)-quinazolin-2-yl]-2,7-diaza-spiro[3.5]non-7-yl}-methanone;
1-{2-[6,7-Dimethoxy-4-(5-methyl-2H-pyrazol-3-ylamino)-quinazolin-2-yl]-2,7-diaza-spiro[3.5]non-7-yl}-2-methyl-propan-1-one;
[2-(7-Methanesulfonyl-2,7-diaza-spiro[3.5]non-2-yl)-6,7-dimethoxy-quinazolin-4-yl]-(5-methyl-2H-pyrazol-3-yl)-amine;
2-[6,7-Dimethoxy-4-(5-methyl-2H-pyrazol-3-ylamino)-quinazolin-2-yl]-2,7-diaza-spiro[3.5]nonane-7-carboxylic acid methyl ester;
[2-(7-Ethanesulfonyl-2,7-diaza-spiro[3.5]non-2-yl)-6,7-dimethoxy-quinazolin-4-yl]-(5-methyl-2H-pyrazol-3-yl)-amine;
2-[6,7-Dimethoxy-4-(5-methyl-2H-pyrazol-3-ylamino)-quinazolin-2-yl]-2,7-diaza-spiro[3.5]nonane-7-carboxylic acid ethylamide;
N-{1-[6,7-Dimethoxy-4-(5-methyl-2H-pyrazol-3-ylamino)-quinazolin-2-yl]-azetidin-3-yl}-2-methoxy-acetamide;
N-{1-[6,7-Dimethoxy-4-(5-methyl-2H-pyrazol-3-ylamino)-quinazolin-2-yl]-azetidin-3-yl}-methanesulfonamide;
Ethanesulfonic acid {1-[6,7-dimethoxy-4-(5-methyl-2H-pyrazol-3-ylamino)-quinazolin-2-yl]-azetidin-3-yl}-amide;
2-Methoxy-1-{(1S,4S)-5-[8-methoxy-4-(5-methyl-2H-pyrazol-3-ylamino)-quinazolin-2-yl]-2,5-diaza-bicyclo[2.2.1]hept-2-yl}-ethanone;
[2-((1S,4S)-5-Methanesulfonyl-2,5-diaza-bicyclo[2.2.1]hept-2-yl)-6-methoxy-quinazolin-4-yl]-(5-methyl-2H-pyrazol-3-yl)-amine;
(1S,4S)-5-[8-Methoxy-4-(5-methyl-2H-pyrazol-3-ylamino)-quinazolin-2-yl]-2,5-diaza-bicyclo[2.2.1]heptane-2-carboxylic acid methyl ester;
{(1R,5S)-3-[8-Methoxy-4-(5-methyl-1H-pyrazol-3-ylamino)-quinazolin-2-yl]-3-aza-bicyclo[3.1.0]hex-6-yl}-carbamic acid tert-butyl ester;
{(1R,6S)-3-[8-Methoxy-4-(5-methyl-1H-pyrazol-3-ylamino)-quinazolin-2-yl]-3-aza-bicyclo[3.1.0]hex-1-yl}-carbamic acid tert-butyl ester;
and the pharmaceutically acceptable salts and solvates of the foregoing compounds.

20. The compound according to claim 1, selected from the group consisting of:
2-((1S,4S)-5-benzyl-2,5-diaza-bicyclo[2.2.1]heptan-2-yl)-N-(3-cyclopropyl-1H-pyrazol-5-yl)thieno[3,2-d]pyrimidin-4-amine;
exo-(S)—N2-(7-Aza-bicyclo[2.2.1]hept-2-yl)-N4-(5-methyl-1H-pyrazol-3-yl)-thieno[3,2-d]pyrimidine-2,4-diamine hydrochloride;
exo-(R)—N2-(7-Aza-bicyclo[2.2.1]hept-2-yl)-N4-(5-methyl-2H-pyrazol-3-yl)-thieno[3,2-d]pyrimidine-2,4-diamine hydrochloride;
exo-benzyl-7-(4-(3-methyl-1H-pyrazol-5-ylamino)thieno[3,2-d]pyrimidin-2-yl)-7-aza-bicyclo[2.2.1]heptan-2(S)-ylcarbamate;
{(1S,4S)-5-[4-(5-cyclopropyl-2H-pyrazol-3-ylamino) thieno[3,2-d]pyrimidin-2-yl]-2,5-diaza-bicyclo[2.2.1]hept-2-yl}-pyridin-3-yl-methanone;
{(1S,4S)-5-[4-(5-Cyclopropyl-2H-pyrazol-3-ylamino)-thieno[3,2-d]pyrimidin-2-yl]-2,5-diaza-bicyclo[2.2.1]hept-2-yl}-pyrazin-2-yl-methanone;
1-{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-methoxy-ethanone;
1-{(1R,5S,6S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-methyl-propan-4-one;
1-{(1R,5S)-6-[4-(5-Cyclopropyl-2H-pyrazol-3-ylamino)-thieno[3,2-d]pyrimidin-2-ylamino]-3-aza-bicyclo[3.1.0]hex-3-yl}-2-phenyl-ethanone;
(5-cyclopropyl-2H-pyrazol-3-yl)-{2-[(1S,4S)-5-(propane-2-sulfonyl)-2,5-diaza-bicyclo[2.2.1]hept-2-yl]-thieno[3,2-d]pyrimidin-4-yl}-amine;
[2-((1S,4S)-5-Cyclopropanesulfonyl-2,5-diaza-bicyclo[2.2.1]hept-2-yl)-thieno[3,2-d]pyrimidin-4 yl]-(5-cyclopropyl-2H-pyrazol-3-yl)-amine;
(1S,4S)-5-[4-(5-Cyclopropyl-2H-pyrazol-3-ylamino)-thieno[3,2-d]pyrimidin-2-yl]-2,5-diaza-bicyclo[2.2.1]heptane-2-carboxylic acid phenylamide;
(1R,5S)-6-[4-(5-Cyclopropyl-2H-pyrazol-3-ylamino)-thieno[3,2-d]pyrimidin-2-ylamino]-3-aza-bicyclo[3.1.0]hexane-3-carboxylic acid benzylamide;
(5-Cyclopropyl-2H-pyrazol-3-yl)-[2-((1S,4S)-5-propyl-2,5-diaza-bicyclo[2.2.1]hept-2-yl)-thieno[3,2-d]pyrimidin-4-yl]-amine;
(5-Cyclopropyl-2H-pyrazol-3-yl)-{2-[(1S,4S)-5-(1-methyl-1H-imidazol-2 ylmethyl)-2,5-diaza-bicyclo[2.2.1]hept-2-yl]-thieno[3,2-d]pyrimidin-4-yl}-amine;
(5-Cyclopropyl-2H-pyrazol-3-yl)-{2-[(1S,4S)-5-(1H-imidazol-2-ylmethyl)-2,5-diaza-bicyclo[2.2.1]hept-2-yl]-thieno[3,2-d]pyrimidin-4-yl}-amine;
[2-((1S,4S)-5-Benzyl-2,5-diaza-bicyclo[2.2.1]hept-2-yl)-thieno[3,2-d]pyrimidin-4-yl]-(5-cyclopropyl-2H-pyrazol-3-yl)-amine;
(5-Cyclopropyl-2H-pyrazol-3-yl)-[2-((1S,4S)-phenethyl-2,5-diaza-bicyclo[2.2.1]hept-2-yl)-thieno[3,2-d]pyrimidin-4-yl]-amine;
(5-Cyclopropyl-2H-pyrazol-3-yl)-{2-[(1S,4S)-5-(tetrahydro-furan-2-ylmethyl)-2,5-diaza-bicyclo[2.2.1]hept-2-yl]-thieno[3,2-d]pyrimidin-4-yl}-amine;
(5-Cyclopropyl-2H-pyrazol-1-yl)-[2-((1S,4S)-5-isoxazol-3-ylmethyl-2,5-diaza-bicyclo[2.2.1]hept-2-yl)thieno[3,2-d]pyrimidin-4-yl]-amine;
{(1S,4S)-5-[4-(5-Methyl-2H-pyrazol-3-ylamino)-thieno[3,2-d]pyrimidin-2-yl]-2,5-diaza-bicyclo[2.2.1]hept-2-yl}-acetic acid ethyl ester;
{(1R,5S)-6-[4-(5-Cyclopropyl-2H-pyrazol-3-ylamino)-thieno[3,2-d]pyrimidin-2-ylamino]-7-aza-bicyclo[3.1.0]hex-3-yl}-acetic acid ethyl ester;
(1R,2S,4S)-2-[4-(5-Methyl-1H-pyrazol-ylamino)-thieno[3,2-d]pyrimidin-2-ylamino]-7-aza-bicyclo[2.2.1]heptane-7-carboxylic acid tert-butyl ester;
N2-(1R,2S)-7-Aza-bicyclo[2.2.1]hept-2-yl-N4-(5-methyl-1H-pyrazol-3-yl)-thieno[3,2-d]pyrimidine-2,4-diamine;
(1S,2R,4R)-2-[4-(5-Methyl-2H-pyrazol-3-ylamino)-thieno[3,2-d]pyrimidin-2-ylamino]-7-aza-bicyclo[2.2.1]heptane-7-carboxylic acid tert-butyl ester;
N2-(1S,2R,4R)-7-Aza-bicyclo[2.2.1]hept-2-yl-N4-(5-methyl-2H-pyrazol-3-yl)-thieno[3,2-d]pyrimidine-2,4-diamine;
{(1S,2S,4S)-7-[4-(5-Methyl-2H-pyrazol-3-ylamino)-thieno[3,2-d]pyrimidin-2-yl]-7-aza-bicyclo[2.2.1]hept-2-yl}-carbamic acid benzyl ester; and the pharmaceutically acceptable salts and solvates of the foregoing compounds.

21. The compound according to claim 1, selected from the group consisting of:
N-{(1S,5R)-3-[6,7-Dimethoxy-4-(5-methyl-2H-pyrazol-3-ylamino)-quinazolin-2-yl]-3-aza-bicyclo[3.1.0]hex-6-yl}-methanesulfonamide;
1-{2-[6,7-Dimethoxy-4-(5-methyl-2H-pyrazol-3-ylamino)-quinazolin-2-yl]-2,7-diaza-spiro[3.5]non-7-yl}-2-methoxy-ethanone;
1-{2-[6,7-Dimethoxy-4-(5-methyl-2H-pyrazol-3-ylamino)-quinazolin-2-yl]-2,7-diaza-spiro[3.5]non-7-yl}-ethanone;
Cyclopropyl-{2-[6,7-dimethoxy-4-(5-methyl-2H-pyrazol-3-ylamino)-quinazolin-2-yl]-2,7-diaza-spiro[3.5]non-7-yl}-methanone;
1-{2-[6,7-Dimethoxy-4-(5-methyl-2H-pyrazol-3-ylamino)-quinazolin-2-yl]-2,7-diaza-spiro[3.5]non-7-yl}-2-methyl-propan-1-one;
[2-(7-Methanesulfonyl-2,7-diaza-spiro[3.5]non-2-yl)-6,7-dimethoxy-quinazolin-4-yl]-(5-methyl-2H-pyrazol-3-yl)-amine;
2-[6,7-Dimethoxy-4-(5-methyl-2H-pyrazol-3-ylamino)-quinazolin-2-yl]-2,7-diaza-spiro[3.5]nonane-7-carboxylic acid methyl ester;
[2-(7-Ethanesulfonyl-2,7-diaza-spiro[3.5]non-2-yl)-6,7-dimethoxy-quinazolin-4-yl]-(5-methyl-2H-pyrazol-3-yl)-amine;
2-[6,7-Dimethoxy-4-(5-methyl-2H-pyrazol-3-ylamino)-quinazolin-2-yl]-2,7-diaza-spiro[3.5]nonane-7-carboxylic acid ethylamide;
N-{1-[6,7-Dimethoxy-4-(5-methyl-2H-pyrazol-3-ylamino)-quinazolin-2-yl]-azetidin-3-yl}-2-methoxy-acetamide;
N-{1-[6,7-Dimethoxy-4-(5-methyl-2H-pyrazol-3-ylamino)-quinazolin-2-yl]-azetidin-3-yl}-methanesulfonamide;
Ethanesulfonic acid {1-[6,7-dimethoxy-4-(5-methyl-2H-pyrazol-3-ylamino)-quinazolin-2-yl]-azetidin-3-yl}-amide;
2-Methoxy-1-{(1S,4S)-5-[8-methoxy-4-(5-methyl-2H-pyrazol-3-ylamino)-quinazolin-2-yl]-2,5-diaza-bicyclo[2.2.1]hept-2-yl}-ethanone;
[2-((1S,4S)-5-Methanesulfonyl-2,5-diaza-bicyclo[2.2.1]hept-2-yl)-8-methoxy-quinazolin-4-yl]-(5-methyl-2H-pyrazol-3-yl)-amine;
(1S,4S)-5-[8-Methoxy-4-(5-methyl-2H-pyrazol-3-ylamino)-quinazolin-2-yl]-2,5-diaza-bicyclo[2.2.1]heptane-2-carboxylic acid methyl ester;
{(1R,5S)-3-[8-Methoxy-4-(5-methyl-1H-pyrazol-3-ylamino)-quinazolin-2-yl]-3-aza-bicyclo[3.1.0]hex-6-yl}-carbamic acid tert-butyl ester;
{(1R,5S)-3-[8-Methoxy-4-(5-methyl-1H-pyrazol-3-ylamino)-quinazolin-2-yl]-3-aza-bicyclo[3.1.0]hex-1-yl}-carbamic acid tert-butyl ester; and the pharmaceutically acceptable salts and solvates of the foregoing compounds.

22. A method for the treatment of abnormal cell growth in a mammal comprising administering to said mammal an amount of a compound of claim 1 that is effective in treating abnormal cell growth.

23. The method according to claim 21, wherein said abnormal cell growth is cancer.

24. A method of preparing a compound of claim 1 which comprises reacting a compound of the Formula II 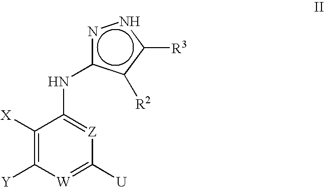
wherein U is a leaving group and W, X, Y, R², and R³ are as defined in claim 1 with a compound of the formula V-R¹, wherein V, and R¹ are as defined in claim 1.
